SEQUENCE LISTING (3 sequences)

SEQ ID NO: 1 - DNA, 27 bp, Artificial
  TaqMan Probe for BVDV
naatcctcctaacaagcgggttccagn

SEQ ID NO: 2 - DNA, 23 bp, Artificial
  Primer (Sense) for BVDV
agccttcagtttcttgctgatgt

SEQ ID NO: 3 - DNA, 20 bp, Artificial
  Primer (Antisense) for BVDV
tgttgcgaaagcaccaacag

(19) United States
(12) Patent Application Publication
(10) Pub. No.: US 2004/0266723 A1
(43) Pub. Date: Dec. 30, 2004

(54) Antiviral agents for treatment of Flaviviridae infections

(76) Inventors: Michael J. Otto, Lilburn, GA (US); Kyoichi A. Watanabe, Stone Mountain, GA (US); Steve Patterson, Dunwoody, GA (US); Krysztof W. Pankiewicz, Atlanta, GA (US); Lieven Stuyver, Snellville, GA (US)

Correspondence Address: Sherry M. Knowles, King & Spalding, 191 Peachtree Street, N.E., 45th Floor, Atlanta, GA, 30303, US

(21) Appl. No.: 10/023,637

(22) Filed: Dec. 17, 2001

Related U.S. Application Data
(60) Provisional application No. 60/256,066, filed on Dec. 15, 2000.

(51) Int. Cl. A61K 31/7076

(52) U.S. Cl. 514/47; 514/48; 536/26.1

(57) ABSTRACT

The disclosed invention is a composition for and a method of treating Flaviviridae (Hepacivirus, Flavivirus, Pestivirus) infections, including BVDV and HCV, in a host, including animals, and especially humans, using a small molecule or its pharmaceutically acceptable salt or prodrug.

This application claims priority to U.S. provisional application 60/256,066 filed on Dec. 15, 2000.

Part of this work was supported by NIH-SBIR grant: 1 R43 CA88578.

### FIELD OF THE INVENTION

The present invention includes compounds and methods for the treatment of Flaviviridae infection such as bovine viral diarrhea virus (“BVDV”), West Nile Virus (WNV) and hepatitis C virus (HCV).

### BACKGROUND OF THE INVENTION

The Flaviviridae is a group of positive single-stranded RNA viruses with a genome size from 9-15 kb. They are enveloped viruses of approximately 40-50 nm. An overview of the Flaviviridae taxonomy is available from the International Committee for Taxonomy of Viruses. The Flaviviridae consists of three genera.

1. Flaviviruses: This genus includes the Dengue virus group (Dengue virus, Dengue virus type 1, Dengue virus type 2, Dengue virus type 3, Dengue virus type 4), the Japanese encephalitis virus group (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), the Modoc virus group, the Rio Bravo virus group (Apoi virus, Rio Brovo virus, Saboya virus), the Ntaya virus group, the Tick-Borne encephalitis group (tick born encephalitis virus), the Tyuleniy virus group, Uganda S virus group and the Yellow Fever virus group. Apart from these major groups, there are some additional Flaviviruses that are unclassified.

2. Hepaciviruses: This genus contains only one species, the Hepatitis C virus (HCV), which is composed of many clades, types and subtypes.

3. Pestiviruses: This genus includes Bovine Viral Diarrhea Virus-2 (BVDV-2), Pestivirus type 1 (including BVDV), pestivirus type 2 (including Hog Cholera Virus) and pestivirus type 3 (including Border Disease Virus).

One of the most important Flaviviridae infections in humans is caused by the hepatitis C virus (HCV). This is the second major cause of viral hepatitis, with an estimated 170 million carriers world-wide (World Health Organization; Hepatitis C: global prevalence, Weekly Epidemiological Record, 1997, 72, 341), 3.9 million of whom reside in the United States (Centers for Disease Control; unpublished data, http://www.cdc.gov/ncidod/diseases/hepatitis/heptab3.htm).

The genomic organization of the Flaviviridae share many common features. The hepatitis C virus (HCV) genome is often used as a model. HCV is a small, enveloped virus with a positive single-stranded RNA genome of ˜9.6 kb within the nucleocapsid. The genome contains a single open reading frame (ORF) encoding a polyprotein of just over 3,000 amino acids, which is cleaved to generate the mature structural and nonstructural viral proteins. The ORF is flanked by 5′ and 3′ non-translated regions (NTRs) of a few hundred nucleotides in length, which are important for RNA translation and replication. The translated polyprotein contains the structural core (C) and envelope proteins (E1, E2, p7) at the N-terminus, followed by the nonstructural proteins (NS2, NS3, NS4A, NS4B, NS5A, NS5B). The mature structural proteins are generated via cleavage by the host signal peptidase (see: Hijikata, M. et al. Proc. Nat. Acad. Sci., USA, 1991, 88, 5547; Hussy, P. et al. Virology, 1996, 224, 93; Lin, C. et al. J. Virol., 1994, 68, 5063; Mizushima, H. et al. J. Virol., 1994, 68, 2731; Mizushima, H. et al. J. Virol., 1994, 68, 6215; Santolini, E. et al J. Virol., 1994, 68, 3631; Selby, M. J. et al. Virology, 1994, 204, 114; and Grakoui, A. et al. Proc. Nat. Acad. Sci., USA, 1993, 90, 10538). The junction between NS2 and NS3 is autocatalytically cleaved by the NS2/NS3 protease (see: Hijikata, M. et al. J. Virol., 1993, 67, 4665 and Bartenschlager, R. et al. J. Virol., 1994, 68, 5045), while the remaining four junctions are cleaved by the N-terminal serine protease domain of NS3 complexed with NS4A (see: Failla, C. et al. J. Virol., 1994, 68, 3753; Lin, C. et al. J. Virol., 1994, 68, 8147; Tanji, Y. et al. J. Virol., 1995, 69, 1575 and Tai, C. L. et al. J. Virol., 1996, 70, 8477). The NS3 protein also contains the Nucleotide Tri-Phosphate-dependent helicase activity which unwinds duplex RNA during replication. The NS5B protein possesses RNA-dependent RNA polymerase (RDRP) activity (see: Behrens, S. E. et al. EMBO J., 1996, 15, 12; Lohmann, V. et al. J. Virol., 1997, 71, 8416-8428 and Lohmann, V. et al. Virology, 1998, 249, 108), which is essential for viral replication (Ferrari, E. et al. J. Virol., 1999, 73, 1649). It is emphasized here that, unlike HBV or HIV, no DNA is involved in the replication of HCV. Recently in vitro experiments using NS5B, substrate specificity for HCV-RDRP was studied using guanosine 5′-monophosphate (GMP), 5′-diphosphate (GDP), 5′-triphosphate (GTP) and the 5′-triphosphate of 2′-deoxy and 2′,3′-dideoxy guanosine (dGTP and ddGTP, respectively). The authors claimed that HCV-RDRP has a strict specificity for ribonucleoside 5′-triphosphates and requires the 2′- and 3′-OH groups (Lohmann; Virology, 108). Their experiments suggest that the presence of 2′- and 3′-substituents would be the prerequisite for nucleoside 5′-triphosphates to interact with HCV-RDRP and to act as substrates or inhibitors.

Examples of antiviral agents that have been identified as active against the hepatitis C flavivirus include:

1. interferon and ribavirin (Battaglia, A. M. et al. Ann. Pharmacother. 2000, 34, 487; Berenguer, M. et al. Antivir. Ther. 1998, 3 (Suppl. 3), 125);

2. Substrate-based NS3 protease inhibitors (Attwood et al. PCT WO 98/22496, 1998; Attwood et al. Antiviral Chemistry and Chemotherapy 1999, 10, 259; Attwood et al. German Patent Pub. DE 19914474; Tung et al. PCT WO 98/17679), including alpha-keto-amides and hydrazinoureas, and inhibitors that terminate in an electrophile such as a boronic acid or phosphonate (Llinas-Brunet et. al. PCT WO 99/07734).

3. Non-substrate-based inhibitors such as 2,4,6-trihydroxy-3-nitro-benzamide derivatives (Sudo K. et al. Biochemical and Biophysical Research Communications, 1997, 238, 643 and Sudo K. et al. Antiviral Chemistry and Chemotherapy 1998, 9, 186), including RD3-4082 and RD3-4078, the former substituted on the amide with a 14 carbon chain and the latter processing a para-phenoxyphenyl group;

4. Thiazolidine derivatives which show relevant inhibition in a reverse-phase HPLC assay with an NS3/4A fusion protein and NS5A/5B substrate (Sudo K. et al. Antiviral Research 1996, 32, 9), especially compound RD-1-6250, possessing a fused cinnamoyl moiety substituted with a long alkyl chain, RD4 6205 and RD4 6193;

5. Thiazolidines and benzanilides identified in Kakiuchi N. et al. J. EBS Letters 421, 217 and Takeshita N. et al. Analytical Biochemistry 1997, 247, 242;

6. A phenanthrenequinone possessing activity against HCV protease in a SDS-PAGE and autoradiography assay isolated from the fermentation culture broth of Streptomyces sp., Sch 68631 (Chu M. et al. Tetrahedron Letters 1996, 37, 7229), and Sch 351633, isolated from the fungus Penicillium griscofuluum, which demonstrates activity in a scintillation proximity assay (Chu, M. et al. Bioorganic and Medicinal Chemistry Letters 9, 1949);

7. Selective NS3 inhibitors based on the macromolecule elgin c, isolated from leech (Qasim, M. A. et al. Biochemistry 1997, 36,1598);

8. HCV helicase inhibitors (Diana, G. D. et al. U.S. Pat. No. 5,633,358 and Diana, G. D. et al. PCT WO 97/36554);

9. HCV polymerase inhibitors such as nucleotide analogues, gliotoxin (Ferrari, R. et al. Journal of Virology 1999, 73, 1649), and the natural product cerulenin (Lohmann, V. et al. Virology 1998, 249, 108);

10. Antisense phosphorothioate oligodeoxynucleotides (S-ODN) complementary to sequence stretches in the 5′ non-coding region (NCR) of the HCV (Alt, M. et al. Hepatology 1995, 22, 707), or nucleotides 326-348 comprising the 3′ end of the NCR and nucleotides 371-388 located in the core coding region of the HCV RNA (Alt, M. et al. Archives of Virology 1997, 142, 589 and Galderisi, U. et al. Journal of Cellular Physiology 1999, 81:2151);

11. Inhibitors of IRES-dependent translation (Ikeda, N et al. Japanese Patent Publication JP-08268890; Kai, Y. et al. Japanese Patent Publication JP-10101591);

12. Nuclease-resistant ribozymes (Maccjak, D. J. et al. Hepatology 1999, 30, abstract 995); and

13. Other miscellaneous compounds including 1-amino-alkylcyclohexanes (Gold et al. U.S. Pat. No. 6,034,134), alkyl lipids (Chojkier et al. U.S. Pat. No. 5,922,757), vitamin E and other antioxidants (Chojkier et al U.S. Pat. No. 5,922,757), squalene, amantadine, bile acids (Ozeki et al. U.S. Pat. No. 5,846,964), N-(phosphonoacetyl)-L-aspartic acid, (Diana et al. U.S. Pat. No. 5,830,905), benzenedicarboxamides (Diana et al. U.S. Pat. No. 5,633,388), polyadenylic acid derivatives (Wang et al. U.S. Pat. No. 5,496,546), 2′,3′-dideoxyinosine (Yarchoan et al. U.S. Pat. No. 5,026,687), and benzimidazoles (Colacino et al. U.S. Pat. No. 5,891,874).

Inosine Monophosphate Dehydrogenase (IMPDH) Inhibitors

The nicotinamide adenine dinucleotide (NAD)-dependent catalysis of the conversion of inosine-5′-monophosphate (IMP) to xanthosine-5′-monophosphate (XMP) by the enzyme IMPDH is the rate limiting step in the de novo pathway of guanosine biosynthesis. Rapidly proliferating cells and/or viral infections are heavily dependent on the availability of large nucleotide pools to meet their metabolic requirements. Compounds blocking this de novo biosynthesis pathway will act selectively on these cell types and leave the other ones substantially unaffected. For example, nucleotide pool production via the salvage pathway alone is sufficient for neural cell and kidney-tissue cell proliferation but not for lymphocytes or cancer cells (Allison, A. C., Eugui, E. M. Transplant. Proc., 1994, 26, 3205). As a consequence of these cell requirements, IMPDH inhibition is a recognized target for immunosuppression, anti-cancer treatment and viral chemotherapy. The biochemical mechanism and structural aspects of enzymatic catalysis and inhibition have been recently reviewed (Hedstrom, L. Curr. Med. Chem., 1999, 6, 545; Goldstein, B. M., Colby, T. D. Curr. Med. Chem., 1999, 6, 519).

There are currently three IMPDH inhibitors on the market. The nucleosides Ribavirin and Mizoribine (Bredinin) are used clinically as antiviral and immunosuppressive drugs, respectively. The non-nucleoside agent mycophenolate mofetil (MMF) is an immunosuppressant used in combination with calcineurin inhibitors such as cyclosporin A or FK-506 in many treatment regimens for the prophylaxis of transplant rejection (Mele, T. S., Halloran, P. F. Immunopharmacology, 2000, 47, 215). These IMPDH inhibitors are typically not used in monotherapy because their efficacious dosing is limited by adverse events, in particular GI or bone marrow toxicity. These toxicities result either from lack of enzyme specificity or unfavorable pharmacokinetics.

The nucleosides require metabolic activation to the corresponding 5′-monophosphates, which competitively bind to the nucleotide site of IMPDH (IMP). Because nucleotide-binding domains are conserved among many enzymes, the action of Ribavirin and Mizoribin is not IMPDH specific. Ribavirin's interaction with guanine monophosphate reductase, guanine phosphoribosyl transferase, deoxycytidine kinase and thymidine kinase has been reported (Prajda, N., Hata, Y., Abonyi, M., Singhal, R. L., Weber, G. Cancer Res., 1993, 53, 5982). Moreover, these two compounds can be further phosphorylated which allows their interference with additional enzymes or their incorporation into DNA. The reported reversible myelotoxicity of Ribavirin in man is typical of cytotoxic drugs (Canonico, P. G., Kastello, M. D., Spears C. T., Brown, J. R., Jackson, E. A., Jenkins, D. E. Tox. Appl. Pharm., 1984, 74, 155). No toxicology data based on clinical practice with Mizoribin is publicly available (Ishikawa, H. Curr. Med. Chem., 1999, 6, 575). See also Pankiewicz, K. P. Exp. Opin. Ther. Patents, 1999, 9, 55.

The non-nucleoside drug mycophenolate mofetil (MMF) is a prodrug of the fungal agent mycophenolic acid (MPA); a highly potent, selective, reversible, noncompetitive IMPDH inhibitor binding at the enzyme's NAD binding site. The favorable activity profile of MMF does not translate into a compound with high clinical efficacy or large therapeutic index. For example, the suppressive effect of MMF on cancer cell lines could not be confirmed in vivo (Tressler, R. J., Garvin, L. J., Slate, D. L. Int. J. Cancer, 1994, 57, 568). The relative efficacy of MPA administered on a compassionate-use basis (mean oral dose: 3.7 g/d for the first year followed by 3.0 g/d for >10 years) to psoriasis patients refractory to conventional therapy was limited by a high incidence of GI toxicity (in 72% of patients during first year) (Epinette, W. W., Parker, C. M., Jones, E. L., Greist, M. C. J. Am. Acad. Derm., 1987, 17, 962). Significant clinical improvement has been seen in many MMF-treated rheumatoid arthritis (RA) patients refractory to other disease-modifying anti-rheumatic drugs. Administration of 2 g/d orally b.i.d reduced rheumatoid factor titters in patient peripheral blood (IgG, IgM, IgA titters T-cell number). However, in spite of the low dose used in comparison to the one employed in the psoriasis trial, GI adverse-effects were again of concern (Grundmann-Kollmann, M., Mooser, G., Schraeder, P., Zollner, T., Kaskel, P., Ochsendorf, F., Boehncke, W. H., Kerscher, M., Kaufmann, R., Peter, R. J. Am. Acad. Derm., 2000, 42, 835; Goldblum, R. Clin. Exp. Rheumatol., 1993, 11 (Suppl 8), S117). The only indication for which MMF has been approved (2- and 3 g/d given orally b.i.d) is for the prevention of solid transplant organ rejection (Mele, T. S., Halloran, P. F. Immunopharmacology, 2000, 47, 215; European Mycophenolate Mofetil Co-operative Study Group. Transplant. Proc., 1997, 29, 2932). In addition to the presence of GI adverse effects, a new finding of leukopenia was also observed (Holt, C. D., Sievers, T. M., Ghobrial, R. M., Rossi, S. J., Goss, J. A., McDiarmid, S. V. BioDrugs, 1998, 10, 373). In spite of its narrow therapeutic index, MMF is widely being used in immunosuppressive regimens for organ transplantation because of the documented substantial reduction of the incidence and intensity of acute organ loss in this life-saving indication.

Numerous pharmacokinetic (PK) investigations have established that MPA, the bioactive component resulting after the rapid and complete enzymatic hydrolysis of orally administered MMF, undergoes extensive enterohepatic recirculation (EHC) (Bullingham, R. E. S., Nicholls, A., Hale, M. Transplant. Proc., 1996, 28, 925). Approximately 80-90% of MPA is efficiently conjugated into the biologically inactive glucuronide (MPAG) mainly in the liver. MPAG is then excreted into the bile, de-glucuronidated by colonic bacteria and absorbed in the gastrointestinal track (GIT), entering the systemic circulation via the portal flow as MPA. As a result of this pronounced EHC, huge concentrations of MPA are present in the GIT resulting in local damage to the intestinal epithelium. This process is manifested as symptoms including nausea, vomiting, loose bowel movements, abdominal pain and diarrhea. As demonstrated in renal transplanted patients, the PK parameter most closely linked to the pharmacodynamic effect and therapeutic efficacy of MMF is the systemic MPA exposure (area under the time-concentration curve, AUC). Consequently, high doses of MMF are required to maintain systemic therapeutic levels (Hale, M. D., Nicholls, A. J., Bullingham, R. E. S., Hene, R., Hoitsma, A., Squifflet, J-P., Weimar, W., Vanrenterghem, Y., Van de Woude, F. J., Verspooten, G. A. Clin. Pharmacol. Ther., 1998, 64, 672; Shaw, L. M., Sollinger, H. W., Halloran, P., Morris, R. E., Yatscoff, R. W., Ransom, J., Tsina, I., Keown, P., Holt, D. W., Lieberman, R., Jaklitsch, A., Potter, J. Ther. Drug Monit., 1995, 17, 690). Any process that would tend to decrease the systemic concentration of MPA will also decrease the therapeutic efficacy of MPA. Therefore, it is reasonable to assume that the reported lack of anti-cancer activity of MPA in vivo can be attributed to its rapid removal from the circulation via drastic glucuronidation by cancer cells (Franklin, T. J., Jacobs, V., Jones, G., Ple, P., Bruneau, P. Cancer Res., 1996, 56, 984).

The replacement or protection of the phenolic group to avoid glucuronidation, the modification of the phthalide ring or the replacement of the lactone oxygen was found to be detrimental for potent IMPDH inhibition. Among the numerous derivatives synthesized, the substitution of the methoxy group by an ethyl chain provided a two-fold higher in vitro and 3.5-fold higher in vivo potency in the murine plaque-forming assay than MPA (Nelson, P. H., Carr, S. F., Devens, B. H., Eugui, E. E., Franco, F., Gonzalez, C., Hawley, R. C., Loughhead, D. G., Milan, D. J., Papp, E., Patterson, J. W., Rouhafza, S., Sjogren, E. B., Smith, D. B., Stephenson, R. A., Talamas, F. X., Waltos, A-M., Weikert, R. J., Wu, J. C. J. Med. Chem.,1996, 39, 4181).

The enzymatic activity was also very sensitive to alterations of MPA's hexenoic chain. While the large majority of these alterations led to compounds of limited biological utility, some interesting MPA derivatives were produced (Nelson, P. H., Eugui, E., Wang, C. C., Allison, A. C. J. Med. Chem., 1990, 33, 833). For example, the conformationally restricted cyclopentenyl analogue inhibited IMPDH with an IC₅₀ of 8 nM versus 20 nM for MPA due to the entropic energy gained by locking MPA into its bioactive conformation (Artis, D. R. et al. WO 95/22538). However, the presence of the phenolic function responsible for enterohepatic recirculation, suggests that, in spite of their minor superior potency, no considerable improvement in the therapeutic indexes of these two inhibitors can be expected. Additionally, MPA derivatives carrying at the hexenoic side chain either a-substituents (benzyl, thiomethyl, methoxymethyl, p-hydroxyphenyl, trifluoroacetamido-phenyl) or a methyl at the e-position were shown to be less susceptible to glucuronidation as assessed using the HT29 cell line which rapidly transforms MPA to MPAG. However, their in vitro efficacy was greatly reduced with the exception of the racemic methoxymethyl derivative, which manifest 29% higher in vitro activity than MPA (Franklin, T. J., Jacobs, V. N., Jones, G., Ple, P. Drug Metab. Disp., 1997, 25, 367). This racemic compound is twice as resistant to glucuronidation as MPA.

In contrast to the structure-activity relationships that can be rationally interpreted based on the architecture of the complex between MPA and IMPDH, the reasons for the resistance of some phenolic compounds to glucuronyltransferases are unclear (Sintchak, M. D., Fleming, M. A., Futer, O., Raybuck, S. A., Chambers, S. P., Caron, P. R., Murcko, M. A., Wilson, K. P. Cell, 1996, 85, 921, Colby, T. D., Vanderveen, K., Strickler, M. D., Markham, G. D., Goldstein, B. M. Proc. Nat. Acad. U.S.A., 1999, 96, 3531). It has been suggested that the additional substituents lead to unfavourable steric interactions with the enzyme's active site (Franklin, T. J., Jacobs, V. N., Jones, G., Ple, P. Drug Metab. Disp., 1997, 25, 367).

In addition to the extensive work aimed at improving the profile of MPA, a recent approach to increase its therapeutic index by influencing the severity of the adverse effects through an alternative galenic formulation has been reported. The sodium salt of MPA (MPS), in an enteric coated delivery form, coded as ERL080, is currently in phase III clinical studies for the prevention of acute renal allograft rejection (Haeberlin, B. et al. WO 9738689). This novel formulation is expected to release the drug in or near the small intestine and thus alleviate the adverse effects of MPA related to high local concentrations in the upper GIT such as anorexia, abdominal pain, nausea or vomiting. The functioning enteric coating of ERL080 was apparent based on the delayed MPA Tₘₐₓ measured in PK studies carried out in renal transplanted patients (2.0 versus 0.8 hours for MMF) (Schmouder, R., Arns, W., Merkel, F., Schoudrhury, S., Russel, D., Taccard, G. Transplantation, 1999, 67(Suppl.), S203). Indeed, the ERL gastro-resistant tablets were rapidly absorbed upon oral administration, leading to systemic MPA exposure bioequivalent to that of MMF capsules. This study also clearly showed that a MPA prodrug form such as MMF is not necessary for the efficient systemic delivery of MPA via the oral route.

Another formulation claimed to improve the therapeutic range of anti-proliferative drugs undergoing EHC, consists of the combination of MMF or MPA with cholestyramine (Lindner, J., et al. WO 0033876). Cholestyramine is a non-absorbable, cationic resin that unspecifically binds bile-acids and any large-sized acidic drug such as MPA and thus blocks the recycling of the parent compound via the EHC route. When cholestyramine was administered to healthy subjects receiving single doses of MMF, exposure to MPA was significantly decreased (mean reduction 37%), this result being consistent with a strong EHC process (Bullingham, R. E. S., Nicholls, A., Hale, M. Transplant. Proc., 1996, 28, 925). Taking into account the established correlation between MPA's pharmacological effects and systemic AUC, a formulation allowing the co-delivery of cholestyramine and MMF would be expected to drastically lower MPA AUC and, consequently, the efficacy of MPA (Hale, M. D., Nicholls, A. J., Bullingham, R. E. S., Hene, R., Hoitsma, A., Squifflet, J-P., Weimar, W., Vanrenterghem, Y., Van de Woude, F. J., Verspooten, G. A. Clin. Pharmacol. Ther., 1998, 64, 672; Shaw, L. M., Sollinger, H. W., Halloran, P., Morris, R. E., Yatscoff, R. W., Ransom, J., Tsina, I., Keown, P., Holt, D. W., Lieberman, R., Jaklitsch, A., Potter, J. Ther. Drug Monit., 1995, 17, 690). No pharmacological data resulting from the above combination treatment has been reported.

U.S. Pat. No. 4,686,234 describes various derivatives of mycophenolic acid, their synthesis and uses in the treatment of autoimmune disorders, psoriasis, and inflammatory diseases, including, in particular, rheumatoid arthritis, tumors, viruses, and for the treatment of allograft rejection.

On May 5, 1995, Morris et al., in U.S. Pat. No. 5,665,728, disclosed a method of preventing or treating hyperproliferative vascular disease in a mammal by administering an antiproliferative effective amount of rapamycin alone or in combination with mycophenolic acid.

It is an objective of the present invention to provide compounds, compositions and methods for the treatment or prophylaxis of immunological disorders, proliferative diseases and viral infections in a host.

In addition, in view of the severity of the illnesses associated with Flaviviridae infections, and their pervasiveness in animal and man, it is another object of the present invention to provide compounds, compositions and methods for the treatment or prophylaxis of Flaviviridae infections in a host.

### SUMMARY OF INVENTION

The present invention provides compounds, compositions and methods for the treatment or prophylaxis of an immunological disorder, abnormal cellular proliferation or viral infection, and in particular a Flaviviridae infection, including an HCV or a BVDV infection, in a host comprising administering an effective agent of the formula (I):

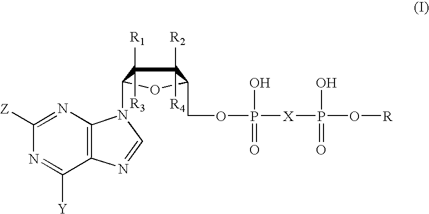

or its pharmaceutically acceptable salt or prodrug thereof; wherein

R is

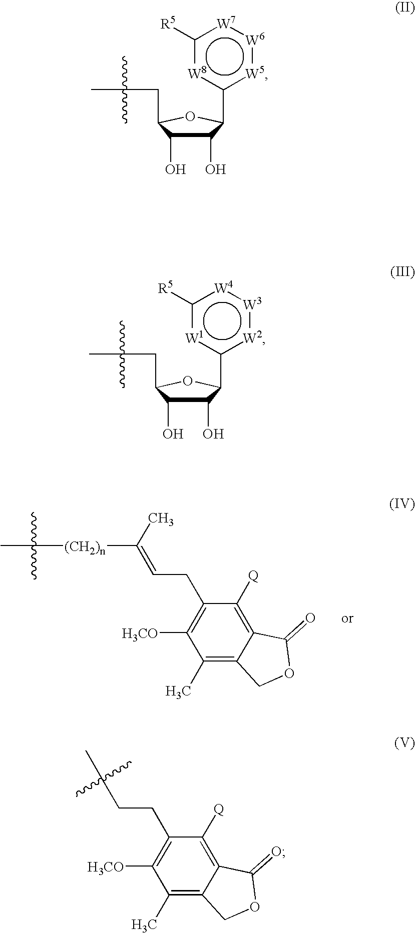

X is oxygen, sulfur, methylene, monofluoromethylene or difluoromethylene;

Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;

Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;

W¹-W⁴ are same or different, and independently methyne (—CH═), azomethyne (—N═) or sulfur;

W⁵-W⁸ are same or different, and independently methyne (—CH═) or azomethyne (—N═);

R¹, R², R³ and R⁴ are independently hydrogen, hydroxyl or fluorine;

R⁵ is halogen (F, Cl, Br, I), CN, CONH₂, CO₂Me, CO₂Et or CO₂H; and

R⁶, R⁷, R⁸ and R⁹ are independently a lower alkane or alkene of 1, 2, 3, 4, 5 or 6 carbons or aryl or aralkyl such as unsubstituted or substituted phenyl or benzyl.

In one embodiment of the present invention, the compound of the general formula (I) is specifically not tiazole-4-carboxamide adenine dinucleotide (TAD) or benzamide adenine dinucleotide (BAD).

In addition, truncated compounds chemically modified as discussed below in order to improve their activity, and in particular antiviral activity, and/or decrease their toxicity are also provided. In particular, the present invention also provides compounds wherein the molecular structures are composed of parts (fragments) of compounds of formula (I), e.g. C2-, C4-, and C6-mycophenolic alcohols, as well as mycophenolic alcohols modified by simple oxidation to the corresponding aldehyde or carboxylic acid derivatives, for example, the following:

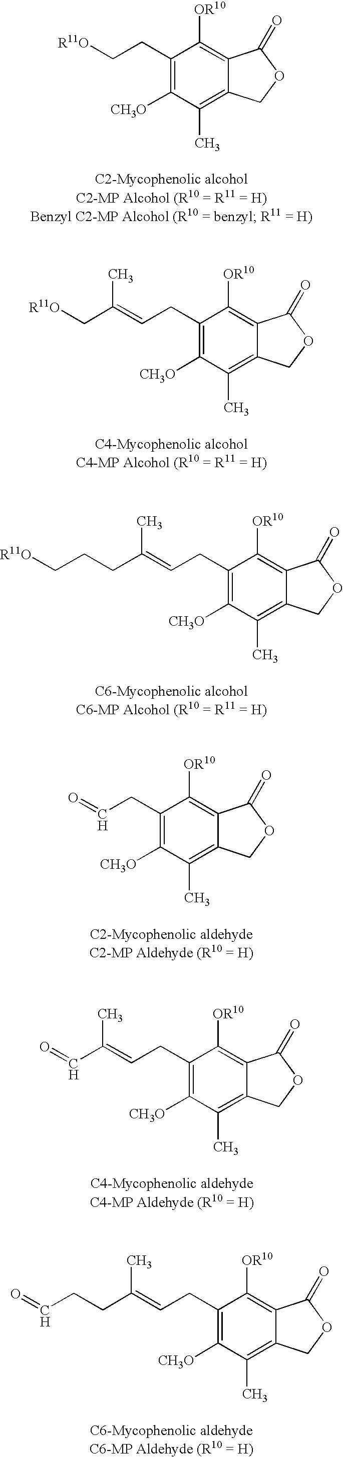

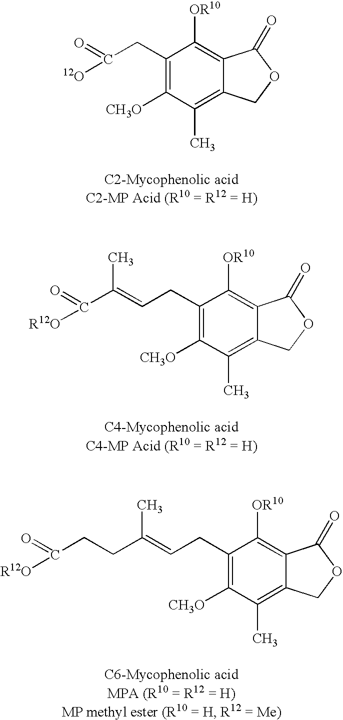

or its pharmaceutically acceptable salt or prodrug, wherein each R¹⁰ and R¹¹ is independently hydrogen, alkyl, acyl, benzyl or methoxymethyl (MOM) group, and each R¹² is independently hydrogen, alkyl or aryl.

These compounds can be modified by replacement of the alcohol, aldehyde or carboxyl group with the corresponding sulfonyl or phosphoryl functional group.

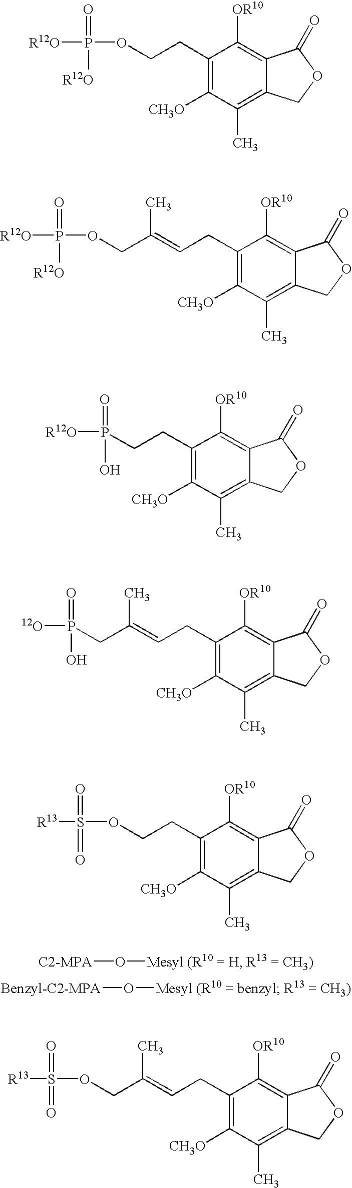

or its pharmaceutically acceptable salt or prodrug thereof, wherein R¹⁰ and R¹² are as defined above, and R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl.

The parent mycophenolic alcohols can also be reduced to the corresponding alkyl derivatives or dehydrated to the alkenyl or alkynyl derivatives. Non-limiting examples include the following:

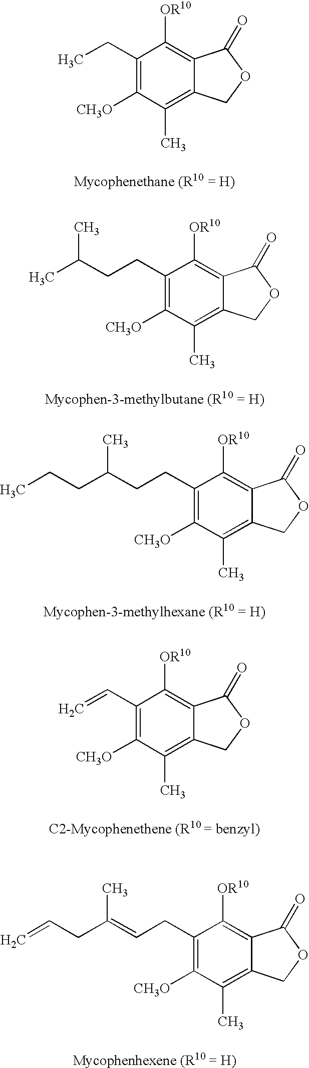

or its pharmaceutically acceptable salt or prodrug thereof, wherein R¹⁰ is as defined above.

Truncated compounds can be composed of larger fragments such as bis(phosphonate) analogues of mycophenolic alcohols. These compounds may be modified by coupling with another mycophenolic alcohol derivative to give a dimer, e.g. bis-C2MPAlcbis(phosphonate), such as the following:

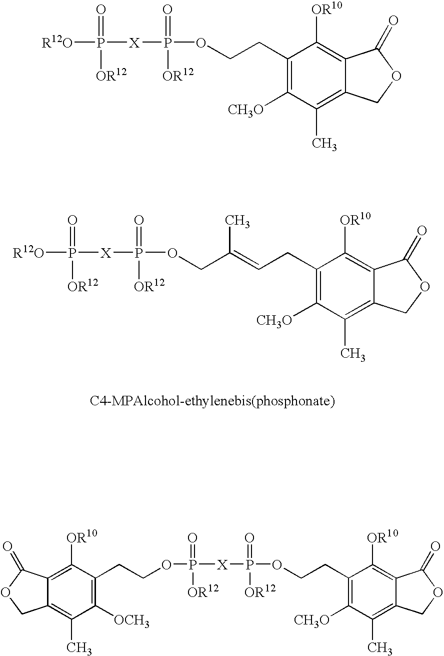

or its pharmaceutically acceptable salt or prodrug thereof, wherein X, R¹⁰ and R¹² are as defined above.

The truncated compounds can also be nucleosides such as tiazofurin, benzamide riboside, C-nicotinamide riboside or F-ara-purines (such as F-ara-A).

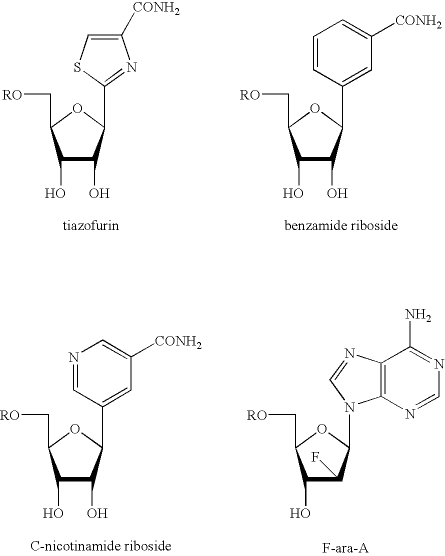

or its pharmaceutically acceptable salt or prodrug, wherein R can be hydrogen, acyl or silyl.

In a preferred embodiment of the invention, the compounds can be administered in the form of an ether lipid. Alternatively, the phosphonates and phosphoryls can be administered in the form of stabilized phosphate or phospholipid.

The present invention includes at least the following features:

a) a compound for the treatment or prophylaxis of a Flaviviridae infection;

b) a process for the preparation of the effective agents described herein, their pharmaceutically acceptable salts and prodrugs thereof;

c) a pharmaceutical composition that includes an effective amount of an agent as described herein, or its pharmaceutically acceptable salt or prodrug thereof together with a pharmaceutically acceptable carrier or diluent according to the present invention for the treatment or prophylaxis of abnormal cellular proliferation and/or viral infection, and in particular a Flaviviridae infection, such as an HCV or BVDV infection, in a host, and in particular in a human;

d) a pharmaceutical composition that includes an effective amount of an agent as described herein, or its pharmaceutically acceptable salt or prodrug thereof in combination with one or more other antivirally effective agents, optionally in a pharmaceutically acceptable carrier or diluent according to the present invention for the treatment or prophylaxis of abnormal cellular proliferation and/or viral infection, and in particular a Flaviviridae infection, such as an HCV or BVDV infection, in a host, and in particular in a human;

e) a method for the treatment or prophylaxis of abnormal cellular proliferation and/or viral infection, and in particular a Flaviviridae infection, including hepatitis C virus or BVDV infection, in a host, and in particular a human, comprising administering an effective amount of an agent as described herein, or its pharmaceutically acceptable salt or prodrug thereof, optionally in a pharmaceutically acceptable carrier or diluent;

f) a method for the treatment or prophylaxis of abnormal cellular proliferation and/or viral infection, and in particular a Flaviviridae infection, including hepatitis C virus or BVDV infection, in a host, an in particular a human, comprising administering an effective amount of an agent as described herein, or its pharmaceutically acceptable salt or prodrug thereof in combination or alternation with one or more other antivirally effective agents, optionally in a pharmaceutically acceptable carrier or diluent;

g) use of an agent, as described herein, or its pharmaceutically acceptable salt or prodrug thereof in medical therapy, i.e. as an antiviral or antiproliferative agent;

h) use of an agent, as described herein, or its pharmaceutically acceptable salt or prodrug thereof, optionally in a pharmaceutically acceptable carrier or diluent, for the treatment or prophylaxis of abnormal cellular proliferation and/or viral infection, and in particular a Flaviviridae infection, such as an HCV or BVDV infection, in a host, and in particular in a human;

i) use of an agent, as described herein, or its pharmaceutically acceptable salt or prodrug thereof, optionally in a pharmaceutically acceptable carrier or diluent, for the treatment or prophylaxis of abnormal cellular proliferation and/or viral infection, and in particular a Flaviviridae infection, such as an HCV or BVDV infection, in a host, and in particular in a human, in combination or alternation with one or more other antivirally effective agents;

j) use of an agent, as described herein, or its pharmaceutically acceptable salt or prodrug thereof in the manufacture of a medicament for use in medical therapy, i.e. as an antiviral or antiproliferative agent;

k) use of an agent, as described herein, or its pharmaceutically acceptable salt or prodrug thereof, optionally in a pharmaceutically acceptable carrier or diluent, in the manufacture of a medicament for the treatment or prophylaxis of abnormal cellular proliferation and/or viral infection, and in particular a Flaviviridae infection, such as an HCV or BVDV infection, in a host, and in particular in a human; and

l) use of an agent, as described herein, or its pharmaceutically acceptable salt or prodrug thereof, optionally in a pharmaceutically acceptable carrier or diluent, in the manufacture of a medicament for the treatment or prophylaxis of abnormal cellular proliferation and/or viral infection, and in particular a Flaviviridae infection, such as an HCV or BVDV infection, in a host, and in particular in a human, in combination or alternation with one or more other antivirally effective agents.

In an additional embodiment, a method for the treatment or prophylaxis of a host, such as a mammal and in particular a human, having a virus-associated disorder which comprises administering to the mammal a pharmaceutically effective amount of one of the described antiviral agents or their pharmaceutically acceptable salts or prodrugs thereof, is provided.

In one particular embodiment, a method for the treatment or prophylaxis of a Flaviviridae infection, including HCV and BVDV infection, in a host, that includes administering an antivirally effective amount of one of the described antiviral agents or its pharmaceutically acceptable salt or prodrug thereof, optionally in a pharmaceutically acceptable carrier or diluent thereof, is provided.

In another particular embodiment, a method for treatment or prophylaxis of a Flaviviridae infections, including HCV and BVDV infection, in a host, that includes administering an antivirally effective amount of one of the described antiviral agents or its pharmaceutically acceptable salt or prodrug thereof, optionally in a pharmaceutically acceptable carrier or diluent, in combination or alternation with one or more other effective agent is provided.

In an additional embodiment, the use of an effective amount of one of the described antiviral agents or their pharmaceutically acceptable salts or prodrugs thereof, for the treatment or prophylaxis of a host, such as a mammal and in particular a human, having a virus-associated disorder is provided.

In one particular embodiment, the use of an antivirally effective amount of one of the described antiviral agents or its pharmaceutically acceptable salt or prodrug thereof, optionally in a pharmaceutically acceptable carrier or diluent thereof, for the treatment or prophylaxis of a Flaviviridae infection, including HCV and BVDV infection, in a host is provided.

In another particular embodiment, the use of an antivirally effective amount of one of the described antiviral agents or its pharmaceutically acceptable salt or prodrug thereof, optionally in a pharmaceutically acceptable carrier or diluent, in combination or alternation with one or more other effective agent for the treatment or prophylaxis of a Flaviviridae infection, including HCV and BVDV infection, in a host is provided.

In an additional embodiment, the use of an effective amount of one of the described antiviral agents or their pharmaceutically acceptable salts or prodrugs thereof, in the manufacture of a medicament for the treatment or prophylaxis of a host, such as a mammal and in particular a human, having a virus-associated disorder is provided.

In one particular embodiment, the use of an antivirally effective amount of one of the described antiviral agents or its pharmaceutically acceptable salt or prodrug thereof, optionally in a pharmaceutically acceptable carrier or diluent thereof, in the manufacture of a medicament for the treatment or prophylaxis of a Flaviviridae infection, including HCV and BVDV infection, in a host is provided.

In another particular embodiment, the use of an antivirally effective amount of one of the described antiviral agents or its pharmaceutically acceptable salt or prodrug thereof, optionally in a pharmaceutically acceptable carrier or diluent, in combination or alternation with one or more other effective agent in the manufacture of a medicament for the treatment or prophylaxis of a Flaviviridae infection, including HCV and BVDV infection, in a host is provided.

In one embodiment, the antiviral agent has an EC₅₀ (effective concentration to achieve 50% viral inhibition) when tested in an appropriate cell-based assay, of less than 15 micromolar, and more particularly, less than 10 or 5 micromolar.

While the disclosed compounds are illustrated in β-D or β-L configuration, it should be understood that any of the compounds described herein can alternatively be used in the opposite stereoconfiguration, or a mixture thereof. In a preferred embodiment, the selected optical isomer is used in substantially pure form (i.e. approximately 95% pure or greater). For example, any compound illustrated herein in a β-D configuration can also be administered in the β-L configuration, and vice versa.

Flaviviruses included within the scope of this invention are discussed generally in Fields Virology, Editors: Fields, B. N., Knipe, D. M., and Howley, P. M., Lippincott-Raven Publishers, Philadelphia, Pa., Chapter 31, 1996. Specific flaviviruses include, without limitation: Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, Royal Farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, West Nile, Yaounde, Yellow fever, and Zika.

Pestiviruses included within the scope of this invention are discussed generally in Fields Virology, Editors: Fields, B. N., Knipe, D. M., and Howley, P. M., Lippincott-Raven Publishers, Philadelphia, Pa., Chapter 33, 1996. Specific pestiviruses include, without limitation: bovine viral diarrhea virus (“BVDV”), classical swine fever virus (“CSFV,” also called hog cholera virus), and border disease virus (“BDV”).

During the course of the development of any RNA-dependent RNA polymerase (RDRP) antiviral agent, the inhibitory effect on DNA-dependent RNA polymerase (DDRP) will need to be considered and measured in a sensitive and reproducible way. Therefore, in one embodiment of the invention, a method is disclosed that allows measuring of small differences in the intra-cellular quantities of the transcripts derived from the different DDRPs and RDRP simultaneously. The method is based upon the single-tube RT-PCR using real-time fluorescent technology of the RNA products derived from the different polymerase enzyme activities.

### BRIEF DESCRIPTION OF THE FIGURES

FIG. 1 is an illustration of the de novo synthesis of guanine nucleotides via inosine monophosphate dehydrogenase (IMPDH), mediated by the cofactor nicotinamide adenine dinucleotide (NAD).

FIG. 2 is an illustration of azole nucleoside isomers that are generated during a condensation reaction as described in Scheme 6.

FIG. 3 is a graphical depiction of the effect of increased concentration of Ribavirin on cell viability, i.e. the cytopathic effect of Ribavirin on MDBK cells.

FIG. 4 is a graphical depiction of the toxic effects of C2-MAD on Balb/c mice over a ten day treatment period. The line indicated by circular points represents the effect of water; the square 10 mg/kg/day, the triangle 30 mg/kg/day and the diamond 60 mg/kg/day. As indicated, the compound does not contribute to significant weight gain or loss.

FIG. 5 is an illustration of the increase in plaque forming units with increasing concentration of bovine viral diarrhea virus (“BVDV”) in cell culture as described in Example 15. FIG. 5 establishes that the method of Example 15 provides reliable quantification of BVDV over a four log PFU/mL of virus.

FIG. 6 is an illustration of the BVDV replication cycle in MDBK cells to determine the optimal harvesting time (in hours post infection versus the log of plaque forming units (“PFU”), i.e. 22 hours after infection, which roughly corresponds to approximately one replication cycle, where the amount of virus produced is equal to the amount of virus inoculated into the cell, as described in Example 16.

FIG. 7 is a line graph depicting the inhibition of viral production of various concentrations of ribavirin (RIB) and interferon (IFN) relative to no drug over a 4 day incubation period.

FIG. 8 is a bar chart graph showing the ability of certain test compounds to inhibit the production of plaque forming units during one replication cycle, as described in Example 17 against BVDV.

FIG. 9 is a dose-response curve for C2-MAD relative to ribavirin (Rib) and Tiazofurin. When comparing intracellular viral RNA reduction, ribavirin is more effective than C2-MAD, which is more effective than Tiazofurin. When comparing supernatant viral RNA, a similar pattern is found.

FIG. 10 is a bar chart graph depicting the competitive effects of exogenous guanosine. The antiviral effect of all the compounds depicted was diminished or reversed with the addition of guanosine, indicating that all compounds are competitive inhibitors of IMPDH.

### DETAILED DESCRIPTION OF THE INVENTION

The present invention provides compounds, compositions and methods for the treatment or prophylaxis of an immunological disorder, abnormal cellular proliferation or viral infection, and in particular a Flaviviridae infection, including an HCV or a BVDV infection, in a host comprising administering an effective agent of the present invention. Preferably, the effective agent selectively inhibits inosine monophosphate dehydrogenase (IMPDH) and/or its cofactor, nicotinamide adenine dinucleotide (NAD). The agent can inhibit IMPDH by acting as a substrate analog (inosine monophosphate (IMP) analog), blocking the NAD binding site, or acting as an NAD analog. In one embodiment of the present invention, the effective antiviral agent does not require in vitro or in vivo activation to be an inhibitor.

In an alternate embodiment of the present invention, the effective antiviral agent requires in vitro or in vivo activation to become an inhibitor. Non-limiting examples of necessary activation include phosphorylation, such as with ribavarin and mizoribine wherein phosphorylation to the monophosphate is necessary to inhibit IMPDH, or conversion into the adenine dinucleotide, as with tiazofurin and benzamide riboside wherein conversion into the adenine dinucleotide TAD and BAD respectively is necessary to inhibit IMPDH.

The present invention includes at least the following features:

a) a compound for the treatment or prophylaxis of a Flaviviridae infection;

b) a process for the preparation of the effective agents described herein, their pharmaceutically acceptable salts and prodrugs thereof;

c) a pharmaceutical composition that includes an effective amount of an agent as described herein, or its pharmaceutically acceptable salt or prodrug thereof together with a pharmaceutically acceptable carrier or diluent according to the present invention for the treatment or prophylaxis of abnormal cellular proliferation and/or viral infection, and in particular a Flaviviridae infection, such as an HCV or BVDV infection, in a host, and in particular in a human;

d) a pharmaceutical composition that includes an effective amount of an agent as described herein, or its pharmaceutically acceptable salt or prodrug thereof in combination with one or more other antivirally effective agents, optionally in a pharmaceutically acceptable carrier or diluent according to the present invention for the treatment or prophylaxis of abnormal cellular proliferation and/or viral infection, and in particular a Flaviviridae infection, such as an HCV or BVDV infection, in a host, and in particular in a human;

e) a method for the treatment or prophylaxis of abnormal cellular proliferation and/or viral infection, and in particular a Flaviviridae infection, including hepatitis C virus or BVDV infection, in a host, and in particular a human, comprising administering an effective amount of an agent as described herein, or its pharmaceutically acceptable salt or prodrug thereof, optionally in a pharmaceutically acceptable carrier or diluent;

f) a method for the treatment or prophylaxis of abnormal cellular proliferation and/or viral infection, and in particular a Flaviviridae infection, including hepatitis C virus or BVDV infection, in a host, an in particular a human, comprising administering an effective amount of an agent as described herein, or its pharmaceutically acceptable salt or prodrug thereof in combination or alternation with one or more other antivirally effective agents, optionally in a pharmaceutically acceptable carrier or diluent;

g) use of an agent, as described herein, or its pharmaceutically acceptable salt or prodrug thereof in medical therapy, i.e. as an antiviral or antiproliferative agent;

h) use of an agent, as described herein, or its pharmaceutically acceptable salt or prodrug thereof, optionally in a pharmaceutically acceptable carrier or diluent, for the treatment or prophylaxis of abnormal cellular proliferation and/or viral infection, and in particular a Flaviviridae infection, such as an HCV or BVDV infection, in a host, and in particular in a human;

i) use of an agent, as described herein, or its pharmaceutically acceptable salt or prodrug thereof, optionally in a pharmaceutically acceptable carrier or diluent, for the treatment or prophylaxis of abnormal cellular proliferation and/or viral infection, and in particular a Flaviviridae infection, such as an HCV or BVDV infection, in a host, and in particular in a human, in combination or alternation with one or more other antivirally effective agents;

j) use of an agent, as described herein, or its pharmaceutically acceptable salt or prodrug thereof in the manufacture of a medicament for use in medical therapy, i.e. as an antiviral or antiproliferative agent;

k) use of an agent, as described herein, or its pharmaceutically acceptable salt or prodrug thereof, optionally in a pharmaceutically acceptable carrier or diluent, in the manufacture of a medicament for the treatment or prophylaxis of abnormal cellular proliferation and/or viral infection, and in particular a Flaviviridae infection, such as an HCV or BVDV infection, in a host, and in particular in a human; and

l) use of an agent, as described herein, or its pharmaceutically acceptable salt or prodrug thereof, optionally in a pharmaceutically acceptable carrier or diluent, in the manufacture of a medicament for the treatment or prophylaxis of abnormal cellular proliferation and/or viral infection, and in particular a Flaviviridae infection, such as an HCV or BVDV infection, in a host, and in particular in a human, in combination or alternation with one or more other antivirally effective agents.

In an additional embodiment, a method for the treatment or prophylaxis of a host, such as a mammal and in particular a human, having a virus-associated disorder which comprises administering to the mammal a pharmaceutically effective amount of one of the described antiviral agents or their pharmaceutically acceptable salts or prodrugs thereof, is provided.

In one particular embodiment, a method for the treatment or prophylaxis of a Flaviviridae infection, including HCV and BVDV infection, in a host, that includes administering an antivirally effective amount of one of the described antiviral agents or its pharmaceutically acceptable salt or prodrug thereof, optionally in a pharmaceutically acceptable carrier or diluent thereof, is provided.

In another particular embodiment, a method for treatment or prophylaxis of a Flaviviridae infections, including HCV and BVDV infection, in a host, that includes administering an antivirally effective amount of one of the described antiviral agents or its pharmaceutically acceptable salt or prodrug thereof, optionally in a pharmaceutically acceptable carrier or diluent, in combination or alternation with one or more other effective agent is provided.

In an additional embodiment, the use of an effective amount of one of the described antiviral agents or their pharmaceutically acceptable salts or prodrugs thereof, for the treatment or prophylaxis of a host, such as a mammal and in particular a human, having a virus-associated disorder is provided.

In one particular embodiment, the use of an antivirally effective amount of one of the described antiviral agents or its pharmaceutically acceptable salt or prodrug thereof, optionally in a pharmaceutically acceptable carrier or diluent thereof, for the treatment or prophylaxis of a Flaviviridae infection, including HCV and BVDV infection, in a host is provided.

In another particular embodiment, the use of an antivirally effective amount of one of the described antiviral agents or its pharmaceutically acceptable salt or prodrug thereof, optionally in a pharmaceutically acceptable carrier or diluent, in combination or alternation with one or more other effective agent for the treatment or prophylaxis of a Flaviviridae infection, including HCV and BVDV infection, in a host is provided.

In an additional embodiment, the use of an effective amount of one of the described antiviral agents or their pharmaceutically acceptable salts or prodrugs thereof, in the manufacture of a medicament for the treatment or prophylaxis of a host, such as a mammal and in particular a human, having a virus-associated disorder is provided.

In one particular embodiment, the use of an antivirally effective amount of one of the described antiviral agents or its pharmaceutically acceptable salt or prodrug thereof, optionally in a pharmaceutically acceptable carrier or diluent thereof, in the manufacture of a medicament for the treatment or prophylaxis of a Flaviviridae infection, including HCV and BVDV infection, in a host is provided.

In another particular embodiment, the use of an antivirally effective amount of one of the described antiviral agents or its pharmaceutically acceptable salt or prodrug thereof, optionally in a pharmaceutically acceptable carrier or diluent, in combination or alternation with one or more other effective agent in the manufacture of a medicament for the treatment or prophylaxis of a Flaviviridae infection, including HCV and BVDV infection, in a host is provided.

I. Active Compounds

In one embodiment of the invention, the active compound is of the following formula (I):

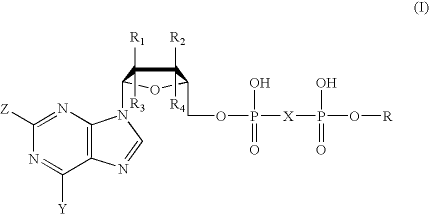

or its pharmaceutically acceptable salt or prodrug thereof; wherein

R is

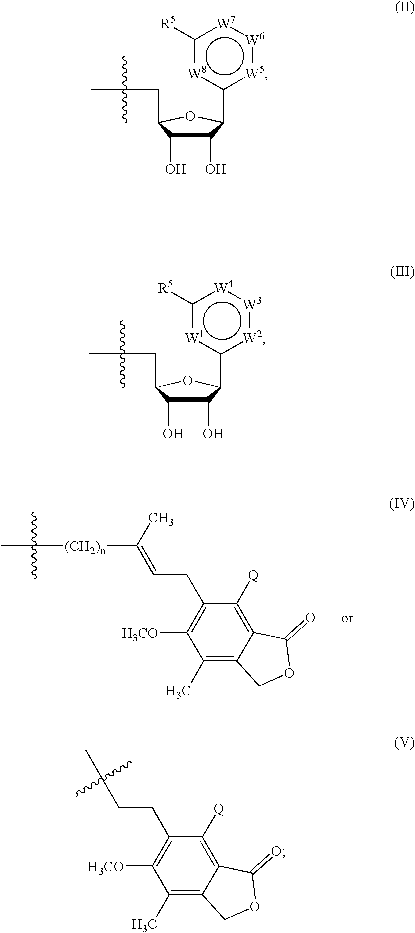

X is oxygen, sulfur, methylene, monofluoromethylene or difluoromethylene;

Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;

Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;

W¹-W⁴ are same or different, and independently methyne (—CH═), azomethyne (—N═) or sulfur;

W⁵-W⁸ are same or different, and independently methyne (—CH═) or azomethyne (—N═);

R¹, R², R³ and R⁴ are independently hydrogen, hydroxyl or fluorine;

R⁵ is halogen (F, Cl, Br, I), CN, CONH₂, CO₂Me, CO₂Et or CO₂H; and

R⁶, R⁷, R⁸ and R⁹ are independently a lower alkane or alkene of 1, 2, 3, 4, 5 or 6 carbons or aryl or aralkyl such as unsubstituted or substituted phenyl or benzyl.

In one embodiment of the present invention, the compound of the general formula (I) is specifically not tiazole-4-carboxamide adenine dinucleotide (TAD) or benzamide adenine dinucleotide (BAD).

The structures of compounds with general formula (I) are represented by (but not limited to) structures provided below:

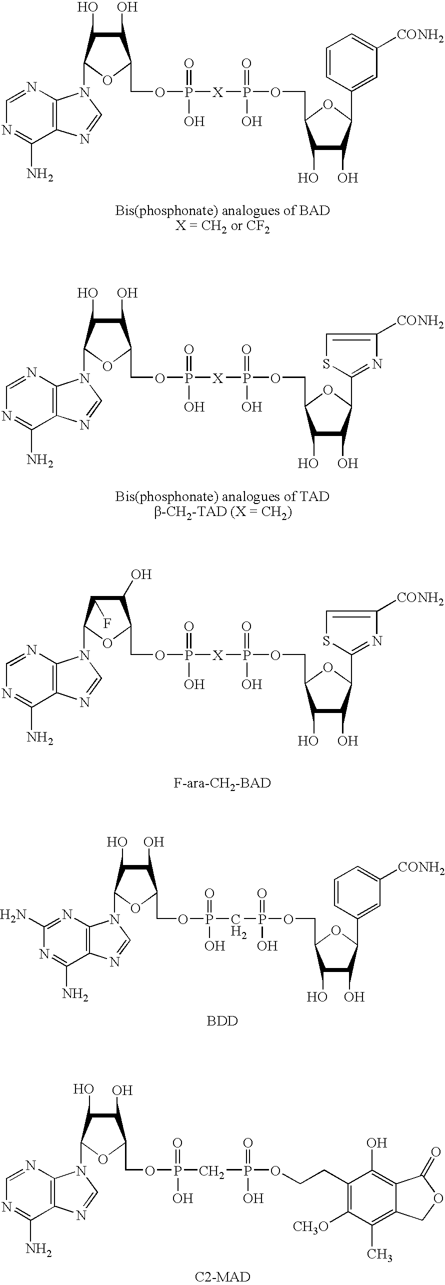

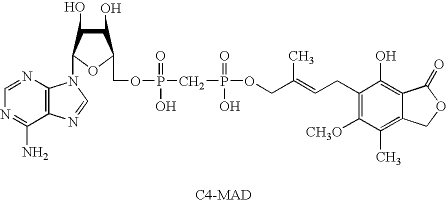

In addition, truncated compounds chemically modified as discussed below in order to improve their activity, and in particular antiviral activity, and/or decrease their toxicity are also provided. In particular, the present invention also provides compounds wherein the molecular structures are composed of parts (fragments) of compounds of formula (I), e.g. C2-, C4-, and C6-mycophenolic alcohols, as well as mycophenolic alcohols modified by simple oxidation to the corresponding aldehyde or carboxylic acid derivatives, for example, the following:

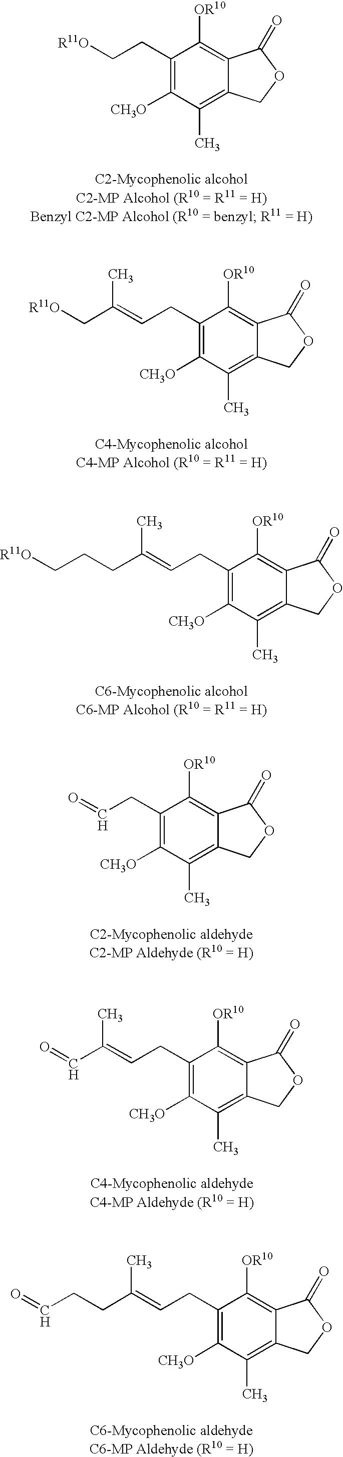

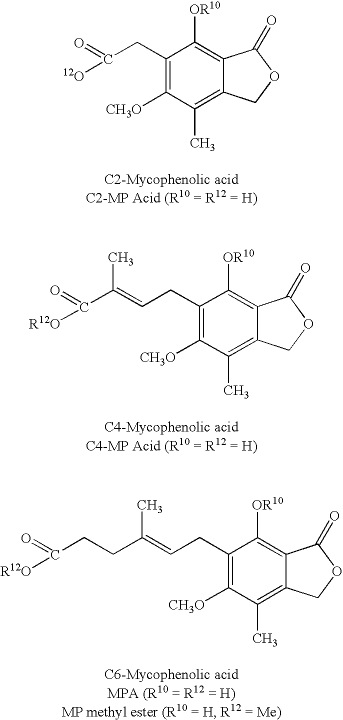

or its pharmaceutically acceptable salt or prodrug, wherein each R¹⁰ and R¹¹ is independently hydrogen, alkyl, acyl, benzyl or methoxymethyl (MOM) group, and each R¹² is independently hydrogen, alkyl or aryl.

These compounds can be modified by replacement of the alcohol, aldehyde or carboxyl group with the corresponding sulfonyl or phosphoryl functional group.

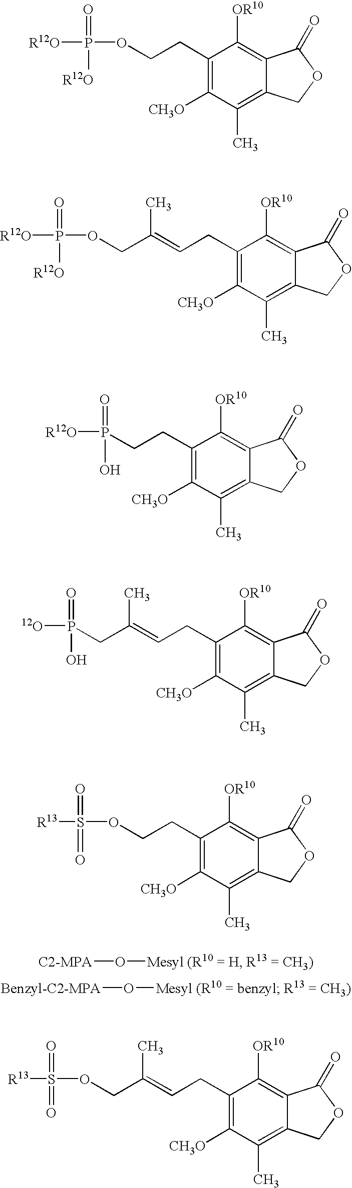

or its pharmaceutically acceptable salt or prodrug thereof, wherein R¹⁰ and R¹² are as defined above, and R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl.

The parent mycophenolic alcohols can also be reduced to the corresponding alkyl derivatives or dehydrated to the alkenyl or alkynyl derivatives. Non-limiting examples include the following:

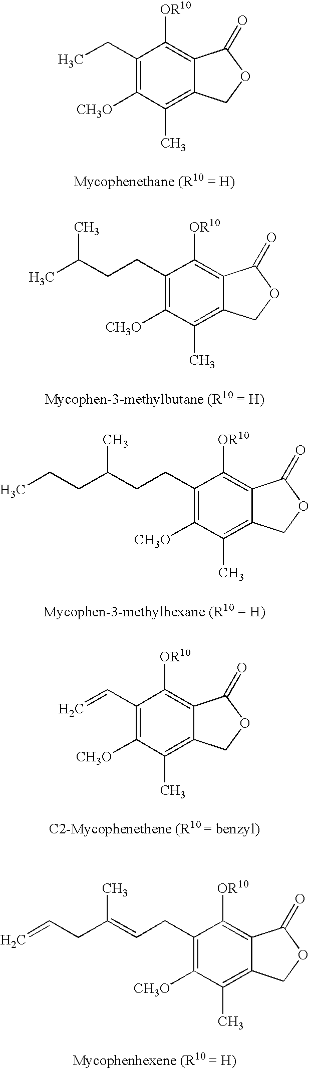

or its pharmaceutically acceptable salt or prodrug thereof, wherein R¹⁰ is as defined above.

Truncated compounds can be composed of larger fragments such as bis(phosphonate) analogues of mycophenolic alcohols. These compounds may be modified by coupling with another mycophenolic alcohol derivative to give a dimer, e.g. bis-C2MPAlcbis(phosphonate), such as the following:

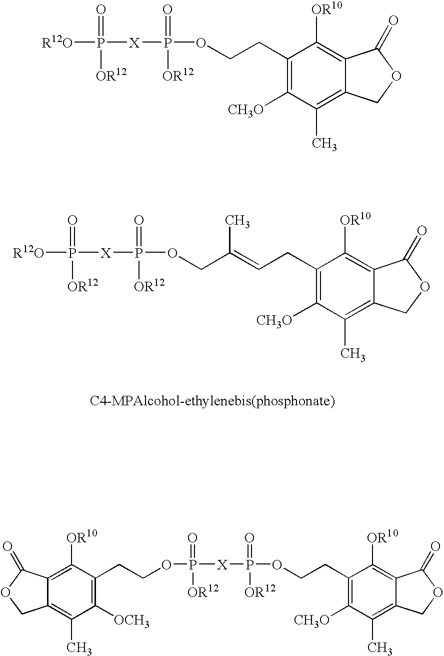

or its pharmaceutically acceptable salt or prodrug thereof, wherein X, R¹⁰ and R¹² are as defined above.

The truncated compounds can also be nucleosides such as tiazofurin, benzamide riboside, C-nicotinamide riboside or F-ara-purines (such as F-ara-A).

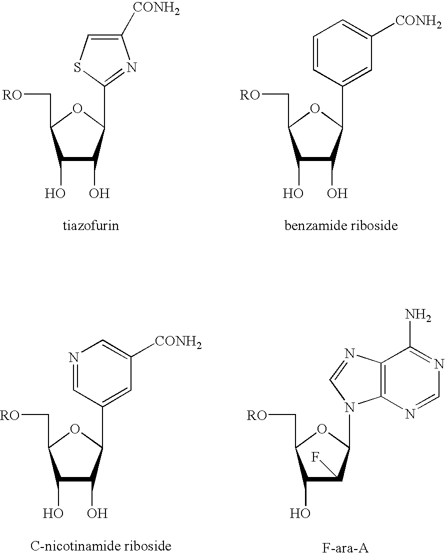

or its pharmaceutically acceptable salt or prodrug, wherein R can be hydrogen, acyl or silyl.

In a preferred embodiment of the invention, the compounds can be administered in the form of an ether lipid. Alternatively, the phosphonates and phosphoryls can be administered in the form of stabilized phosphate or phospholipid.

In one embodiment, the antiviral agent has an EC₅₀ (effective concentration to achieve 50% viral inhibition) when tested in an appropriate cell-based assay, of less than 15 micromolar, and more particularly, less than 10 or 5 micromolar

II. Stereoisomerism and Polymorphism

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

In one embodiment of the invention, the compounds are provided in substantially pure form (i.e. approximately 95% pure or greater).

Optically active forms of the compounds can be prepared using any method known in the art, including by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including simulated moving bed chromatography, is used in one embodiment. A wide variety of chiral stationary phases are commercially available.

III. Definitions

The term “alkyl,” as used herein, unless otherwise specified, refers to a saturated straight, branched, or cyclic, primary, secondary, or tertiary hydrocarbon, including but not limited to those of C₁ to C₁₆, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrazine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl,” as used herein, and unless otherwise specified, refers to a C₁ to C₄ saturated straight, branched, or if appropriate, a cyclic (for example, cyclopropyl) alkyl group, including both substituted and unsubstituted forms.

As used herein, the term “substantially free of” or “substantially in the absence of” refers to a nucleoside composition that includes at least 95% to 98% by weight, and even more preferably 99% to 100% by weight, of the designated enantiomer of that nucleoside. In a preferred embodiment, in the methods and compounds of this invention, the compounds are substantially free of enantiomers.

Similarly, the term “isolated” refers to a compound composition that includes at least 95% to 98% by weight, and even more preferably 99% to 100% by weight, of the compound, the remainder comprising other chemical species or enantiomers.

The term “enantiomerically enriched” is used throughout the specification to describe a compound which includes at least about 95%, preferably at least 96%, more preferably at least 97%, even more preferably, at least 98%, and even more preferably at least about 99% or more of a single enantiomer of that compound. When a nucleoside of a particular configuration (D or L) is referred to in this specification, it is presumed that the nucleoside is an enantiomerically enriched nucleoside, unless otherwise stated.

The term “host,” as used herein, refers to a unicellular or multicellular organism in which the virus can replicate, including cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans. Alternatively, the term “host,” as used herein, refers to a multicellular organism in which proliferative disorders can occur, including animals, and preferably a human. Alternatively, the host is any abnormally proliferating cell, whose replication or function can be altered by the compounds of the present invention. The term host specifically refers to any cell line that abnormally proliferates, either from natural or unnatural causes (for example, from genetic mutation or genetic engineering, respectively), and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

The term “pharmaceutically acceptable prodrug” is used throughout the specification to describe any pharmaceutically acceptable form (such as an ester, phosphate ester or salt of an ester or a related group) of a disclosed compound which, upon administration to a patient, provides the active parent compound. Pharmaceutically acceptable prodrugs, for example, refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. The compounds of this invention either possess antiviral activity such as against Flaviviridae viruses and/or antiproliferative activity, or are metabolized to a compound that exhibits such activity.

IV. Pharmaceutically Acceptable Salts and Prodrugs

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the compound. For example, one or more hydrogens on the phosphonate moiety can be replaced with alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed compounds to achieve a desired effect.

The active compounds can also be provided as phosphoether lipids or ether lipids, as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the compound include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

V. Pharmaceutical Compositions

Pharmaceutical compositions that include a β-D or the β-L stereoisomer can be prepared that include the above-described compound or its salt or prodrug in a therapeutically effective amount, optionally in combination with a pharmaceutically acceptable additive, carrier or excipient for treating any of the conditions described herein, including a Flaviviridae infection. The therapeutically effective amount may vary with the infection or condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in orally administrable form, but formulations may be administered via parenteral, intravenous, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification, etc.).

In certain pharmaceutical dosage forms, the prodrug forms of the compound, especially including acylated (including acetylated or other) and ether derivatives, phosphate esters, stabilized phosphates, and various salt forms of the present compounds, are preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of any of the conditions described herein, including a Flaviviridae infection (such as an HCV infection).

The amount of compound included within therapeutically active formulations, according to the present invention, is an effective amount for treating any of the conditions described herein, including a Flaviviridae infection. In general, a therapeutically effective amount of the present compound in pharmaceutical dosage form usually ranges from about 0.1 mg/kg to about 100 mg/kg or more, depending upon the compound used, the condition or infection treated and the route of administration. For purposes of the present invention, a prophylactically or preventively effective amount of the compositions, according to the present invention, falls within the same concentration range as set forth above for therapeutically effective amount and is usually the same as a therapeutically effective amount.

Administration of the active compound may range from continuous (intravenous drip) to several oral administrations per day (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. In particular, this may be appropriate for the delivery of free nucleosides, acyl nucleosides, phosphate ester prodrug forms as well as the bisphosphonate compounds according to the present invention.

In particularly preferred embodiments according to the present invention, the compounds and compositions are used to treat, prevent or delay the onset of any of the conditions described herein, including a Flaviviridae infection. Preferably, to treat, prevent or delay the onset of the condition, the compositions will be administered in oral dosage form in amounts ranging from about 250 micrograms up to about 1 gram or more at least once a day, preferably, or up to four times a day. The present compounds are preferably administered orally, but may be administered parenterally, topically or in suppository form.

The compounds according to the present invention, because of their low toxicity to host cells in certain instances, may be advantageously employed prophylactically to prevent any of the conditions described herein, including a Flaviviridae infection or to prevent the occurrence of clinical symptoms associated with the condition. Thus, the present invention also encompasses methods for the prophylactic treatment of any of the conditions described herein, including a Flaviviridae infection. In this aspect, according to the present invention, the present compositions are used to prevent or delay the onset of any of the conditions described herein, including a Flaviviridae infection (including HCV infection). This prophylactic method comprises administration to a patient in need of such treatment, or who is at risk for the development of any of the conditions described herein, including a Flaviviridae infection, and in particular an HCV infection, an amount of a compound according to the present invention effective for alleviating, preventing or delaying the onset of the condition. It is preferred in this aspect of the present invention that the compound that is used is maximally effective against the condition and exhibits a minimum of toxicity to the patient. In the case of Flaviviridae infection, compounds according to the present invention that may be used to treat these disease states may be administered within the same dosage range for therapeutic treatment (i.e., about 250 micrograms up to 1 gram or more from one to four times per day for an oral dosage form) as a prophylactic agent to prevent the proliferation of a Flaviviridae infection, or alternatively, to prolong the onset of a Flaviviridae infection, which manifests itself in clinical symptoms.

In addition, compounds according to the present invention can be administered in combination or alternation with one or more antiviral, anti-HBV, anti-HCV or anti-herpetic agent or interferon, anti-cancer, antiproliferative or antibacterial agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect. ps VI. Combination and Alternation Therapies for the Treatment of HCV Infection

It has been recognized that drug-resistant variants of HCV can emerge after prolonged treatment with an antiviral agent. Drug resistance most typically occurs by mutation of a gene that encodes for an enzyme used in the viral replication cycle, and most typically in the case of HCV, the RNA-dependent-RNA polymerase. Recently, it has been demonstrated that the efficacy of a drug against HCV infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy. In general, combination therapy is typically preferred over alternation therapy because it induces multiple simultaneous stresses on the virus.

Examples of agents that have been identified as active against the hepatitis C flavivirus, and thus can be used in combination or alternation with one or more agents of formula (I) to (V), or truncated and modified forms thereof, include:

(1) interferon and ribavirin (Battaglia, A. M. et al. Ann. Pharmacother. 2000, 34, 487; Berenguer, M. et al. Antivir. Ther. 1998, 3 (Suppl. 3), 125);

(2) Substrate-based NS3 protease inhibitors (Attwood et al. PCT WO 98/22496, 1998; Attwood et al. Antiviral Chemistry and Chemotherapy 1999, 10, 259,; Attwood et al. German Patent Pub. DE 19914474; Tung et al. PCT WO 98/17679), including alphaketoamides and hydrazinoureas, and inhibitors that terminate in an electrophile such as a boronic acid or phosphonate (Llinas-Brunet et. al. PCT WO 99/07734).

(3) Non-substrate-based inhibitors such as 2,4,6-trihydroxy-3-nitro-benzamide derivatives (Sudo K. et al., Biochemical and Biophysical Research Communications, 1997, 238, 643 and Sudo K. et al. Antiviral Chemistry and Chemotherapy 1998, 9, 186), including RD3-4082 and RD3-4078, the former substituted on the amide with a 14 carbon chain and the latter processing a para-phenoxyphenyl group;

(4) Thiazolidine derivatives that show relevant inhibition in a reverse-phase HPLC assay with an NS3/4A fusion protein and NS5A/5B substrate (Sudo K. et al. Antiviral Research 1996, 32, 9), especially compound RD-1-6250, possessing a fused cinnamoyl moiety substituted with a long alkyl chain, RD4 6205 and RD4 6193;

(5) Thiazolidines and benzanilides identified in Kakiuchi N. et al. J. EBS Letters 421, 217 and Takeshita N. et al. Analytical Biochemistry 1997, 247, 242;

(6) A phenanthrenequinone possessing activity against HCV protease in a SDS-PAGE and autoradiography assay isolated from the fermentation culture broth of Streptomyces sp., Sch 68631 (Chu M. et al. Tetrahedron Letters 1996, 37, 7229), and Sch 351633, isolated from the fungus Penicillium griscofuluum, which demonstrates activity in a scintillation proximity assay (Chu M. et al., Bioorganic and Medicinal Chemistry Letters 9, 1949);

(7) Selective NS3 inhibitors based on the macromolecule elgin c, isolated from leech (Qasim M. A. et al. Biochemistry 1997, 36,1598);

(8) HCV helicase inhibitors (Diana G. D. et al., U.S. Pat. No. 5,633,358 and Diana G. D. et al. PCT WO 97/36554);

(9) HCV polymerase inhibitors such as nucleotide analogues, gliotoxin (Ferrari R. et al. Journal of Virology 1999, 73, 1649), and the natural product cerulenin (Lohmann V. et al. Virology 1998, 249, 108);

(10) Antisense phosphorothioate oligodeoxynucleotides (S-ODN) complementary to sequence stretches in the 5′ non-coding region (NCR) of the HCV (Alt M. et al. Hepatology 1995, 22, 707), or nucleotides 326-348 comprising the 3′ end of the NCR and nucleotides 371-388 located in the core coding region of the HCV RNA (Alt M. et al. Archives of Virology 1997, 142, 589 and Galderisi U. et al., Journal of Cellular Physiology 1999, 81:2151);

(11) Inhibitors of IRES-dependent translation (Ikeda N et al. Japanese Patent Pub. JP-08268890; Kai Y. et al. Japanese Patent Pub. JP-10101591);

(12) Nuclease-resistant ribozymes. (Maccjak D. J. et al., Hepatology 1999, 30, abstract 995); and

(13) Other miscellaneous compounds including 1-amino-alkylcyclohexanes (Gold et al. U.S. Pat. No. 6,034,134), alkyl lipids (Chojkier et al U.S. Pat. No. 5,922,757), vitamin E and other antioxidants (Chojkier et al. U.S. Pat. No. 5,922,757), squalene, amantadine, bile acids (Ozeki et al. U.S. Pat. No. 5,846,964), N-(phosphonoacetyl)-L-aspartic acid, (Diana et al. U.S. Pat. No. 5,830,905), benzenedicarboxamides (Diana et al. U.S. Pat. No. 5,633,388), polyadenylic acid derivatives (Wang et al. U.S. Pat. No. 5,496,546), 2′,3′-dideoxyinosine (Yarchoan et al. U.S. Pat. No. 5,026,687), and benzimidazoles (Colacino et al. U.S. Pat. No. 5,891,874).

VII. Synthetic Protocol

Compounds of formula (I) can be synthesized by the tetraphosphonate bicyclic trisanhydride method (Pankiewicz et al., WO 98/15563). Compounds wherein the R group is of the formula (II) or (III) and their derivatives can be synthesized by modified literature procedures. Compounds of formulae (IV) and (V) and their derivatives can be prepared from mycophenolic acid.

Synthesis of Compounds of Formula [I] and their Derivatives:

A β-D-purine nucleoside 5′-methylenebis(phosphonate) (1, Scheme 1) is treated with 2-7 molar excess, preferably 3-4 molar excess, of dehydrating agent such as dicyclohexylcarbo-diimide (DCC) or other carbodiimide, such as diisopropyl-carbodiimide, 1-[3-(dimethylamino)-propyl]-3-ethylcarbodiimide hydrochloride, 1-[3-(dimethylamino)-propyl]-3-ethylcarbodiimide methiodide and the like, in anhydrous solvent, such as pyridine, pycoline, N,N-dimethyl-formamide (DMF), dimethylsulfoxide (DMSO), hexamethylene-phosphoric triamide, preferably pyridine, at a temperature of from 0° C. to 100° C., preferably between 20° C. to 60° C. for a period of from 1 hour to 24 hours, preferably 4-8 hours. The progress of the reaction can be monitored by ³¹P NMR. Formation of P¹,P⁴-disubstituted methylenebis(phosphonic) anhydride 2 is observed first, but the relatively simple signals of 2 disappears with simultaneous dehydration between P¹ and P³ forming 3, which is further dehydrate between P² and P⁴ to give the bicyclic trisanhydride intermediate 4 (three characteristic multi-signal resonances at −0.5-2.0 ppm, at 6.0-8.0 ppm and at 12.8-17.6 ppm). When the formation of 4 is completed, an alcohol (R—OH) is added to the mixture and heated to a temperature of from 35° C. to 100° C., preferably from 60° C. to 70° C., for a period of from 1 hour to 48 hours, preferably from 18 to 24 hours. Nucleophilic attack by the alcohol occurs at P-2 and P-3 to form the open-chain intermediate 5. The reaction can be followed by ³¹P NMR spectroscopy, which shows much simpler two broad signals centered at 8 and 18 ppm. Intermediate 5 undergoes rapid hydrolysis upon addition of water to the reaction mixture forming the P¹,P²-disubstituted methylenebis(phosphonate) product with structure [I]. After concentration of the mixture in vacuo, the residue is chromatographed on a preparative HPLC column to give the desired product with structure [I].

The β-L counterpart of [I] also can be synthesized by using the β-L purine nucleoside 5′-methylenebis(phosphonate) counterpart of (1).

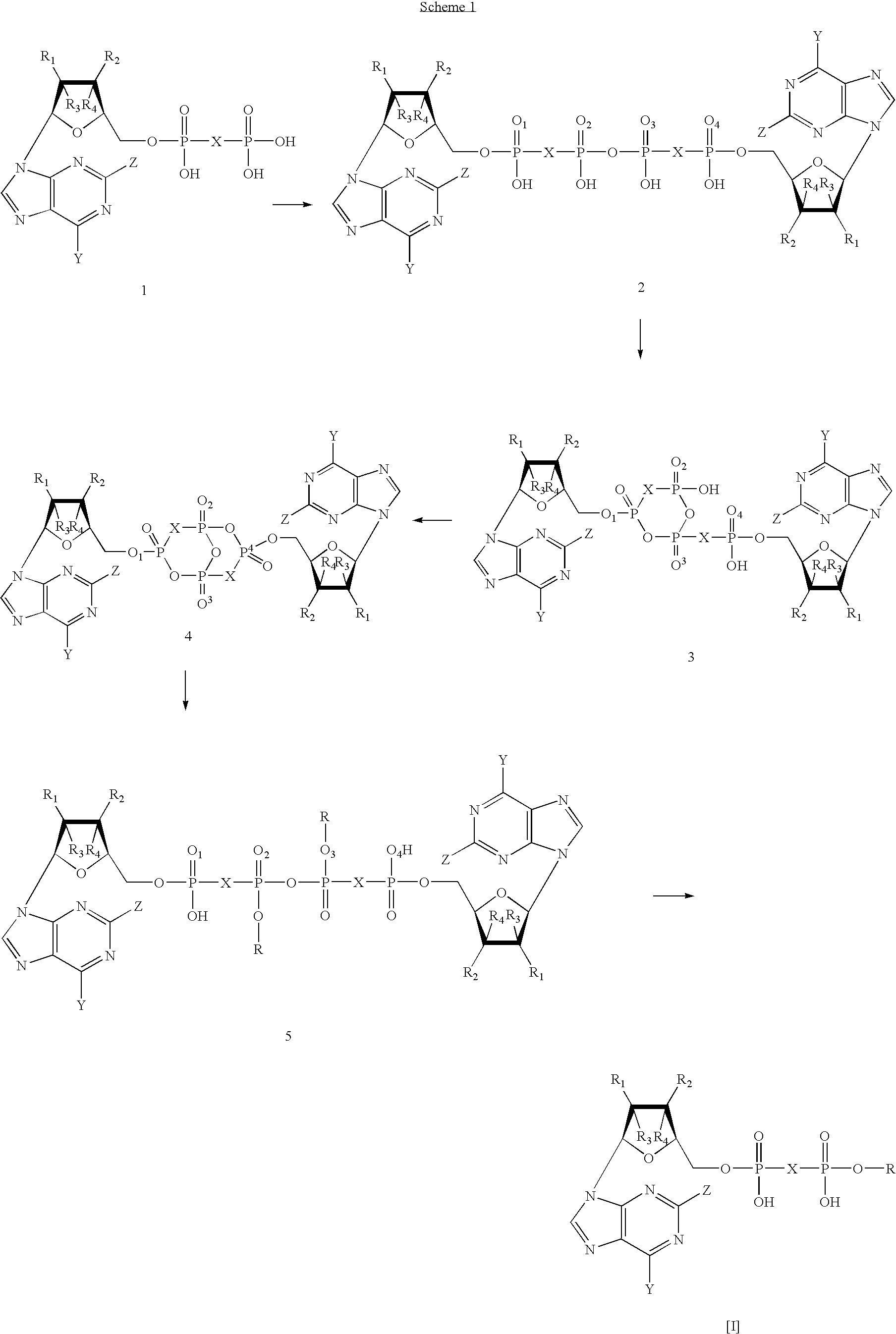

Alternatively, an alcohol synthon, ROH, is converted into methylenebis-(phosphonate) 6 (Scheme 2) by a procedure invented by Pankiewicz et al. Compound 6 is further converted in a solvent, preferably pyridine, into bicyclic trisanhydride 9 via P1,P4-disubstituted methylenebis-(phosphonic) anhydride 7 and monocyclic intermediate 8 by the action of a carbodiimide. The sequence of reactions can be followed by ³¹P NMR spectroscopy. When formation of 9 is completed, a β-D purine nucleoside 10 is added to the reaction mixture to form P¹,P²,P³,P⁴-tetrasubstituted methylenebis(phosphonic) anhydride 11, which is rather sensitive to moisture, and readily hydrolyzed to the desired product with formula [I] with water.

The β-L-counterpart of [I] can be synthesized by using, instead of 10, the corresponding β-L-nucleoside.

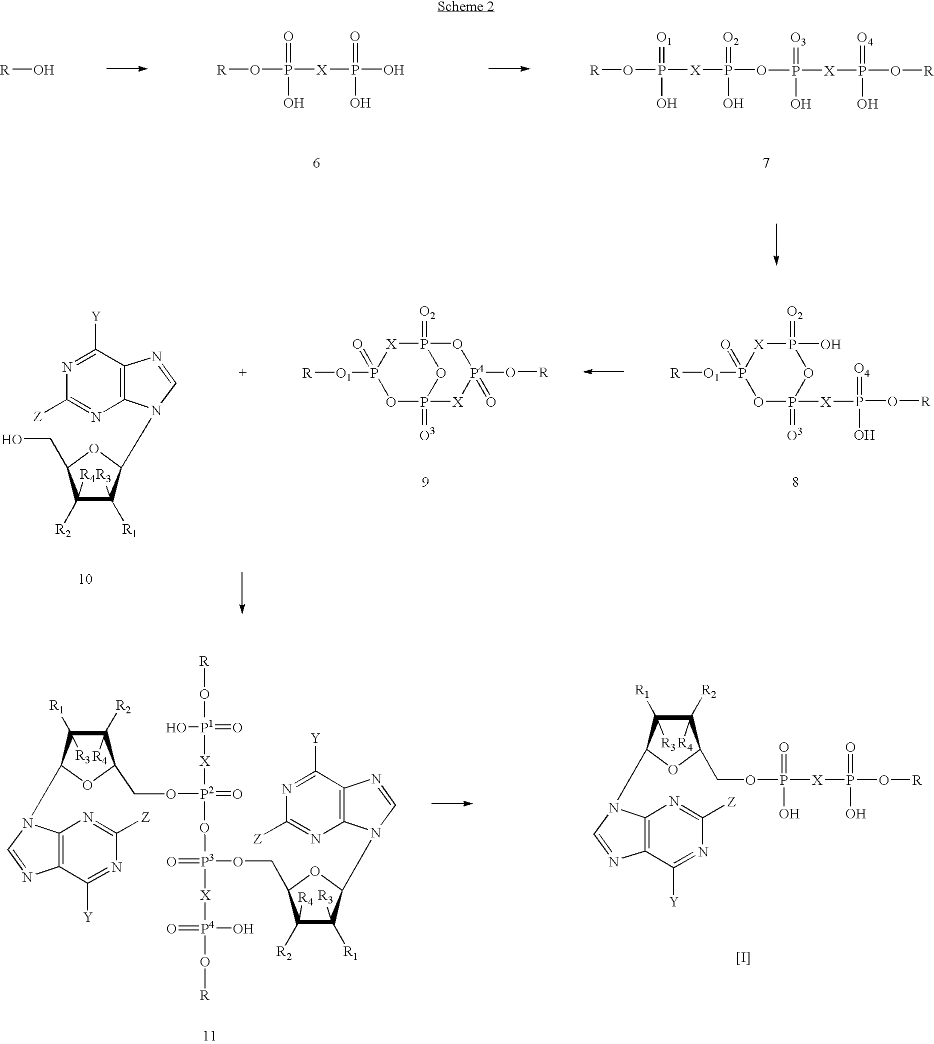

Synthesis of Compounds of Formula [II] and Their Derivatives:

For the preparation of a synthon of R—OH wherein R is formula [II], one of the two methods we invented earlier (Kabat et al., J. Med. Chem., 1987, 30, 924-927; Pankiewicz et al., J. Org. Chem., 1988, 53, 3473-3479) or their modified version (Matulic-Adamic, et al., Synthesis, 1997, 38, 203-206) is exploited.

The preferred starting materials for all the processes for the synthesis of a compound of general formula [II] can be subsumed under general formula IIa as follows:

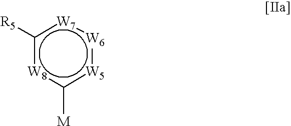

wherein W₅-W₈ are same or different, and independently methyne (—CH═) or azomethyne (—N═);

R⁵ is a halogen (fluorine, chlorine, bromine or iodine), CN, CONH₂, CO₂Me, CO₂Et, or CO₂H; and

M is alkali or alkali earth metal such as lithium, sodium, potassium, magnesium or cadmium.

The starting material of formula IIa are allowed to react with 2,3,4,5-tetra-O-protected aldehydo-D-ribose such as 2,4;3,5-di-O-benzylidene-aldehydo-D-ribose (12, Scheme 3). The reaction is carried out in an appropriate solvent such as ethyl ether or tetrahydrofuran or a mixture of these solvents at a temperature range from −90° C. to 60° C. in a period of from 1 hour to 5 days. The molar ratio of the reactants, IIa to aldehydo-D-ribose 12 can be from 1:1 to 1:10, preferably 1:4. Upon completion of the reaction, water is added to decompose excess metal-complex IIa. Condensation product is obtained from the organic layer by removal of the solvent and chromatographic purification of the residue. The product is a mixture of altro isomer (13) and allo isomer (14). The hydroxyl group of the condensation product 13 or 14 is converted into a leaving group by sulfonylation with a common sulfonylating agent, such as mesyl chloride, tosyl chloride, nisyl chloride, triflyl chloride, tresyl chloride or triflic acid anhydride in pyridine or in an inert solvent such as a chlorinated hydrocarbon, such as methylene chloride, chloroform, ethylene chloride and the like, in the presence of base such as pyridine, triethylamine, p-di-methylpyridine, DBU, DBN or the like. The temperature range of the reaction is from −78° C. to 60° C., preferably at room temperature in a period of from 1 hour to 5 days. The corresponding product (15 or 16) is treated with a strong organic acid such as methanesulfonic acid, p-toluenesulfonic acid, trifluoromethanesulfonic acid or trifluoroacetic acid in an inert organic solvent such as chlorinated hydrocarbons at a temperature range of from 0° C. to 60° C., preferably at room temperature, in a period of from 5 minutes to 24 hours to give the β-C-nucleoside [II] from the altro isomer 15 and the α-C-nucleoside 17 from the allo isomer 16. 2,3,4,5-Di-O-isopropylidene-aldehydo-D-ribose (18) or 2,3,4,5-tetra-O-benzyl-aldehydo-D-ribose (19) can also be used instead of 12 in the above sequence of reactions. By using 2,4;3,5-tetra-O-protected aldehydo-1-ribose instead of 12, the L-nucleoside counterpart of [II] can be obtained.

The substituent in the aglycon R⁵ can be converted into the carboxamide function at various stage of the reaction sequence. Thus, when R⁵ in 13 or 14 is bromine or iodine, they are lithiated with butyllithium in an inert solvent, such as ethyl ether or tetrahydrofuran, or a mixture of inert solvents, such as a mixture of ethyl ether and hexamethylphosphoric triamide, at a temperature range of from −90° C. to 60° C., preferably from −78° C. to 25° C. in a period of from 5 minutes to 5 hours. The lithiated product is then treated with carbon dioxide to give carboxylic acid (13 or 14, R⁵═COOH), which is esterified by treatment with diazomethane in ether to afford methyl ester (13 or 14, R⁵═COOCH₃). Conversion of this ester into carboxamide (13 or 14, R⁵═CONH₂) is performed by treatment of the ester with alcoholic ammonia at a temperature range from 0° C. to 100° C., preferably at 25° C., in a period of from 1 hour to 5 days. This halogen to carboxamide conversion can also be performed of molecule [II] and 17.

When R⁵ of 13 or 14 is carbonitrile (CN), hydration to carboxamide is achieved in aqueous alcohol at reflux temperature with base such as sodium hydroxide, potassium hydroxide, lithium hydroxide and the like or strongly basic ion-exchange resin such as Dowex-1 (OH⁻) or Amberlite 400 (OH⁻).

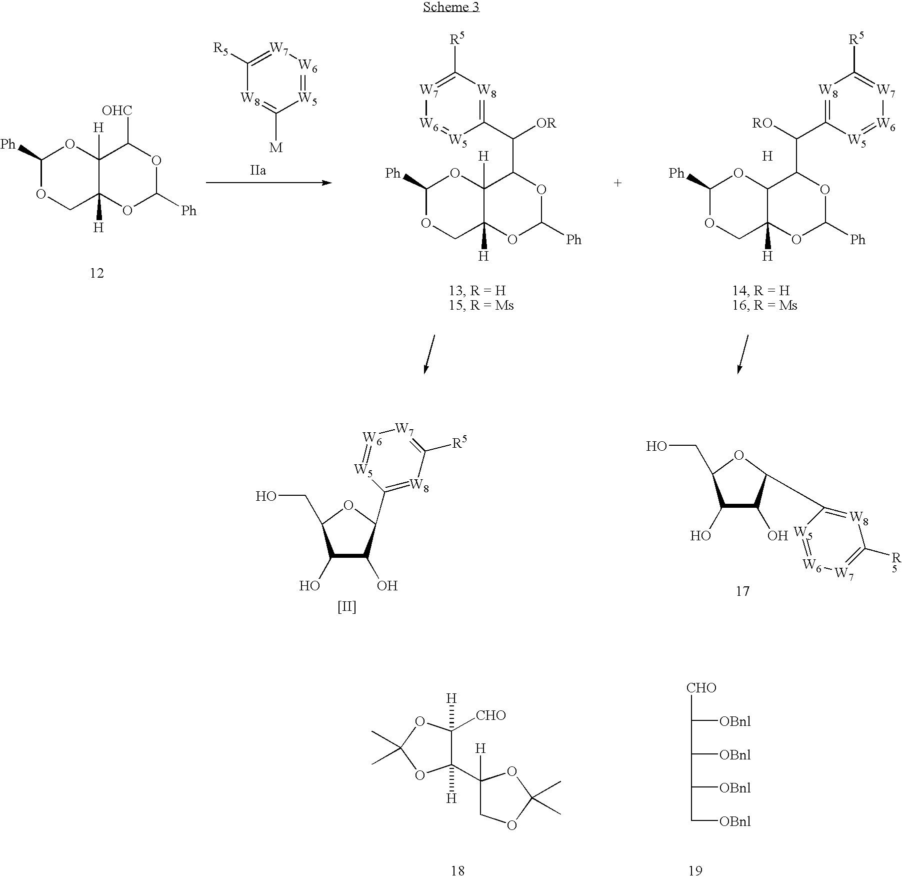

The disadvantage of the above procedure is to prepare starting aldehydo-sugar derivatives 12, 18 or 19, which requires large amounts of toxic alkylmercaptan, mercuric chloride and mercuric oxide. An alternative procedure the present authors developed circumvents such problems.

The starting material for the aglycon is again IIa and the glycon is a protected ribo-γ-lactone, e.g., 5-O-t-butyldimethylsilyl-2,3-O-isopropylidene-D-ribonolactone (20, Scheme 4). These reactants are allowed to react in an inert solvent, such as ethyl ether or tetrahydrofuran or a mixture of these solvents at a temperature range from −90° C. to 60° C., preferably at −78° C., in a period of from 1 hour to 5 days. The molar ratio of the reactants, IIa to lactone 20 can be from 0.1:1 to 1:10, preferably 1:1. Upon completion of the reaction, water is added to decompose excess metal-complex IIa. Condensation product is obtained from the organic layer by removal of the solvent and chromatographic purification of the residue. The product is usually the β-anomer 21. In some cases, hydroxyl group of 21 can be directly removed by reduction with triethylsilane in an inert solvent to give an anomeric mixture of [II] and 17. The ratio of anomers formed is depending upon reduction conditions and not quite predictable. The L-nucleoside counterpart of [II] and 17 can be obtained starting from the L-ribonolactone instead of 20.

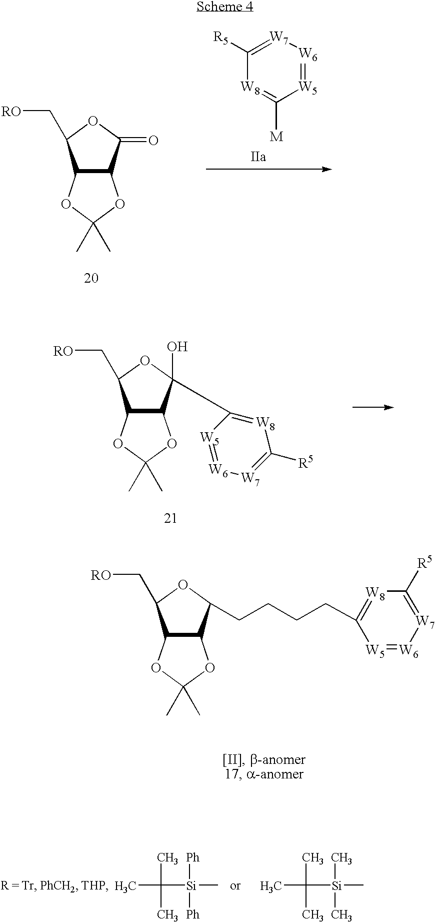

In many cases, however, reduction by number of known procedures does not take place. In such cases, 21 is reduced with sodium borohydride or lithium aluminum hydride, preferably sodium borohydride, to a mixture of open-chain altro and allo isomers 22 and 23 (Scheme 5).

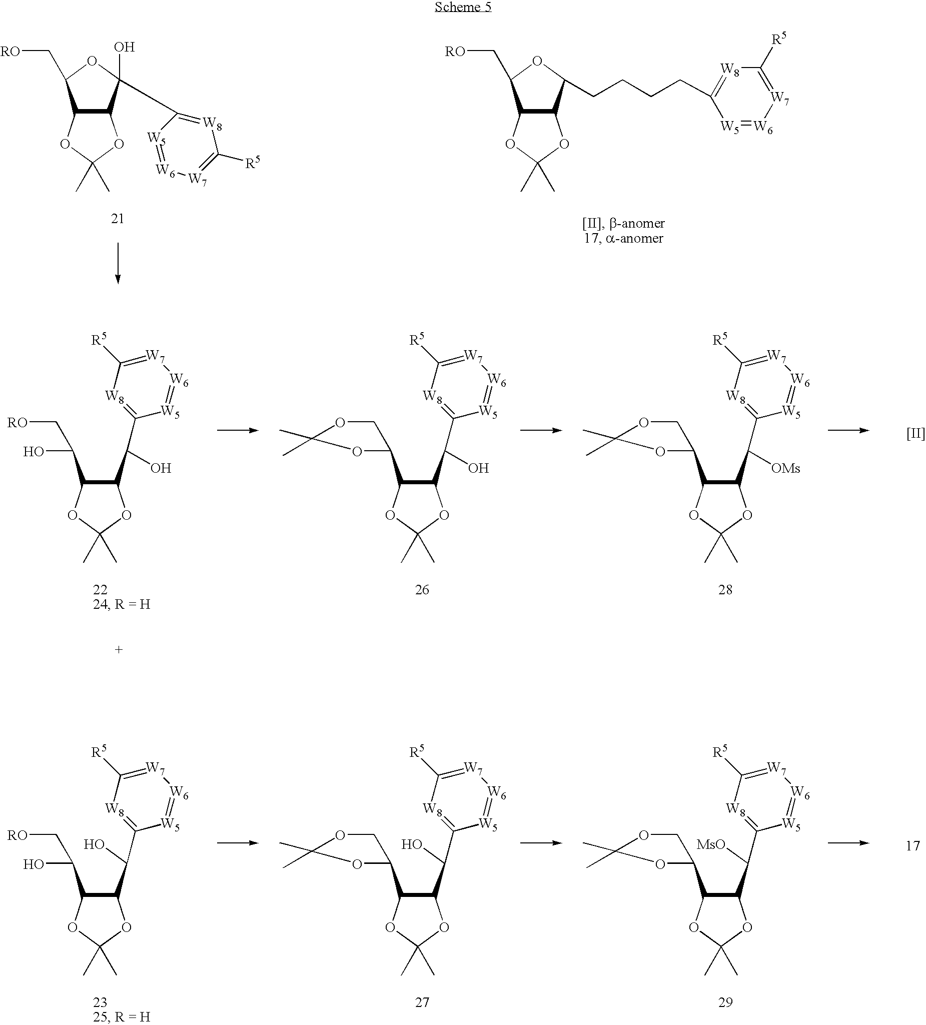

After selective removal of R in 22 and 23 to 24 and 25, respectively, the vicinal diol is protected with isopropylidene, benzylidene, cyclic carbonate, or orthoester group, preferably isopropylidene group to give 26 and 27, respectively. The anomeric hydroxyl group in these compounds can be sulfonylated, preferably mesylated, as described previously to the corresponding products 28 and 29. Upon treatment of the altro isomer 28 with trifluoroacetic acid, the desired β-C-nucleoside [II] is obtained. The allo isomer gives the α-C-nucleoside 17.

Recently, Matulic-Adamic et al. reported that condensation of 2-fluoro-3-lithiopyridine with 20 (R=t-butyldiphenylsilyl) and obtained a single condensation product analogous to 21. They, however, could not remove the anomeric hydroxyl group by reduction with triethylsilane, or any other reducing agents. They treated the condensation product with sodium borohydride in methanol and obtained a mixture of open-chain compounds analogous to a mixture of 23 and 25. They found that application of Mitsunobu reaction directly to the mixture afforded an anomeric mixture of cyclized compounds. Taking advantage of Matulic-Adamic discovery we treat a mixture of 27 and 28 directly with triphenylphosphine and diethyl azodicarboxylate (Mitsunobu reaction) in an inert solvent such as ethyl ether or tetrahydrofuran to give [II] and 17 with various anomeric ratios depending upon reaction conditions.

Synthesis of Compounds of Formula [III]:

When W₂ in compounds of formula [III] is nitrogen, synthesis of [III] is performed by condensation of the base IIIa (Scheme 6) with a D-ribosyl derivative 30 in a chlorinated hydrocarbon (methylene chloride or ethylene chloride) or acetonitrile in the presence of Lewis acid (such as tin chloride, titanium chloride or trimethylsilyl triflate), followed by deprotection. The molar ratio of the reactants, IIIa to sugar 30 can be from 1:0.5 to 1:2, preferably 1:1. The temperature for the reaction can be from −10° C. to 100° C., preferably from 25° C. to 60° C. for a period from 1 hour to 1 week. Upon completion of the reaction, methanol is added to hydrolyze the silyl groups of the product to give 31. The L-nucleoside counterpart of [III] can be obtained when 30 is replaced by the corresponding L-ribosyl derivative.

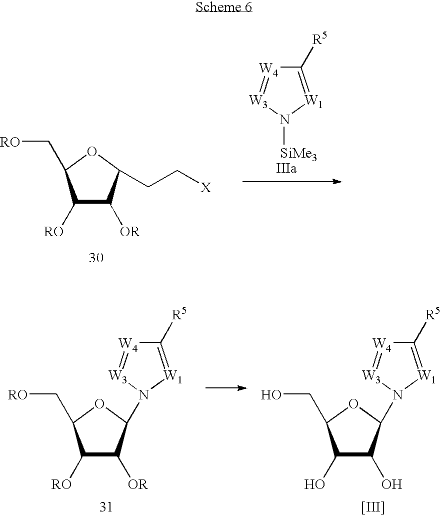

R═Ac, Bz, Bnl; R⁵=halogen, CO₂Me, CN; X═OCH₃, OAc, Cl, Br

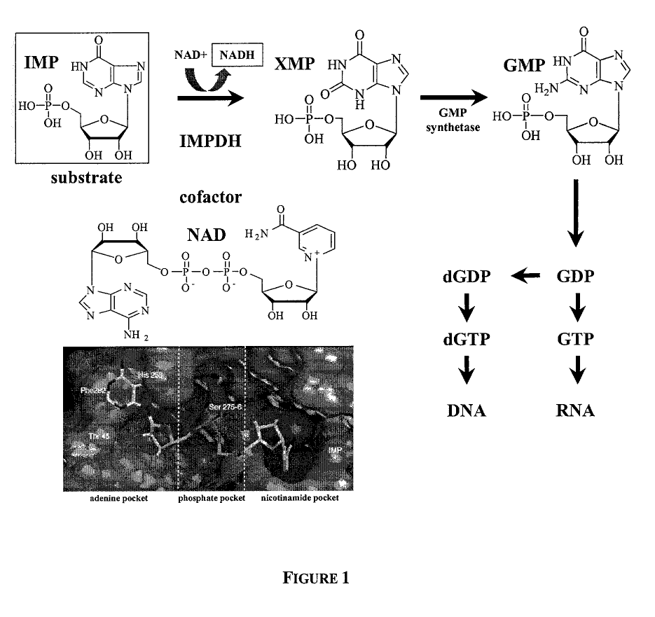

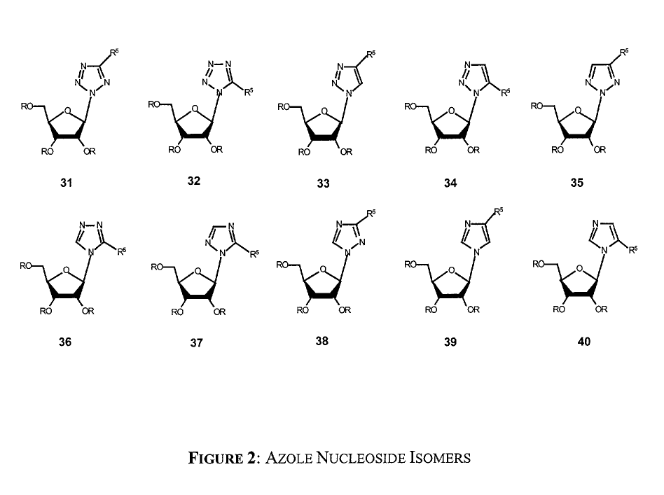

It should be noted that IIIa is a tetrazole (W₁-W₄═N; 31 and 32), triazole (W₃ or W₄═CH; 33 to 38), imidazole (W₁ and W₃═CH; 39 to 40) or pyrazole (W₃ and W₄═CH), condensation reaction affords a mixture of isomers (FIG. 1). R⁵ in [III] is readily modified to carboxamide.

When W₂ in [III] is methyne (—CH═) and W₁, W₃ or W₄ is (N or CH), metalated aglycon IIIb (Scheme 7) is prepared and condensed with D-ribo-γ-lactone derivative (20) according to the procedure described already with Scheme 4 to give 41, from which the anomeric hydroxyl group is removed to afford 42 by one of the procedures already discussed. The metalated aglycons IIIb can be prepared from the readily available cyclic amide or cyclic urea (43) by treatment with phosphorus oxyhalide (X═Cl or Br) to give the corresponding halogeno derivative (44-46), which is then treated with n- or sec- or t-butyllithium in tetrahydrofuran.

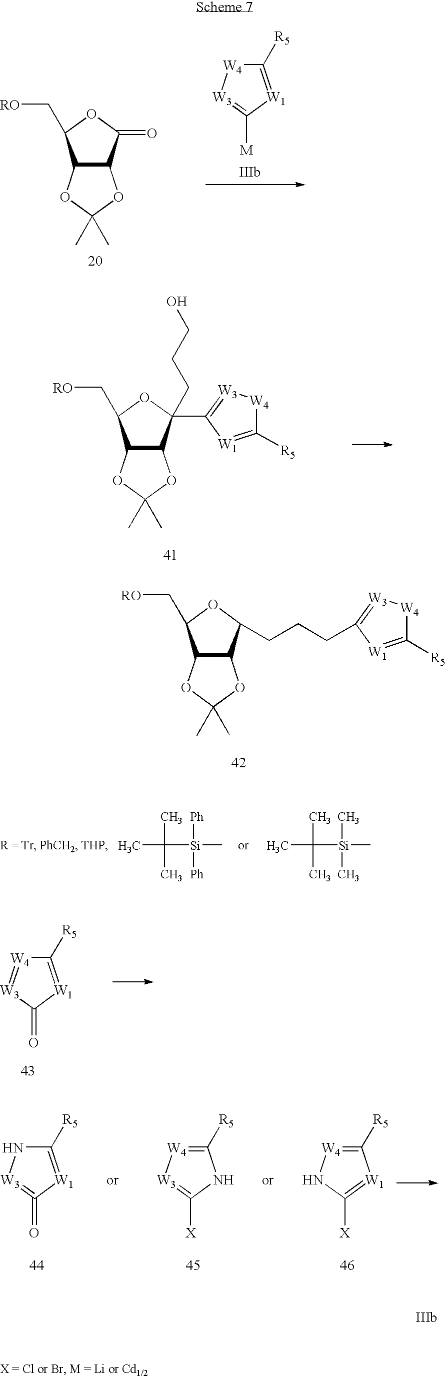

Compounds of formula III can also be synthesized from a ribosyl nitrile or α-ribosyl acetonitrile. 2,3-O-Isopropylidene-5-O-(t-butyldiphenylsilyl)-β-D-ribosylnitrile (49, Scheme 8) is prepared from the known β-D-ribofuranosylnitrile (47) by isopropylidenation by treatment with 2,2-dimethoxypropane in acetone in the presence of catalytic amount of acid, such as sulfuric, p-toluenesulfonic, methanesulfonic or trifluoromethanesulfonic acid, followed by treatment of the product 48 with t-butyldiphenylsilyl chloride in pyridine in the presence of p-dimethylaminopyridine. Instead of using 2,2-dimethoxypropane, when α,α-dimethoxytoluene is used in an inert solvent, 2,3-O-benzylidene derivative is formed. Treatment of 47 with cyclohexanone and acid, 2,3-cyclohexylidene derivative is formed instead of 48. These base stable 2,3-di-O-protected intermediates can also be used for the present invention. The 5-position of 48 or an analogue can also be protected with tetrahydropyranyl, benzoyl, t-butyldimethylsilyl or benzyl group. Compound 49 is reduced with sodium borohydride or lithium aluminum hydride or a like in an inert solvent, preferably, tetrahydrofuran, to give the aminomethyl product 50, which is converted into the diazomethane 51 by diazotization. 1,3-Di-polar addition of 51 with methyl propiolate affords the pyrazole derivative 52. Ammonolysis of 52 gives carboxamide derivative 53, which, upon treatment with fluoride ion (tetrabutylammonium fluoride or triethylammonium hydrogen fluoride) affords the 2,3-O-iso-propyliene derivative 54, in which only the 5′-hydroxyl group is free. Starting from the β-L-ribofuranosylnitrile in place of 47, the β-L C-nucleoside counterpart of 54 can be prepared. Compound 54 or its β-L counterpart can be used directly in the synthesis of the NAD analogues discussed with Scheme 1.

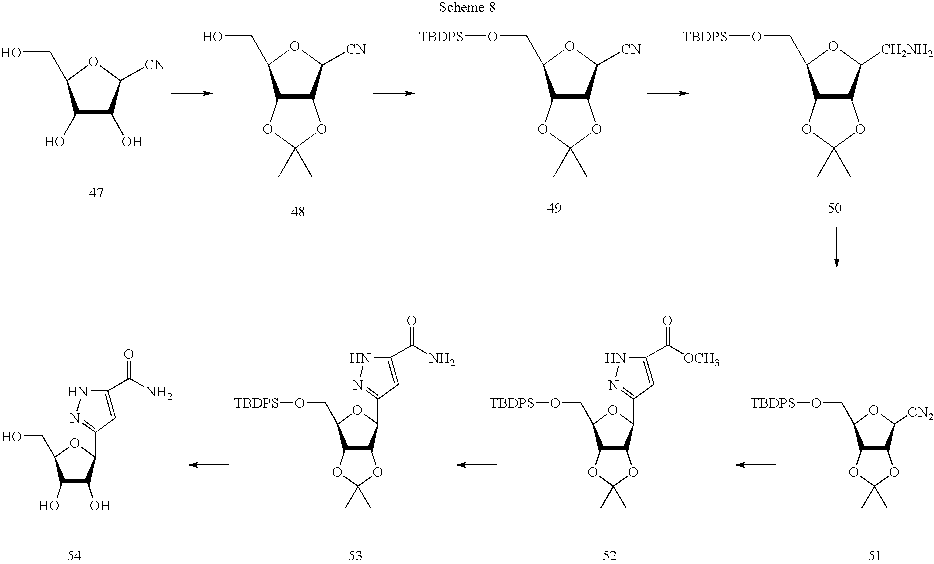

Compound 49 is a versatile intermediate. It can be converted into the thioamide 55 (Scheme 9) which is cyclized with alkyl (methyl or ethyl) bromopyruvate in pyridine gives the thiazole C-nucleoside 56. Ammonolysis of the ester 56, followed by fluoride treatment affords the 2′,3′-di-O-protected C-nucleoside 57, which can be used directly in the NAD analogue synthesis as discussed with Scheme 1.

In a similar manner, 49 can be converted into ribosylamide or selenamide (the sulfur in 55 is displaced by O or Se), from which oxazole and selenazole analogues 58 and 59, respectively, can be prepared.

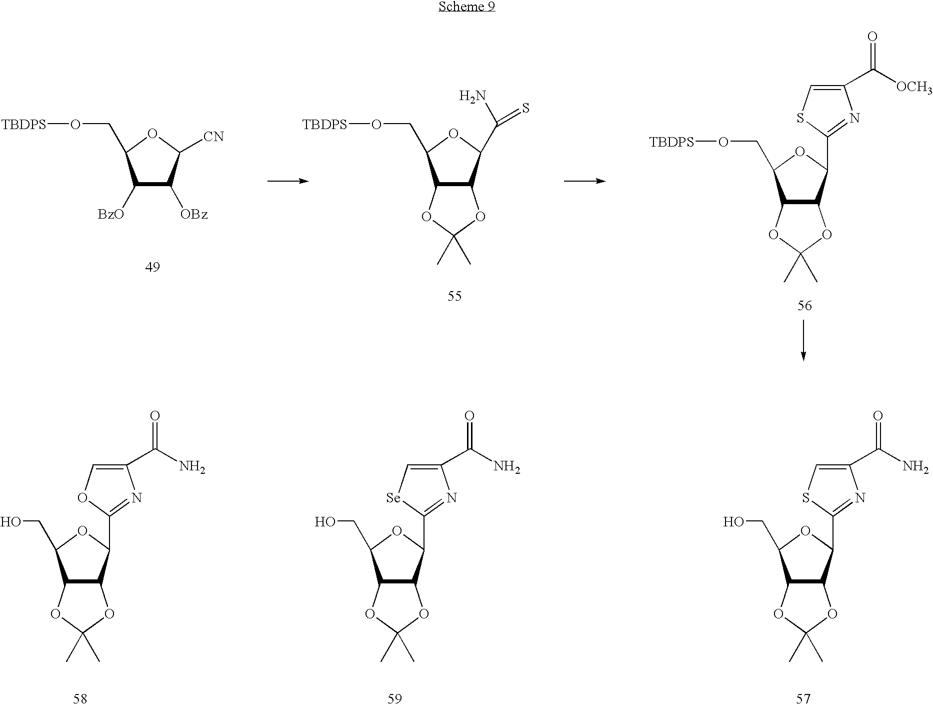

Synthesis of Compounds of Formulae [IV] and [V]:

These compounds are synthesized by the procedure reported by Jones and Mills (J. Med. Chem., 1971,14, 305).

The following examples are illustrative of the processes and products of the present invention, but are not intended to, and should not be interpreted to, limit in any way the invention set forth in the claims that follow thereafter.

### EXAMPLES

Anhydrous solvents were purchased from Aldrich Chemical Company, Inc. (Milwaukee). Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed in order to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were recorded on a JEOL JMS-SX/SX102A/E mass spectrometer. Elemental analyses were performed by Atlantic Microlab Inc. (Norcross, Ga.). Analytic TLC was performed on Whatman LK6F silica gel plates, and preparative TLC on Whatman PK5F silica gel plates. Column chromatography was carried out on Silica Gel (Fisher, S733-1) at atmospheric pressure.

### Example 1

### 5-(2,4;3,5-Di-O-benzylidene-D-hexityl)-3-bromopyridine (13 and 14, R═H, R⁵═Br, W₆═N, W₅═W₇═W₈═CH)

To a solution of 3,5-dibromopyridine (1.45 g, 6.12 mmol) in dry ethyl ether (50 mL) is slowly added (ca. 10 min) a solution of n-butyllithium (2.35 mL, 2.6 M solution in n-hexane, 6.12 mmol) below −50° C. under argon atmosphere. After addition is completed, the reaction mixture is further stirred for 15 minutes. The mixture is then cooled to −78° C. and a solution of 2,4;3,5-di-O-benzylidene-D-aldehydo-ribose (12, 500 mg, 1.53 mmol) in tetrahydrofuran (5 mL) is added dropwise, and then the reaction mixture is allowed to warm to room temperature. Water (50 mL) is added to the reaction mixture. The organic layer is separated, washed with brine (30 mL×3), dried over sodium sulfate, and then concentrated in vacuo. The residue is chromatographed on a column of silica gel (20 g) using first methylene chloride and then 10% ethyl ether in methylene chloride as the eluent, to give a slightly yellow product, which is crystallized from ethanol (420 mg, 52%), mp. 170-174° C. ¹H NMR (CDCl₃): δ 3.8-4.4 (5H, m, H-2′,3′,4′,5′,5″), 5.0 (1H, M, H-1′), 5.60, 5.64, 5.71, 5.74 (2H, 4S, benzylidene-CH<), 7.38 (10H, s, Ph), 7.98 (1H, m, H-2), 8.58 (2H, m, H-4,5).

This solid is a mixture of 13 and 14.

By following the same procedure but using the corresponding aldehydo-D-ribose (19 and 20) as the starting materials, 5-(2,3;4,5-di-O-isopropylidene-D-hexityl)-3-bromopyrimidine and 5-(2,3,4,5-tetra-O-benzyl-D-hexityl)-3-bromopyridine are also prepared as altro/allo mixtures.

By following the same procedure but using the corresponding bases as the starting materials, the following compounds are also prepared as altro/allo mixtures:

4-(2,4;3,5-Di-O-benzylidene-D-hexityl)-2-bromopyridine,

3-(2,4;3,5-Di-O-benzylidene-D-hexityl)-2-bromopyridine,

5-(2,4;3,5-Di-O-benzylidene-D-hexityl)-2-bromopyridine,

6-(2,4;3,5-Di-O-benzylidene-D-hexityl)-2-bromopyridine,

6-(2,4;3,5-Di-O-benzylidene-D-hexityl)-3-bromopyridine,

4-(2,4;3,5-Di-O-benzylidene-D-hexityl)-3-bromopyridine,

2-(2,4;3,5-Di-O-benzylidene-D-hexityl)-3-bromopyridine,

2-(2,4;3,5-Di-O-benzylidene-D-hexityl)-4-bromopyridine,

3-(2,4;3,5-Di-O-benzylidene-D-hexityl)-4-bromopyridine,

4-(2,4;3,5-Di-O-benzylidene-D-hexityl)-2-chloropyridine,

3-(2,4;3,5-Di-O-benzylidene-D-hexityl)-2-chloropyridine,

5-(2,4;3,5-Di-O-benzylidene-D-hexityl)-2-chloropyridine,

6-(2,4;3,5-Di-O-benzylidene-D-hexityl)-2-chloropyridine,

6-(2,4;3,5-Di-O-benzylidene-D-hexityl)-3-chloropyridine,

5-(2,4;3,5-Di-O-benzylidene-D-hexityl)-3-chloropyridine,

4-(2,4;3,5-Di-O-benzylidene-D-hexityl)-3-chloropyridine,

2-(2,4;3,5-Di-O-benzylidene-D-hexityl)-3-chloropyridine,

2-(2,4;3,5-Di-O-benzylidene-D-hexityl)-4-chloropyridine,

3-(2,4;3,5-Di-O-benzylidene-D-hexityl)-4-chloropyridine,

4-(2,4;3,5-Di-O-benzylidene-D-hexityl)-2-iodopyridine,

3-(2,4;3,5-Di-O-benzylidene-D-hexityl)-2-iodopyridine,

5-(2,4;3,5-Di-O-benzylidene-D-hexityl)-2-iodopyridine,

6-(2,4;3,5-Di-O-benzylidene-D-hexityl)-2-iodopyridine,

6-(2,4;3,5-Di-O-benzylidene-D-hexityl)-3-iodopyridine,

5-(2,4;3,5-Di-O-benzylidene-D-hexityl)-3-iodopyridine,

4-(2,4;3,5-Di-O-benzylidene-D-hexityl)-3-iodopyridine,

2-(2,4;3,5-Di-O-benzylidene-D-hexityl)-3-iodopyridine,

2-(2,4;3,5-Di-O-benzylidene-D-hexityl)-4-iodopyridine,

3-(2,4;3,5-Di-O-benzylidene-D-hexityl)-4-iodopyridine,

4-(2,4;3,5-Di-O-benzylidene-D-hexityl)-2-cyanopyridine,

3-(2,4;3,5-Di-O-benzylidene-D-hexityl)-2-cyanopyridine,

5-(2,4;3,5-Di-O-benzylidene-D-hexityl)-2-cyanopyridine,

6-(2,4;3,5-Di-O-benzylidene-D-hexityl)-2-cyanopyridine,

5-(2,4;3,5-Di-O-benzylidene-D-hexityl)-3-cyanopyridine,

6-(2,4;3,5-Di-O-benzylidene-D-hexityl)-3-cyanopyridine,

4-(2,4;3,5-Di-O-benzylidene-D-hexityl)-3-cyanopyridine,

2-(2,4;3,5-Di-O-benzylidene-D-hexityl)-3-cyanopyridine,

2-(2,4;3,5-Di-O-benzylidene-D-hexityl)-4-cyanopyridine, and

3-(2,4;3,5-Di-O-benzylidene-D-hexityl)-4-cyanopyridine.

By following the same procedure but using 2,4;3,5-di-O-benzylidene-L-aldehydo-ribose, thye L-counterpart of 13 and 14 are prepared.

### Example 2

### Methyl 5-(2,4;3,5-di-O-benzylidene-D-hexityl)nicotinate (13 and 14, R═H,R⁵═CO₂CH₃, W₆═N, W₅═W₇═W₈═CH)

To a solution of 5-(2,4;3,5-di-O-benzylidene-D-hexityl)-3-bromopyridine (200 mg, 0.43 mmol) in a mixture of hexamethylphosphoric triamide (0.5 mL) and ethyl ether (5 mL) is added a solution of butyllithium (2 mL of 2.5 M solution in n-hexane, 5 mmol) under argon atmosphere at −78° C. After the addition, the mixture is stirred at −78° C. for 15 minutes. A large excess of solid carbon dioxide is added, and the mixture is allowed to warm to room temperature. The mixture is acidified by addition of 1N hydrochloric acid to pH 4, and the organic layer is washed with brine (3×5 mL), dried over sodium sulfate. After removal of sodium sulfate by filtration, the filtrate is cooled to 0° C. and treated with a large excess of ethereal diazomethane. Excess diazomethane is then destroyed by addition of acetic acid. The mixture is concentrated in vacuo, and the residue is chromatographed on a silica gel column using n-hexane-ethyl acetate (7:3) as the eluent. Methyl 5-(2,4;3,5-di-O-benzylidene-D-hexityl)nicotinate is obtained as a mixture of altro/allo epimers (13 and 14, R═H, R⁵═CO₂CH₃, W₆═N,W₅═W₇═W₈═CH), 121 mg (65%), mp 175-178° C. ¹H NMR (CDCl₃): δ 3.5-4.4 (5H, m, H-2′,3′,4′,5′,5″), 3.92, 3.94 (3H, 2s, Me ester), 5.11 (1H, m, H-1′), 5.58, 5.65, 5.71, 5.75 (2H, 4s, benzylidene-CH<), 7.37, 7.38 (10H, 2s, Ph),8.42, 8.83, 9.13 (3H, 3s, pyridine).

By following the same procedure but using the corresponding pyridine intermediates, the following methyl esters are prepared:

Methyl 6-(2,4;3,5-Di-O-benzylidene-D-hexityl)nicotinate,

Methyl 4-(2,4;3,5-Di-O-benzylidene-D-hexityl)nicotinate,

Methyl 2-(2,4;3,5-Di-O-benzylidene-D-hexityl)nicotinate,

Methyl 4-(2,4;3,5-Di-O-benzylidene-D-hexityl)picolinate,

Methyl 3-(2,4;3,5-Di-O-benzylidene-D-hexityl)picolinate,

Methyl 5-(2,4;3,5-Di-O-benzylidene-D-hexityl)picolinate,

Methyl 6-(2,4;3,5-Di-O-benzylidene-D-hexityl)picolinate,

Methyl 2-(2,4;3,5-Di-O-benzylidene-D-hexityl)isonicotinate, and

Methyl 3-(2,4;3,5-di-O-benzylidene-D-hexityl)isonicotinate,

By following the same procedure but using the corresponding L-intermediates, the L-counterpart of the above compounds are synthesized.

### Example 3

### Methyl 5-(2,4;3,5-di-O-benzylidene-D-hexityl)nicotinamide (13 and 14, R═H, R⁵═CONH₂, W₆═N, W₅═W₇═W₈═CH)

An altro/allo epimeric mixture of methyl 5-(2,4;3,5-di-O-benzylidene-D-hexityl)-nicotinate (220 mg, 0.48 mmol) is treated with saturated methanolic ammonia containing a catalytic amount of sodium hydride (ca. 2 mg), and the mixture is stirred at room temperature overnight. The solvent is removed in vacuo, and the residue is chromatographed on a silica gel column using chloroform-methanol (95:5) as the eluent. Methyl 5-(2,4;3,5-di-O-benzylidene-D-hexityl)nicotinamide is obtained as a mixture of altro and allo epimers (13 and 14, R═H, R⁵═CONH₂, W₆═N,W₅═W₇═W₈═CH), as a form, 185 mg (87%). ¹H NMR (Me₂SO-d₆): δ 3.5-4.3 (5H, m, H-2′,3′,4′,5′,5″), 5.00 (1H, m, H-1′), 5.64 5.76, 5.77, 5.89 (2H, 4s, benzylidene-CH<), 7.39 (10H, s, phenyl), 7.50, 8.21 (4H, 2 broad s, CONH₂), 8.29, 8.68, 8.95 (3H, 3m, pyridine).

By following the same procedure but using the corresponding methyl esters, the following carboxamides are prepared:

6-(2,4;3,5-Di-O-benzylidene-D-hexityl)nicotinamide,

4-(2,4;3,5-Di-O-benzylidene-D-hexityl)nicotinamide,

2-(2,4;3,5-Di-O-benzylidene-D-hexityl)nicotinamide,

4-(2,4;3,5-Di-O-benzylidene-D-hexityl)picolinamide,

3-(2,4;3,5-Di-O-benzylidene-D-hexityl)picolinamide,

5-(2,4;3,5-Di-O-benzylidene-D-hexityl)picolinamide,

6-(2,4;3,5-Di-O-benzylidene-D-hexityl)picolinamide,

2-(2,4;3,5-di-O-benzylidene-D-hexityl)isonicotinamide, and

3-(2,4;3,5-di-O-benzylidene-D-hexityl)isonicotinamide.

The L-isomers are also prepared in a similar manner by starting from the corresponding L-intermediates.

### Example 4

### 5-(2,4;3,5-Di-O-benzylidene-1-O-mesyl-D-hexityl)-3-bromopyridine (13 and 14, R=Ms, R⁵═Br, W₆═N, W₅═W₇═W₈═CH)

An altro/allo mixture of 5-(2,4;3,5-di-O-benzylidene-D-hexityl)-3-bromopyridine (226 mg, 0.47 mmol) is dissolved in pyridine (10 μL) and treated at 0° C. with mesyl chloride (72 μL, 0.93 mmol). The mixture is stirred at room temperature for 20 hours, and then concentrated in vacuo. The residue is dissolved in ethyl acetate (20 mL) and the solution is washed successively with equal volumes of water, 1N hydrochloric acid, brine, saturated sodium bicarbonate solution, and dried over sodium sulfate. After condensation of the solution to dryness, the residue is chromatographed on a silica gel column using n-hexane-ethyl acetate (3:7) as the eluent. 5-(2,4,3,5-Di-O-benzylidene-1-O-mesyl-D-hexityl)-3-bromopyridine as an epimeric altro/allo mixture is obtained as microcrystals, 223 mg (91%), mp 125-134° C. ¹H NMR (CDCl3):δ 2.93, 2.95 (3H, 2s, Ms), 3.7-4.5 (5H, m, H-2′,3′,4′,5′,5″), 5.51, 5.66, 5.79 (2H, 3s, benzylidene-CH<), 5.90 (1H, m, H-1′), 7.40 (10H, s, phenyl), 8.0 (1H, m, H-2), 8.7 (2H, m, H-4,6).

By following the same procedure but using the corresponding altro/allo mixture of (2,4;3,5-di-O-benzylidene-D-hexityl)pyridines as the starting materials, the following mesylates are also prepared:

4-(2,4;3,5-Di-O-benzylidene-1-O-mesyl-D-hexityl)-2-bromopyridine,

3-(2,4;3,5-Di-O-benzylidene-1-O-mesyl-D-hexityl)-2-bromopyridine,

5-(2,4;3,5-Di-O-benzylidene-1-O-mesyl-D-hexityl)-2-bromopyridine,

6-(2,4;3,5-Di-O-benzylidene-1-O-mesyl-D-hexityl)-2-bromopyridine,

6-(2,4;3,5-Di-O-benzylidene-1-O-mesyl-D-hexityl)-3-bromopyridine,

4-(2,4;3,5-Di-O-benzylidene-1-O-mesyl-D-hexityl)-3-bromopyridine,

2-(2,4;3,5-Di-O-benzylidene-1-O-mesyl-D-hexityl)-3-bromopyridine,

2-(2,4;3,5-Di-O-benzylidene-1-O-mesyl-D-hexityl)-4-bromopyridine,

3-(2,4;3,5-Di-O-benzylidene-1-O-mesyl-D-hexityl)-4-bromopyridine,

4-(2,4;3,5-Di-O-benzylidene-1-O-mesyl-D-hexityl)-2-chloropyridine,

3-(2,4;3,5-Di-O-benzylidene-1-O-mesyl-D-hexityl)-2-chloropyridine,

5-(2,4;3,5-Di-O-benzylidene-1-O-mesyl-D-hexityl)-2-chloropyridine,

6-(2,4;3,5-Di-O-benzylidene-1-O-mesyl-D-hexityl)-2-chloropyridine,

6-(2,4;3,5-Di-O-benzylidene-1-O-mesyl-D-hexityl)-3-chloropyridine,

5-(2,4;3,5-Di-O-benzylidene-1-O-mesyl-D-hexityl)-3-chloropyridine,

4-(2,4;3,5-Di-O-benzylidene-1-O-mesyl-D-hexityl)-3-chloropyridine,

2-(2,4;3,5-Di-O-benzylidene-1-O-mesyl-D-hexityl)-3-chloropyridine,

2-(2,4;3,5-Di-O-benzylidene-1-O-mesyl-D-hexityl)-4-chloropyridine,

3-(2,4;3,5-Di-O-benzylidene-1-O-mesyl-D-hexityl)-4-chloropyridine,

4-(2,4;3,5-Di-O-benzylidene-1-O-mesyl-D-hexityl)-2-iodopyridine,

3-(2,4;3,5-Di-O-benzylidene-1-O-mesyl-D-hexityl)-2-iodopyridine,

5-(2,4;3,5-Di-O-benzylidene-1-O-mesyl-D-hexityl)-2-iodopyridine,

6-(2,4;3,5-Di-O-benzylidene-1-O-mesyl-D-hexityl)-2-iodopyridine,

6-(2,4;3,5-Di-O-benzylidene-1-O-mesyl-D-hexityl)-3-iodopyridine,

5-(2,4;3,5-Di-O-benzylidene-1-O-mesyl-D-hexityl)-3-iodopyridine,

4-(2,4;3,5-Di-O-benzylidene-1-O-mesyl-D-hexityl)-3-iodopyridine,

2-(2,4;3,5-Di-O-benzylidene-1-O-mesyl-D-hexityl)-3-iodopyridine,

2-(2,4;3,5-Di-O-benzylidene-1-O-mesyl-D-hexityl)-4-iodopyridine,

3-(2,4;3,5-Di-O-benzylidene-1-O-mesyl-D-hexityl)-4-iodopyridine,

4-(2,4;3,5-Di-O-benzylidene-1-O-mesyl-D-hexityl)-2-cyanopyridine,

3-(2,4;3,5-Di-O-benzylidene-1-O-mesyl-D-hexityl)-2-cyanopyridine,

5-(2,4;3,5-Di-O-benzylidene-1-O-mesyl-D-hexityl)-2-cyanopyridine,

6-(2,4;3,5-Di-O-benzylidene-1-O-mesyl-D-hexityl)-2-cyanopyridine,

5-(2,4;3,5-Di-O-benzylidene-1-O-mesyl-D-hexityl)-3-cyanopyridine,

6-(2,4;3,5-Di-O-benzylidene-1-O-mesyl-D-hexityl)-3-cyanopyridine,

4-(2,4;3,5-Di-O-benzylidene-1-O-mesyl-D-hexityl)-3-cyanopyridine,

2-(2,4;3,5-Di-O-benzylidene-1-O-mesyl-D-hexityl)-3-cyanopyridine,

2-(2,4;3,5-Di-O-benzylidene-1-O-mesyl-D-hexityl)-4-cyanopyridine,

3-(2,4;3,5-Di-O-benzylidene-1-O-mesyl-D-hexityl)-4-cyanopyridine,

Methyl 6-(2,4;3,5-di-O-benzylidene-1-O-mesyl-D-hexityl)nicotinate,

Methyl 4-(2,4;3,5-di-O-benzylidene-1-O-mesyl-D-hexityl)nicotinate,

Methyl 2-(2,4;3,5-di-O-benzylidene-1-O-mesyl-D-hexityl)nicotinate,

Methyl 4-(2,4;3,5-di-O-benzylidene-1-O-mesyl-D-hexityl)picolinate,

Methyl 3-(2,4;3,5-di-O-benzylidene-1-O-mesyl-D-hexityl)picolinate,

Methyl 5-(2,4;3,5-di-O-benzylidene-1-O-mesyl-D-hexityl)picolinate,

Methyl 6-(2,4;3,5-di-O-benzylidene-1-O-mesyl-D-hexityl)picolinate,

Methyl 2-(2,4;3,5-di-O-benzylidene-1-O-mesyl-D-hexityl)isonicotinate,

Methyl 3-(2,4;3,5-di-O-benzylidene-1-O-mesyl-D-hexityl)isonicotinate,

2-(2,4;3,5-Di-O-benzylidene-1-O-mesyl-D-hexityl)nicotinamide,

4-(2,4;3,5-Di-O-benzylidene-1-O-mesyl-D-hexityl)picolinamide,

3-(2,4;3,5-Di-O-benzylidene-1-O-mesyl-D-hexityl)picolinamide,

5-(2,4;3,5-Di-O-benzylidene-1-O-mesyl-D-hexityl)picolinamide,

6-(2,4;3,5-Di-O-benzylidene-1-O-mesyl-D-hexityl)picolinamide,

2-(2,4;3,5-Di-O-benzylidene-1-O-mesyl-D-hexityl)isonicotinamide, and

3-(2,4;3,5-Di-O-benzylidene-1-O-mesyl-D-hexityl)isonicotinamide.

By following the same procedure but using tosyl chloride, triflyl chloride, triflyl anhydride or tresyl chloride instead of mesyl chloride, the corresponding sulfonylates of the above mesylates are also prepared.

In a similar manner, the corresponding L-derivatives are prepared.

### Example 5

### 5-(β-D-Ribofuranosyl)nicotinamide (II, R⁵═CONH₂, W₆═N, W₅═W₇═W₈═CH) and 5-(α-D-ribofuranosyl)nicotinamide (17, R⁵═CONH₂, W₆═N, W₅═W₇═W₈═CH)

An altro/allo mixture of 5-(2,4;3,5-di-O-benzylidene-1-O-mesyl-D-hexityl)-nicotinamide (48 mg, 0.19 nM) is dissolved in a mixture of trifluoroacetic acid and chloroform (4:1 v/v), and the mixture is stirred for 15 minutes at room temperature. Water (8 mL) is added and the aqueous layer is washed five times with ethyl ether (equal volume each). After evaporation of the aqueous solution, the residue is chromatographed on a silica gel column using chloroform-methanol (9:1 v/v) as the eluent. 5-β-D-Ribofuranosyl)nicotinamide [II, R⁵═CONH₂, W₆═N, W₅═W₇═W₈═CH) is eluted first from the column, and obtained as a powder. ¹H NMR (Me₂SO-d₆): δ 8.92 (1H, d, H-4, spacing 1.92 Hz), 8.70 (1H, d, H-6, spacing 1.92 Hz), 8.16 (1H, m, H-2), 4.68 (1H, d, H-1′, J_{1′,2′}=7.4 Hz), 3.06-3.16 (5H, m, H-2′,3′,4′,5′,5″). 5-(α-D-Ribofuranosyl)nicotinamide [17, R⁵═CONH₂, W₆═N, W₅═W₇═W₈═CH) is eluted next from the column, and crystallized from methanol, mp 210-212° C. ¹H NMR (Me₂SO-d₆): δ 8.89 (1H, bs, H-2), 8.59 (1H, bs, H-6), 8.14 (1H, bs, H-4), 5.08 (1H, bs, H-1′), 3.17-4.12 (5H, m, H-2′,3′,4′,5′,5″).

By following the same procedure but using the corresponding altro/allo mixture of (2,4;3,5-di-O-benzylidene-D-hexityl)pyridines as the starting materials, the following pyridine C-nucleosides are also prepared:

3-(β-D-ribofuranosyl)-2-bromopyridine, 4-(β-D-ribofuranosyl)-2-bromopyridine, 5-(β-D-ribofuranosyl)-2-bromopyridine, 6-(β-D-ribofuranosyl)-2-bromopyridine, 6-(β-D-ribo-furanosyl)-3-bromopyridine, 4-(β-D-ribofuranosyl)-3-bromopyridine, 2-(β-D-ribo-furanosyl)-3-bromopyridine, 2-(β-D-ribofuranosyl)-4-bromopyridine, 3-(β-D-ribo-furanosyl)-4-bromopyridine, 4-(β-D-ribofuranosyl)-2-chloropyridine, 3-(β-D-ribo-furanosyl)-2-chloropyridine, 5-(β-D-ribo-furanosyl)-2-chloropyridine, 6-(β-D-ribo-furanosyl)-2-chloropyridine, 6-(β-D-ribofuranosyl)-3-chloropyridine, 5-(β-D-ribofuranosyl)-3-chloropyridine, 4-(β-D-ribofuranosyl)-3-chloro-pyridine, 2-(β-D-ribofuranosyl)-3-chloro-pyridine, 2-(β-D-ribofuranosyl)-4-chloropyridine, 3-(β-D-ribofuranosyl)-4-chloropyridine, 4-(β-D-ribofuranosyl)-2-iodopyridine, 3-(β-D-ribo-furanosyl)-2-iodopyridine, 5-(β-D-ribo-furanosyl)-2-iodopyridine, 6-(β-D-ribofuranosyl)-2-iodopyridine, 6-(β-D-ribofuranosyl)-3-iodopyridine, 5-(β-D-ribofuranosyl)-3-iodopyridine, 4-(β-D-ribofuranosyl)-3-iodopyridine, 2-(β-D-ribofuranosyl)-3-iodopyridine, 2-(β-D-ribo-furanosyl)-4-iodopyridine, 3-(β-D-ribo-furanosyl)-4-iodopyridine, 4-(β-D-ribofuranosyl)-2-cyanopyridine, 3-(β-D-ribofuranosyl)-2-cyanopyridine, 5-(β-D-ribofuranosyl)-2-cyano-pyridine, 6-(β-D-ribofuranosyl)-2-cyano-pyridine, 5-(β-D-ribofuranosyl)-3-cyanopyridine, 6-(β-D-ribofuranosyl)-3-cyanopyridine, 4-(β-D-ribofuranosyl)-3-cyanopyridine, 2-(β-D-ribo-furanosyl)-3-cyanopyridine, 2-(β-D-ribo-furanosyl)-4-cyanopyridine, 3-(β-D-ribofuranosyl)-4-cyanopyridine, methyl 6-(D-D-ribo-furanosyl)nicotinate, methyl 4-(β-D-ribofuranosyl)nicotinate, methyl 2-(β-D-ribo-furanosyl)nicotinate, methyl 4-(β-D-ribofuranosyl)picolinate, methyl 3-(β-D-ribo-furanosyl)picolinate, methyl 5-(β-D-ribofuranosyl)picolinate, methyl 6-(β-D-ribo-furanosyl)picolinate, methyl 2-(β-D-ribofuranosyl)isonicotinate, methyl 3-(β-D-ribo-furanosyl)-isoncotinate, methyl 6-(β-D-ribofuranosyl)nicotinate, methyl 4-(β-D-ribo-furanosyl)nicotinate, 2-(β-D-ribofuranosyl)nicotinamide, 4-(β-D-ribofuranosyl)-picolinamide, 3-(β-D-ribo-furanosyl)picolinamide, 5-(β-D-ribofuranosyl)picolinamide, 6-(β-D-ribofuranosyl)-picolinamide, 2-(β-D-ribofuranosyl)isonicotinamide, 3-(β-D-ribo-furanosyl)isonicotinamide, 4-(α-D-ribofuranosyl)-2-bromopyridine, 3-(α-D-ribofuranosyl)-2-bromopyridine, 5-(α-D-ribo-furanosyl)-2-bromopyridine, 6-(α-D-ribofuranosyl)-2-bromo-pyridine, 6-(α-D-ribofuranosyl)-3-bromopyridine, 4-(α-D-ribofuranosyl)-3-bromopyridine, 2-(α-D-ribofuranosyl)-3-bromo-pyridine, 2-(α-D-ribofuranosyl)-4-bromopyridine, 3-(α-D-ribofuranosyl)-4-bromopyridine, 4-(α-D-ribofuranosyl)-2-chloropyridine, 3-(α-D-ribo-furanosyl)-2-chloropyridine, 5-(α-D-ribo-furanosyl)-2-chloropyridine, 6-(α-D-ribo-furanosyl)-2-chloropyridine, 6-(α-D-ribofuranosyl)-3-chloropyridine, 5-(α-D-ribo-furanosyl)-3-chloropyridine, 4-(α-D-ribofuranosyl)-3-chloro-pyridine, 2-(α-D-ribo-furanosyl)-3-chloropyridine, 2-(α-D-ribofuranosyl)-4-chloropyridine, 3-(α-D-ribo-furanosyl)-4-chloropyridine, 4-(α-D-ribofuranosyl)-2-iodopyridine, 3-(α-D-ribo-furanosyl)-2-iodopyridine, 5-(α-D-ribofuranosyl)-2-iodopyridine, 6-(α-D-ribofuranosyl)-2-iodo-pyridine, 6-(α-D-ribofuranosyl)-3-iodopyridine, 5-(α-D-ribofuranosyl)-3-iodopyridine, 4-(α-D-ribofuranosyl)-3-iodopyridine, 2-(α-D-ribofuranosyl)-3-iodopyridine, 2-(α-D-ribo-furanosyl)-4-iodopyridine, 3-(α-D-ribofuranosyl)-4-iodopyridine, 4-(α-D-ribofuranosyl)-2-cyanopyridine, 3-(α-D-ribofuranosyl)-2-cyanopyridine, 5-(α-D-ribofuranosyl)-2 cyano-pyridine, 6-(α-D-ribofuranosyl)-2-cyanopyridine, 5-(α-D-ribofuranosyl)-3-cyanopyridine, 6-(α-D-ribofuranosyl)-3-cyanopyridine, 4-(α-D-ribofuranosyl)-3-cyanopyridine, 2-(α-D-ribofuranosyl)-3-cyanopyridine, 2-(α-D-ribofuranosyl)-4-cyanopyridine, 3-(α-D-ribo-furanosyl)-4-cyanopyridine, methyl6-(α-D-ribofuranosyl)nicotinate, methyl 4-(α-D-ribo-furanosyl)nicotinate, methyl 2-(α-D-ribofuranosyl)nicotinate, methyl 4-(α-D-ribo-furanosyl)picolinate, methyl 3-(α-D-ribofuranosyl)picolinate, methyl 5-(α-D-ribo-furanosyl)picolinate, methyl 6-(α-D-ribofuranosyl)picolinate, methyl 2-(α-D-ribo-furanosyl)isonicotinate, methyl 3-(α-D-ribo-furanosyl)isonicotinate, methyl 6-(α-D-ribo-furanosyl)picotinate, methyl 4-(α-D-ribofuranosyl)nicotinate, 2-(α-D-ribofuranosyl)-nicotinamide, 4-(α-D-ribofuranosyl )-picolinamide, 3-(α-D-ribofuranosyl)picolinamide, 5-(α-D-ribofuranosyl)picolinamide, 6-(α-D-ribofuranosyl)picolinamide, 2-(α-D-ribo-furanosyl)isonicotinamide, and 3-(α-D-ribo-furanosyl)isonicotinamide.

By following the same procedures but using the corresponding altro/allo mixtures of (2,4;3,5-di-O-benzylidene-L-hexityl)pyridines as the starting material, the L-nucleoside counterparts of the above C-nucleosides are also prepared.

### Example 6

### 5-(β-D-Ribofuranosyl)nicotinamide (II, R⁵═CONH₂, W₆═N, W₅═W₇═W₈═CH) and 5-(α-D-ribofuranosyl)nicotinamide (17, R⁵═CONH₂, W₆═N, W₅═W₇═W₈═CH)—an Alternative Synthesis

A solution of an altro/allo isomeric mixture of 5-(2,4;3,5-di-O-benzylidene-D-hexityl)-3-cyanopyridine (80 mg, 0.19 mmol) in a mixture of water and methanol (10 mL, 1:1) is refluxed for 1.5 hours with Amberlite 400 (OH⁻) (3 g). The resin is removed by filtration and the filtrate is concentrated to dryness in vacuo. The residue is purified by silica gel column chromatography using chloroform-methanol (95:5 v/v) as the eluent. 5-(2,4;3,5-di-O-benzylidene-D-hexityl)-nicotinamide is obtained as an altro/allo mixture, which is identical with an authentic sample prepared according to the procedure as described in Example 3.

By following the procedure of Example 6 but using the corresponding cyanopyrimidine derivatives, the following carboxamides are also synthesized:

4-(2,4;3,5-di-O-benzylidene-D-hexityl)picolinamide, 3-(2,4;3,5-di-O-benzylidene-D-hexityl)picolinamide, 5-(2,4;3,5-di-O-benzylidene-D-hexityl)picolinamide, 6-(2,4;3,5-di-O-benzylidene-D-hexityl)picolinamide, 6-(2,4;3,5-Di-O-benzylidene-D-hexityl)-nicotinamide, 4-(2,4;3,5-Di-O-benzylidene-D-hexityl)nicotinamide, 2-(2,4;3,5-Di-O-benzylidene-D-hexityl)nicotinamide, 2-(2,4;3,5-Di-O-benzylidene-D-hexityl)isonicotinamide, and 3-(2,4;3,5-Di-O-benzylidene-D-hexityl)isonicotinamide.

These carboxamides synthesized by the procedure described in Example 6 are identical with the corresponding samples prepared by following the procedure of Example 3.

### Example 7

### Separation of altro- and allo-isomers

A crude altro/allo isomeric mixture of 6-(2,4;3,5-di-O-benzylidene-D-hexityl)-2-bromopyridine prepared by condensation of 2,6-dibromopyridine (2.18 g, 9.20 mmol) and 2,4,3,5-di-O-benzylidene-D-aldehydo-ribose (1.0 g, 3.06 mmol) according to the procedure of Example 1 is placed on a silica gel column, which is washed with n-hexane-ethyl acetate (94:6). 6-(2,4,3,5-Di-O-benzylidene-D-altrityl)-2-bromopyridine (342 mg, 23%) is eluted first. The column is then washed with n-hexane-ethyl acetate (92:8) solvent system, which elutes 6-(2,4,3,5-di-O-benzylidene-D-allityl)-2-bromopyridine (282 mg, 19%). Both isomers are obtained as foams.

¹H NMR for the altrityl isomer (CDCl₃): δ 7.55-7.05 (13H, m, aromatic H), 5.77, 5.49 (2H, 2s, benzylidene-CH<), 5.14 (1H, q, collapsed to d on addition of D₂O, H-1′, J_{1′,2′}=3.02 Hz), 4.40 (2H, m, H-2′,3′), 3.92 (3H, m, H-4′,5′,5″).

¹H NMR for the allityl isomer (CDCl₃): δ 7.49-7.24 (13H, m, aromatic H), 5.66, (2H, 2s, benzylidene-CH<), 5.0 (1H, broad s, H-1′), 4.32 (2H, m, H-2′,3′), 3.97 (3H, m, H-4′,5′,5″).

By following the same procedure but using the corresponding (2,4;3,5-di-O-benzylidene-D-hexityl)pyridines, the following derivatives are obtained:

4-(2,4;3,5-Di-O-benzylidene-D-altrityl)-2-bromopyridine,

4-(2,4;3,5-Di-O-benzylidene-D-allityl)-2-bromopyridine,

3-(2,4;3,5-Di-O-benzylidene-D-altrityl)-2-bromopyridine,

3-(2,4;3,5-Di-O-benzylidene-D-allityl)-2-bromopyridine,

3-(2,4;3,5-Di-O-benzylidene-D-altrityl)-2-bromopyridine,

3-(2,4;3,5-Di-O-benzylidene-D-allityl)-2-bromopyridine,

6-(2,4;3,5-Di-O-benzylidene-D-altrityl)-3-bromopyridine,

6-(2,4;3,5-Di-O-benzylidene-D-allityl)-3-bromopyridine,

5-(2,4;3,5-Di-O-benzylidene-D-altrityl)-3-bromopyridine,

5-(2,4;3,5-Di-O-benzylidene-D-allityl)-3-bromopyridine,

4-(2,4;3,5-Di-O-benzylidene-D-altrityl)-3-bromopyridine,

4-(2,4;3,5-Di-O-benzylidene-D-allityl)-3-bromopyridine,

2-(2,4;3,5-Di-O-benzylidene-D-altrityl)-3-bromopyridine,

2-(2,4;3,5-Di-O-benzylidene-D-allityl)-3-bromopyridine,

2-(2,4;3,5-Di-O-benzylidene-D-altrityl)-4-bromopyridine,

2-(2,4;3,5-Di-O-benzylidene-D-allityl)-4-bromopyridine,

3-(2,4;3,5-Di-O-benzylidene-D-altrityl)-4-bromopyridine,

3-(2,4;3,5-Di-O-benzylidene-D-allityl)-4-bromopyridine,

4-(2,4;3,5-Di-O-benzylidene-D-altrityl)-2-chloropyridine,

4-(2,4;3,5-Di-O-benzylidene-D-allityl)-2-chloropyridine,

3-(2,4;3,5-Di-O-benzylidene-D-altrityl)-2-chloropyridine,

3-(2,4;3,5-Di-O-benzylidene-D-allityl)-2-chloropyridine,

5-(2,4;3,5-Di-O-benzylidene-D-altrityl)-2-chloropyridine,

5-(2,4;3,5-Di-O-benzylidene-D-allityl)-2-chloropyridine,

6-(2,4;3,5-Di-O-benzylidene-D-altrityl)-2-chloropyridine,

6-(2,4;3,5-Di-O-benzylidene-D-allityl)-2-chloropyridine,

6-(2,4;3,5-Di-O-benzylidene-D-altrityl)-3-chloropyridine,

6-(2,4;3,5-Di-O-benzylidene-D-allityl)-3-chloropyridine,

5-(2,4;3,5-Di-O-benzylidene-D-altrityl)-3-chloropyridine,

5-(2,4;3,5-Di-O-benzylidene-D-allityl)-3-chloropyridine,

4-(2,4;3,5-Di-O-benzylidene-D-altrityl)-3-chloropyridine,

4-(2,4;3,5-Di-O-benzylidene-D-allityl)-3-chloropyridine,

2-(2,4;3,5-Di-O-benzylidene-D-altrityl)-3-chloropyridine,

2-(2,4;3,5-Di-O-benzylidene-D-allityl)-3-chloropyridine,

2-(2,4;3,5-Di-O-benzylidene-D-altrityl)-4-chloropyridine,

2-(2,4;3,5-Di-O-benzylidene-D-allityl)-4-chloropyridine,

3-(2,4;3,5-Di-O-benzylidene-D-altrityl)-4-chloropyridine,

3-(2,4;3,5-Di-O-benzylidene-D-allityl)-4-chloropyridine,

4-(2,4;3,5-Di-O-benzylidene-D-altrityl)-2-iodopyridine,

4-(2,4;3,5-Di-O-benzylidene-D-allityl)-2-iodopyridine,

3-(2,4;3,5-Di-O-benzylidene-D-altrityl)-2-iodopyridine,

3-(2,4;3,5-Di-O-benzylidene-D-allityl)-2-iodopyridine,

5-(2,4;3,5-Di-O-benzylidene-D-altrityl)-2-iodopyridine,

5-(2,4;3,5-Di-O-benzylidene-D-allityl)-2-iodopyridine,

6-(2,4;3,5-Di-O-benzylidene-D-altrityl)-2-iodopyridine,

6-(2,4;3,5-Di-O-benzylidene-D-allityl)-2-iodopyridine,

6-(2,4;3,5-Di-O-benzylidene-D-altrityl)-3-iodopyridine,

6-(2,4;3,5-Di-O-benzylidene-D-allityl)-3-iodopyridine,

5-(2,4;3,5-Di-O-benzylidene-D-altrityl)-3-iodopyridine,

5-(2,4;3,5-Di-O-benzylidene-D-allityl)-3-iodopyridine,

4-(2,4;3,5-Di-O-benzylidene-D-altrityl)-3-iodopyridine,

4-(2,4;3,5-Di-O-benzylidene-D-allityl)-3-iodopyridine,

2-(2,4;3,5-Di-O-benzylidene-D-altrityl)-3-iodopyridine,

2-(2,4;3,5-Di-O-benzylidene-D-allityl)-3-iodopyridine,

2-(2,4;3,5-Di-O-benzylidene-D-altrityl)-4-iodopyridine,

2-(2,4;3,5-Di-O-benzylidene-D-allityl)-4-iodopyridine,

3-(2,4;3,5-Di-O-benzylidene-D-altrityl)-4-iodopyridine,

3-(2,4;3,5-Di-O-benzylidene-D-allityl)-4-iodopyridine,

4-(2,4;3,5-Di-O-benzylidene-D-altrityl)-2-cyanopyridine,

4-(2,4;3,5-Di-O-benzylidene-D-allityl)-2-cyanopyridine,

3-(2,4;3,5-Di-O-benzylidene-D-altrityl)-2-cyanopyridine,

3-(2,4;3,5-Di-O-benzylidene-D-allityl)-2-cyanopyridine,

5-(2,4;3,5-Di-O-benzylidene-D-altrityl)-2-cyanopyridine,

5-(2,4;3,5-Di-O-benzylidene-D-allityl)-2-cyanopyridine,

6-(2,4;3,5-Di-O-benzylidene-D-altrityl)-2-cyanopyridine,

6-(2,4;3,5-Di-O-benzylidene-D-allityl)-2-cyanopyridine,

5-(2,4;3,5-Di-O-benzylidene-D-altrityl)-3-cyanopyridine,

5-(2,4;3,5-Di-O-benzylidene-D-allityl)-3-cyanopyridine,

6-(2,4;3,5-Di-O-benzylidene-D-altrityl)-3-cyanopyridine,

6-(2,4;3,5-Di-O-benzylidene-D-allityl)-3-cyanopyridine,

4-(2,4;3,5-Di-O-benzylidene-D-altrityl)-3-cyanopyridine,

4-(2,4;3,5-Di-O-benzylidene-D-allityl)-3-cyanopyridine,

2-(2,4;3,5-Di-O-benzylidene-D-altrityl)-3-cyanopyridine,

2-(2,4;3,5-Di-O-benzylidene-D-allityl)-3-cyanopyridine,

3-(2,4;3,5-Di-O-benzylidene-D-altrityl)-4-cyanopyridine,

3-(2,4;3,5-Di-O-benzylidene-D-allityl)-4-cyanopyridine,

3-(2,4;3,5-Di-O-benzylidene-D-altrityl)-4-cyanopyridine, and

3-(2,4;3,5-Di-O-benzylidene-D-allityl)-4-cyanopyridine.

### Example 8

### Methyl 6-(2,4;3,5-di-O-benzylidene-D-altrityl)picolinate (13, R═H, R⁵═CO₂CH₃, W₆═N, W₅═W₇═W₈═CH)

A solution of 6-(2,4;3,5-di-O-benzylidene-D-altrityl)-2-bromopyridine (200 mg, 0.43 mmol) in hexamethylphosphoric triamide (0.25 mL) and ethyl ether (5 mL) is treated at −78° C. with butyllithium (2 mL of 2.5 M in n-hexane). After stirring for 15 minutes at −78° C., excess solic carbon dioxide is added and the mixture is allowed to warm to room temperature while stirring. The mixture is neutralized with 1N hydrochloric acid, and then the product is extracted with ethyl ether (15 mL×2). The extracts are combined, washed with brine (20 mL×3), dried over sodium sulfate, filtered, and the filtrate treated with ethereal solution of diazomethane. Excess diazomethane is decomposed by addition of acetic acid, and then the mixture is concentrated in vacuo. The residue is chromatographed on a silica gel column using n-hexane-ethyl acetate (75:25). Methyl 6-(2,4;3,5-di-O-benzylidene-D-altrityl)picolinate (122 mg, 64%) is obtained as colorless crystals, mp 176-179° C. (after recrystallization from ethyl ether). ¹H NMR (CDCl₃): δ 8.07-7.31 (13H, m, aromatic), 5.64, 5.67 (2H, 2s, benzylidene-CH<), 5.25 (1H, broad s, H-1′), 4.43-3.95 (5H, H-2′,3′,4′,5′,5″), 3.95 (3H, s, CH₃ ester).

By following the same procedure but using the corresponding (2,4;3,5-di-O-benzylidene-D-altrityl)bromopyrimidines as the starting materials, the following methyl esters are prepared:

Methyl 6-(2,4;3,5-di-O-benzylidene-D-altrityl)nicotinate, Methyl 5-(2,4;3,5-di-O-benzylidene-D-altrityl)nicotinate, Methyl 4-(2,4;3,5-di-O-benzylidene-D-altrityl)nicotinate, Methyl 2-(2,4;3,5-di-O-benzylidene-D-altrityl)nicotinate, Methyl 4-(2,4;3,5-di-O-benzylidene-D-altrityl)-picolinate, Methyl 3-(2,4;3,5-di-O-benzylidene-D-altrityl)picolinate, Methyl 5-(2,4;3,5-di-O-benzylidene-D-altrityl)picolinate, Methyl 2-(2,4;3,5-di-O-benzylidene-D-altrityl)isonicotinate and Methyl 3-(2,4;3,5-di-O-benzylidene-D-altrityl)isonicotinate.

By following the same procedure but using the corresponding (2,4;3,5-di-O-benzylidene-D-allityl)bromopyrimidines as the starting materials, the following methyl esters are prepared:

Methyl 6-(2,4;3,5-di-O-benzylidene-D-allityl)nicotinate, Methyl 5-(2,4;3,5-di-O-benzylidene-D-allityl)nicotinate, Methyl 4-(2,4;3,5-di-O-benzylidene-D-allityl)nicotinate, Methyl 2-(2,4;3,5-di-O-benzylidene-D-allityl)nicotinate, Methyl 4-(2,4;3,5-di-O-benzylidene-D-allityl)picolinate, Methyl 3-(2,4;3,5-di-O-benzylidene-D-allityl)picolinate, Methyl 5-(2,4;3,5-di-O-benzylidene-D-allityl)picolinate, Methyl 6-(2,4;3,5-di-O-benzylidene-D-allityl)picolinate, Methyl 2-(2,4;3,5-di-O-benzylidene-D-allityl)isonicotinate and Methyl 3-(2,4;3,5-di-O-benzylidene-D-allityl)-iso-nicotinate.

### Example 9

### 6-(2,4;3,5-Di-O-benzylidene-D-allityl)picolinamide (14, R═H, R⁵═CONH₂, W₆═N, W₅═W₇═W₈═CH)

Methyl 6-(2,4;3,5-di-O-benzylidene-D-allityl)picolinate (75 mg, 0.16 mmol) is dissolved in a 4:1 mixture of methanol and ammonia containing a catalytic amount of sodium hydride (ca. 2 mg). The solution is stirred at room temperature for 20 hours, and then is concentrated to dryness in vacuo. The residue is purified by chromatography on a silica gel column. 6-(2,4;3,5-Di-O-benzylidene-D-allityl)picolinamide is obtained as colorless crystals, 65 mg (89%), mp 201-203° C. (recrystallization from n-hexane-methanol).

¹H NMR (CDCl₃): δ 8.07-7.11 (13H, m, aromatic), 5.78, 5.51 (2H, 2s, benzylidene-CH-Ph), 5.19 (1H, q, H-1′, collapsed to a doublet upon addition of D₂O, J_{1′,2′}=3.29 Hz), 4.40 (2H, m, H-2′,3′), 3.91 (3H, m, H-4′,5′,5″).

By following the same procedure but using the corresponding (2,4;3,5-di-O-benzilidene-L-altrityl)bromopyridines as the starting materials, the L-counterparts of the above compounds are prepared.

By following the same procedure but using the corresponding methyl esters, the following carboxamides are prepared:

6-(2,4;3,5-Di-O-benzylidene-D-allityl)nicotinamide, 5-(2,4;3,5-Di-O-benzylidene-D-allityl)-nicotinamide, 4-(2,4;3,5-Di-O-benzylidene-D-allityl)nicotinamide, 2-(2,4;3,5-Di-0-benzylidene-D-allityl)nicotinamide, 4-(2,4;3,5-Di-O-benzylidene-D-allityl)picolinamide, 3-(2,4;3,5-Di-O-benzylidene-D-allityl)picolinamide, 5-(2,4;3,5-Di-O-benzylidene-D-allityl)-picolinamide, 6-(2,4;3,5-Di-O-benzylidene-D-allityl)picolinamide, 2-(2,4;3,5-Di-O-benzylidene-D-allityl)-iso-nicotinamide, 3-(2,4;3,5-Di-O-benzylidene-D-allityl)-iso-nicotinamide, 6-(2,4;3,5-Di-O-benzylidene-D-altrityl)nicotinamide, 5-(2,4;3,5-Di-O-benzylidene-D-altrityl)nicotinamide, 4-(2,4;3,5-Di-O-benzylidene-D-altrityl)nicotinamide, 2-(2,4;3,5-Di-O-benzylidene-D-altrityl)nicotinamide, 4-(2,4;3,5-Di-O-benzylidene-D-altrityl)picolinamide, 3-(2,4;3,5-Di-O-benzylidene-D-altrityl)-picolinamide, 5-(2,4;3,5-di-O-benzylidene-D-altrityl)picolinamide, 6-(2,4;3,5-di-O-benzylidene-D-altrityl)picolinamide, 2-(2,4;3,5-di-O-benzylidene-D-altrityl)isonicotinamide and 3-(2,4;3,5-di-O-benzylidene-D-altrityl)isonicotinamide.

By following the same procedure but using the corresponding (2,4;3,5-di-O-benzylidene-L-allityl)bromopyridines as the starting materials, the L-counterparts of the above compounds are prepared.

### Example 10

### 6-(2,4;3,5-Di-O-benzylidene-1-O-mesyl-D-altrityl)picolinamide (15, R=Ms, R⁵═CONH₂, W₆═N, W₅═W₇═W₈═CH)

A solution of 6-(2,4;3,5-di-O-benzylidene-D-altrityl)picolinamide (50 mg, 0.11 mmol), triethylamine (150 μL, 1.08 mmol) and 4-dimethylaminopyridine (ca. 3 mg) in methylene chloride (4 mL) is treated at room temperature with mesyl chloride (40 μL, 0.52 mmol). The mixture is stirred for 30 minutes at room temperature, and then concentrated in vacuo to dryness. The residue is chromatographed on a silica gel column using methylene chloride-methanol (98:2) as the eluent. 6-(2,4;3,5-Di-O-benzylidene-1-O-mesyl-D-altrityl)picolinamide (49 mg, 83%) is obtained after recrystallization from n-hexane-ethyl ether, mp 109-110° C. ¹H NMR (CDCl₃): δ 8.20-7.34 (13H, m, aromatic), 6.03 (1H, d, H-1′, J_{1′,2′}=2.47 Hz), 5.68, 5.61 (2H, 2s, benzylidene-CH<), 4.48-3.94 (5H, H-2′,3′,4′,5′,5″), 3.02 (3H, s, mesyl CH₃).

By following the same procedure but using the corresponding (2,4;3,5-di-O-benzylidene-D-altrityl)pyridines, the following mesyl derivatives are prepared:

4-(2,4;3,5-Di-O-benzylidene-1-O-mesyl-D-altrityl)-2-bromopyridine,

3-(2,4;3,5-Di-O-benzylidene-1-O-mesyl-D-altrityl)-2-bromopyridine,

5-(2,4;3,5-Di-O-benzylidene-1-O-mesyl-D-altrityl)-2-bromopyridine,

6-(2,4;3,5-Di-O-benzylidene-1-O-mesyl-D-altrityl)-2-bromopyridine,

6-(2,4;3,5-Di-O-benzylidene-1-O-mesyl-D-altrityl)-3-bromopyridine,

4-(2,4;3,5-Di-O-benzylidene-1-O-mesyl-D-altrityl)-3-bromopyridine,

2-(2,4;3,5-Di-O-benzylidene-1-O-mesyl-D-altrityl)-3-bromopyridine,

2-(2,4;3,5-Di-O-benzylidene-1-O-mesyl-D-altrityl)-4-bromopyridine,

3-(2,4;3,5-Di-O-benzylidene-1-O-mesyl-D-altrityl)-4-bromopyridine,

4-(2,4;3,5-Di-O-benzylidene-1-O-mesyl-D-altrityl)-2-chloropyridine,

3-(2,4;3,5-Di-O-benzylidene-1-O-mesyl-D-altrityl)-2-chloropyridine,

5-(2,4;3,5-Di-O-benzylidene-1-O-mesyl-D-altrityl)-2-chloropyridine,

6-(2,4;3,5-Di-O-benzylidene-1-O-mesyl-D-altrityl)-2-chloropyridine,

6-(2,4;3,5-Di-O-benzylidene-1-O-mesyl-D-altrityl)-3-chloropyridine,

5-(2,4;3,5-Di-O-benzylidene-1-O-mesyl-D-altrityl)-3-chloropyridine,

4-(2,4;3,5-Di-O-benzylidene-1-O-mesyl-D-altrityl)-3-chloropyridine,

2-(2,4;3,5-Di-O-benzylidene-1-O-mesyl-D-altrityl)-3-chloropyridine,

2-(2,4;3,5-Di-O-benzylidene-1-O-mesyl-D-altrityl)-4-chloropyridine,

3-(2,4;3,5-Di-O-benzylidene-1-O-mesyl-D-altrityl)-4-chloropyridine,

4-(2,4;3,5-Di-O-benzylidene-1-O-mesyl-D-altrityl)-2-iodopyridine,

3-(2,4;3,5-Di-O-benzylidene-1-O-mesyl-D-altrityl)-2-iodopyridine,

5-(2,4;3,5-Di-O-benzylidene-1-O-mesyl-D-altrityl)-2-iodopyridine,

6-(2,4;3,5-Di-O-benzylidene-1-O-mesyl-D-altrityl)-2-iodopyridine,

6-(2,4;3,5-Di-O-benzylidene-1-O-mesyl-D-altrityl)-3-iodopyridine,

5-(2,4;3,5-Di-O-benzylidene-1-O-mesyl-D-altrityl)-3-iodopyridine,

4-(2,4;3,5-Di-O-benzylidene-1-O-mesyl-D-altrityl)-3-iodopyridine,

2-(2,4;3,5-Di-O-benzylidene-1-O-mesyl-D-altrityl)-3-iodopyridine,

2-(2,4;3,5-Di-O-benzylidene-1-O-mesyl-D-altrityl)-4-iodopyridine,

3-(2,4;3,5-Di-O-benzylidene-1-O-mesyl-D-altrityl)-4-iodopyridine,

4-(2,4;3,5-Di-O-benzylidene-1-O-mesyl-D-altrityl)-2-cyanopyridine,

3-(2,4;3,5-Di-O-benzylidene-1-O-mesyl-D-altrityl)-2-cyanopyridine,

5-(2,4;3,5-Di-O-benzylidene-1-O-mesyl-D-altrityl)-2-cyanopyridine,

6-(2,4;3,5-Di-O-benzylidene-1-O-mesyl-D-altrityl)-2-cyanopyridine,

5-(2,4;3,5-Di-O-benzylidene-1-O-mesyl-D-altrityl)-3-cyanopyridine,

6-(2,4;3,5-Di-O-benzylidene-1-O-mesyl-D-altrityl)-3-cyanopyridine,

4-(2,4;3,5-Di-O-benzylidene-1-O-mesyl-D-altrityl)-3-cyanopyridine,

2-(2,4;3,5-Di-O-benzylidene-1-O-mesyl-D-altrityl)-3-cyanopyridine,

2-(2,4;3,5-Di-O-benzylidene-1-O-mesyl-D-altrityl)-4-cyanopyridine,

3-(2,4;3,5-Di-O-benzylidene-1-O-mesyl-D-altrityl)-4-cyanopyridine,

Methyl 6-(2,4;3,5-di-O-benzylidene-1-O-mesyl-D-altrityl)nicotinate,

Methyl 4-(2,4;3,5-di-O-benzylidene-1-O-mesyl-D-altrityl)nicotinate,

Methyl 2-(2,4;3,5-di-O-benzylidene-1-O-mesyl-D-altrityl)nicotinate,

Methyl 4-(2,4;3,5-di-O-benzylidene-1-O-mesyl-D-altrityl)picolinate,

Methyl 3-(2,4;3,5-di-O-benzylidene-1-O-mesyl-D-altrityl)picolinate,

Methyl 5-(2,4;3,5-di-O-benzylidene-1-O-mesyl-D-altrityl)picolinate,

Methyl 6-(2,4;3,5-di-O-benzylidene-1-O-mesyl-D-altrityl)picolinate,

Methyl 2-(2,4;3,5-di-O-benzylidene-1-O-mesyl-D-altrityl)isonicotinate,

Methyl 3-(2,4;3,5-di-O-benzylidene-1-O-mesyl-D-altrityl)isonicotinate,

2-(2,4;3,5-Di-O-benzylidene-1-O-mesyl-D-altrityl)nicotinamide,

4-(2,4;3,5-Di-O-benzylidene-1-O-mesyl-D-altrityl)picolinamide,

3-(2,4;3,5-Di-O-benzylidene-1-O-mesyl-D-altrityl)picolinamide,

5-(2,4;3,5-Di-O-benzylidene-1-O-mesyl-D-altrityl)picolinamide,

6-(2,4;3,5-Di-O-benzylidene-1-O-mesyl-D-altrityl)picolinamide,

2-(2,4;3,5-Di-O-benzylidene-1-O-mesyl-D-altrityl)isonicotinamide, and

3-(2,4;3,5-Di-O-benzylidene-1-O-mesyl-D-altrityl)isonicotinamide.

By following the same procedure but using the corresponding (2,4;3,5-di-O-benzylidene-L-allityl)bromopyridines as the starting materials, the L-counterparts of the above compounds are prepared.

By following the same procedure but using the corresponding (2,4;3,5-di-O-benzylidene-D-allityl)pyridines, the following compounds are synthesized:

4-(2,4;3,5-Di-O-benzylidene-1-O-mesyl-D-allityl)-2-bromopyridine,

3-(2,4;3,5-Di-O-benzylidene-1-O-mesyl-D-allityl)-2-bromopyridine,

5-(2,4;3,5-Di-O-benzylidene-1-O-mesyl-D-allityl)-2-bromopyridine,

6-(2,4;3,5-Di-O-benzylidene-1-O-mesyl-D-allityl)-2-bromopyridine,

6-(2,4;3,5-Di-O-benzylidene-1-O-mesyl-D-allityl)-3-bromopyridine,

4-(2,4;3,5-Di-O-benzylidene-1-O-mesyl-D-allityl)-3-bromopyridine,

2-(2,4;3,5-Di-O-benzylidene-1-O-mesyl-D-allityl)-3-bromopyridine,

2-(2,4;3,5-Di-O-benzylidene-1-O-mesyl-D-allityl)-4-bromopyridine,

3-(2,4;3,5-Di-O-benzylidene-1-O-mesyl-D-allityl)-4-bromopyridine,

4-(2,4;3,5-Di-O-benzylidene-1-O-mesyl-D-allityl)-2-chloropyridine,

3-(2,4;3,5-Di-O-benzylidene-1-O-mesyl-D-allityl)-2-chloropyridine,

5-(2,4;3,5-Di-O-benzylidene-1-O-mesyl-D-allityl)-2-chloropyridine,

6-(2,4;3,5-Di-O-benzylidene-1-O-mesyl-D-allityl)-2-chloropyridine,

6-(2,4;3,5-Di-O-benzylidene-1-O-mesyl-D-allityl)-3-chloropyridine,

5-(2,4;3,5-Di-O-benzylidene-1-O-mesyl-D-allityl)-3-chloropyridine,

4-(2,4;3,5-Di-O-benzylidene-1-O-mesyl-D-allityl)-3-chloropyridine,

2-(2,4;3,5-Di-O-benzylidene-1-O-mesyl-D-allityl)-3-chloropyridine,

2-(2,4;3,5-Di-O-benzylidene-1-O-mesyl-D-allityl)-4-chloropyridine,

3-(2,4;3,5-Di-O-benzylidene-1-O-mesyl-D-allityl)-4-chloropyridine,

4-(2,4;3,5-Di-O-benzylidene-1-O-mesyl-D-allityl)-2-iodopyridine,

3-(2,4;3,5-Di-O-benzylidene-1-O-mesyl-D-allityl)-2-iodopyridine,

5-(2,4;3,5-Di-O-benzylidene-1-O-mesyl-D-allityl)-2-iodopyridine,

6-(2,4;3,5-Di-O-benzylidene-1-O-mesyl-D-allityl)-2-iodopyridine,

6-(2,4;3,5-Di-O-benzylidene-1-O-mesyl-D-allityl)-3-iodopyridine,

5-(2,4;3,5-Di-O-benzylidene-1-O-mesyl-D-allityl)-3-iodopyridine,

4-(2,4;3,5-Di-O-benzylidene-1-O-mesyl-D-allityl)-3-iodopyridine,

2-(2,4;3,5-Di-O-benzylidene-1-O-mesyl-D-allityl)-3-iodopyridine,

2-(2,4;3,5-Di-O-benzylidene-1-O-mesyl-D-allityl)-4-iodopyridine,

3-(2,4;3,5-Di-O-benzylidene-1-O-mesyl-D-allityl)-4-iodopyridine,

4-(2,4;3,5-Di-O-benzylidene-1-O-mesyl-D-allityl)-2-cyanopyridine,

3-(2,4;3,5-Di-O-benzylidene-1-O-mesyl-D-allityl)-2-cyanopyridine,

5-(2,4;3,5-Di-O-benzylidene-1-O-mesyl-D-allityl)-2-cyanopyridine,

6-(2,4;3,5-Di-O-benzylidene-1-O-mesyl-D-allityl)-2-cyanopyridine,

5-(2,4;3,5-Di-O-benzylidene-1-O-mesyl-D-allityl)-3-cyanopyridine,

6-(2,4;3,5-Di-O-benzylidene-1-O-mesyl-D-allityl)-3-cyanopyridine,

4-(2,4;3,5-Di-O-benzylidene-1-O-mesyl-D-allityl)-3-cyanopyridine,

2-(2,4;3,5-Di-O-benzylidene-1-O-mesyl-D-allityl)-3-cyanopyridine,

2-(2,4;3,5-Di-O-benzylidene-1-O-mesyl-D-allityl)-4-cyanopyridine,

3-(2,4;3,5-Di-O-benzylidene-1-O-mesyl-D-allityl)-4-cyanopyridine,

Methyl 6-(2,4;3,5-di-O-benzylidene-1-O-mesyl-D-allityl)nicotinate,

Methyl 4-(2,4;3,5-di-O-benzylidene-1-O-mesyl-D-allityl)nicotinate,

Methyl 2-(2,4;3,5-di-O-benzylidene-1-O-mesyl-D-allityl)nicotinate,

Methyl 4-(2,4;3,5-di-O-benzylidene-1-O-mesyl-D-allityl)picolinate,

Methyl 3-(2,4;3,5-di-O-benzylidene-1-O-mesyl-D-allityl)picolinate,

Methyl 5-(2,4;3,5-di-O-benzylidene-1-O-mesyl-D-allityl)picolinate,

Methyl 6-(2,4;3,5-di-O-benzylidene-1-O-mesyl-D-allityl)picolinate,

Methyl 2-(2,4;3,5-di-O-benzylidene-1-O-mesyl-D-allityl)isonicotinate,

Methyl 3-(2,4;3,5-di-O-benzylidene-1-O-mesyl-D-allityl)isonicotinate,

2-(2,4;3,5-Di-O-benzylidene-1-O-mesyl-D-allityl)nicotinamide,

4-(2,4;3,5-Di-O-benzylidene-1-O-mesyl-D-allityl)picolinamide,

3-(2,4;3,5-Di-O-benzylidene-1-O-mesyl-D-allityl)picolinamide,

5-(2,4;3,5-Di-O-benzylidene-1-O-mesyl-D-allityl)picolinamide,

6-(2,4;3,5-Di-O-benzylidene-1-O-mesyl-D-allityl)picolinamide,

2-(2,4;3,5-Di-O-benzylidene-1-O-mesyl-D-allityl)isonicotinamide, and

3-(2,4;3,5-Di-O-benzylidene-1-O-mesyl-D-allityl)isonicotinamide.

By following the same procedure but using the corresponding (2,4;3,5-di-O-benzylidene-L-allityl)pyridines as the starting material, the L-counterparts of the above compounds are prepared.

### Example 11

### 5-(β-D-Ribofuranosyl)-3-cyanopyridine ([II], R⁵═CN W₆═N, W₅═W₇═W₈═CH)

5-(2,4;3,5-Di-O-benzylidene-1-O-mesyl-D-altrityl)-3-cyanopyridine (28 mg, 0.06 mmol) is dissolved in a 4:1 mixture of trifluoroacetic acid and chloroform (4 mL). After stirring for 15 minutes at room temperature, the reaction is quenched by addition of water (10 mL). The aqueous layer is separated, extracted five times with ethyl ether (10 mL each), and then concentrated in vacuo. The residue is chromatographed on a silica gel column using methylene chloride-methanol as the eluent. 5-(β-D-Ribofuranosyl)-3-cyanopyridine (8 mg, 57%) is obtained as colorless crystals, mp 169-171° C. (after recrystallization from ethanol). ¹H NMR (Me₂SO-d₆): δ 8.93 (1H, d, H-2, spacing 1.93 Hz), 8.86 (1H, d, H-6, spacing 1.93 Hz), 8.31 (1H, m, H-4), 4.93 (1H, d, H-1′, J_{1′,2′}=7.41 Hz), 3.99-3.50 (5H, m, H-2′,3′,4′,5′,5″).

By following the same procedure but using the corresponding (2,4;3,5-di-O-benzylidene-1-O-mesyl-D-altrityl)pyridines, all the (β-D-ribofuranosyl)pyrimidine C-nucleosides synthesized according to the procedure of Example 5 are prepared, and their spectral properties are identical to the corresponding β-C-nucleosides.

By following the same procedure but using the corresponding (2,4;3,5-di-O-benzylidene-1-O-mesyl-D-allityl)pyridines, all the (α-D-ribofuranosyl)pyrimidine C-nucleosides synthesized according to the procedure of Example 5 are obtained, and their spectral properties are identical to the corresponding (α-C-nucleosides.

By following the same procedure but using the corresponding (2,4;3,5-di-O-benzylidene-1-O-mesyl-y-altrityl)pyridines, all the (β-1-ribofuranosyl)pyrimidine C-nucleosides synthesized according to the procedure of Example 5 are prepared, and their spectral properties are identical to the corresponding β-C-nucleosides.

By following the same procedure but using the corresponding (2,4;3,5-di-O-benzylidene-1-O-mesyl-y-allityl)pyridines, all the (α-b-ribofuranosyl)pyrimidine C-nucleosides synthesized according to the procedure of Example 5 are obtained, and their spectral properties are identical to the corresponding α-C-nucleosides.

### Example 12

### P¹-[7-Hydroxy-5-methoxy-4-methylphthalan-1-on-6-(ethyl-2-yl)]-P²-(2′,3′-O-iso-propylideneadenosine-5′-yl)methylene-bis(phosphonate) ([I], R₁,R₂═O—C(CH₃)₂—O, R₃═R₄═H, Y═NH₂, Z=H, X═CH₂, R═[V])

To a solution of 2′,3′-O-isopropylideneadenosine-5′-methylenebis(phosphonate) ([I], R₁═R₂═OH, R₃═R₄═H, Y═NH₂, Z=H, X═CH₂,R═H) (700 mg, 1 mmol) in pyridine (4.0 mL) is added diisopropylcarbodiimide (7.8 mL, 5 mmol), and the mixture is shaken at room temperature. When three characteristic multi-signal resonances appear at −0.5-2.0 ppm, at 6.0-8.0 ppm and at 12.8-17.6 ppm, 6-(2-hydroxyethyl)-7-hydroxy-5-methoxy-4-methylphthalan-1-one (262 mg, 1.1 mmol) is added and the reaction is heated at 55-60° C. for 24 hours. The reaction is quenched by adding water (1 mL) and triethylamine (0.5 mL) when the ³¹P NMR spectrum of the reaction mixture exhibits two broad signals at 8 ppm and 25 ppm. The mixture is kept at 80-85° C. for 30 hours. HPLC purification on a Dynamax-300A C18-82-243-C column with a flow rate of 20 mL/min of 0.05 M triethylammonium bicarbonate (TEAB) followed by a linear gradient of 0.05 M TEAB-aq. Acetonitrile (70%) affords P¹-[7-hydroxy-5-methoxy-4-methylphthalan-1-on-6-(ethyl-2-yl)]-P²-(2′,3′-O-isopropylideneadenosine-5′-yl)methylene-bis-(phosphonate) ([I], R₁,R₂═O—C(CH₃)₂—O, R₃═R₄═H, Y═NH₂, Z=H, X═CH₂, R═[V]), 60 mg (32%) as the triethylammonium salt. ¹H NMR (D²O): δ 1.24 (18H, t, (CH₃CH₂)₃N), - - -

By following the same procedure but using the corresponding 2′,3′-O-protected nucleosides, the following compounds are synthesized:

P¹-[7-hydroxy-5-methoxy-4-methylphthalan-1-on-6-(ethyl-2-yl)]-P²-(2′,3′-O-isopropylideneinosine-5-yl)methylenebis(phosphonate),

P¹-[7-hydroxy-5-methoxy-4-methylphthalan-1-on-6-(ethyl-2-yl)]-P²-(2′,3′-O-isopropylideneguanosine-5-yl)methylenebis(phosphonate),

P¹-[7-hydroxy-5-methoxy-4-methylphthalan-1-on-6-(ethyl-2-yl)]-P²-(2′,3′-O-isopropylidenenebularine-5-yl)methylenebis(phosphonate),

P¹-[7-hydroxy-5-methoxy-4-methylphthalan-1-on-6-(ethyl-2-yl)]-P²-(2′,3′-O-isopropylidenexanthosine-5-yl)methylenebis(phosphonate),

P¹-[7-hydroxy-5-methoxy-4-methylphthalan-1-on-6-(ethyl-2-yl)]-P²-(2′,3′-O-isopropylidene-6-thioinosine-5-yl)methylenebis(phosphonate),

P¹-[7-hydroxy-5-methoxy-4-methylphthalan-1-on-6-(ethyl-2-yl)]-P²-(2′,3′-O-isopropylidene-6-thioguanosine-5-yl)methylenebis(phosphonate),

P¹-[7-hydroxy-5-methoxy-4-methylphthalan-1-on-6-(ethyl-2-yl)]-P²-(2′,3′-O-isopropylidene-O⁶-methylinosine-5-yl)methylenebis(phosphonate),

P¹-[7-hydroxy-5-methoxy-4-methylphthalan-1-on-6-(ethyl-2-yl)]-P²-(2′,3′-O-isopropylidene-O⁶-methylguanosine-5-yl)methylenebis(phosphonate),

P¹-[7-hydroxy-5-methoxy-4-methylphthalan-1-on-6-(ethyl-2-yl)]-P²-(2′,3′-O-isopropylidene-2-aminoadenosine-5-yl)methylenebis(phosphonate),

P¹-[7-hydroxy-5-methoxy-4-methylphthalan-1-on-6-(ethyl-2-yl)]-P²-(2′,3′-O-isopropylidene-2-fluoroadenosine-5-yl)methylenebis(phosphonate),

P¹-[7-hydroxy-5-methoxy-4-methylphthalan-1-on-6-(ethyl-2-yl)]-P²-(2′,3′-O-isopropylidene-2-chloroadenosine-5-yl)methylenebis(phosphonate),

P¹-[7-hydroxy-5-methoxy-4-methylphthalan-1-on-6-(ethyl-2-yl)]-P²-(2′,3′-O-isopropylidene-2-bromoadenosine-5-yl)methylenebis(phosphonate),

P¹-[7-hydroxy-5-methoxy-4-methylphthalan-1-on-6-(ethyl-2-yl)]-P²-(2′,3′-O-isopropylidene-6-chloronebularine-5-yl)methylenebis(phosphonate),

P¹-[7-hydroxy-5-methoxy-4-methylphthalan-1-on-6-(ethyl-2-yl)]-P²-(2 ′,3′-O-isopropylidene-6-methylthioguanosine-5-yl)methylenebis(phosphonate),

P¹-[7-hydroxy-5-methoxy-4-methylphthalan-1-on-6-(ethyl-2-yl)]-P²-(2′,3′-O-isopropylidene-8-azaadenosine-5-yl)methylenebis(phosphonate),

P¹-[7-hydroxy-5-methoxy-4-methylphthalan-1-on-6-(ethyl-2-yl)]-P²-(2′,3′-O-isopropylidene-8-azainosine-5-yl)methylenebis(phosphonate),

P¹-[7-hydroxy-5-methoxy-4-methylphthalan-1-on-6-(ethyl-2-yl)]-P²-(2′,3′-O-isopropylidene-8-azaguanosine-5-yl)methylenebis(phosphonate),

P¹-[7-hydroxy-5-methoxy-4-methylphthalan-1-on-6-(ethyl-2-yl)]-P²-(2 ′-O-acetyl-3′-deoxy-3′-fluoroinosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoroadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoroguanosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoronebularine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoroxanthosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-6-thioinosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-6-thioguanosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-O⁶-methylinosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-O⁶-methylguanosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-2-aminoadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-2-fluoroadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-2-chloroadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-2-bromoadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-6-chloronebularine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-6-methylthioguanosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-8-azaadenosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-8-azainosine-5-yl)methylenebis(phosphonate),

P¹-[7-hydroxy-5-methoxy-4-methylphthalan-1-on-6-(ethyl-2-yl)]-P²-(2′-O-acetyl-3′-deoxy-3′-fluoro-8-azaguanosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoroinosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoroadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoroguanosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoronebularine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-2-fluoroadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-6-thioinosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-6-thioguanosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-O⁶-methylinosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-O⁶-methylguanosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-2-aminoadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-2-fluoroadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-2-chloroadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-2-bromoadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-6-chloronebularine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-6-methylthioguanosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-8-azaadenosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-8-azainosine-5-yl)methylenebis(phosphonate),

P¹-[7-hydroxy-5-methoxy-4-methylphthalan-1-on-6-(ethyl-2-yl)]-P²-(3′-O-acetyl-2′-deoxy-2′-fluoro-8-azaguanosine-5-yl)methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)adenin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)hypoxanthin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)guanin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)purin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)xanthine-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-6-thiopurin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-6-thioguanine-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-6-methoxylpurin-5′-yl)methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-O⁶-methylguanine-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-2-aminoadenine-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-2-fluoroadenine-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-2-chloroadenin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-2-bromoadenin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-6-chloropurin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-6-methylthioguanin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-8-azaadenin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-8-azahypoxanthin-5′-yl]methylenebis(phosphonate),

P¹-[7-hydroxy-5-methoxy-4-methylphthalan-1-on-6-(ethyl-2-yl)]-P²-[9-(3-O-acetyl-2-deoxy-2-fluoro-β-D-arabinofuranosyl)-8-azaguanin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)adenin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)hypoxanthin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)guanin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)purin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)xanthine-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-6-thiopurin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-6-thioguanine-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-6-methoxylpurin-5′-yl)methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-06-methylguanine-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-2-aminoadenine-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-2-fluoroadenine-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-2-chloroadenin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-2-bromoadenin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-6-chloropurin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-6-methylthioguanin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-8-azaadenin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-8-azahypoxanthin-5′-yl]methylenebis(phosphonate),

P¹-[7-hydroxy-5-methoxy-4-methylphthalan-1-on-6-(ethyl-2-yl)]-P²-[9-(2-O-acetyl-3-deoxy-3-fluoro-β-D-xylofuranosyl)-8-azaguanin-5′-yl]methylenebis(phosphonate).

P¹-[7-hydroxy-5-methoxy-4-methylphthalan-1-on-6-(3-methylbut-2-ene-4-yl)]-P²-(2′,3′-O-isopropylidene-6-thioinosine-5-yl)methylenebis(phosphonate),

P¹-[7-hydroxy-5-methoxy-4-methylphthalan-1-on-6-(3-methylbut-2-ene-4-yl)]-P²(2′,3′-O-isopropylidene-6-thioguanosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-O⁶-methylinosine-5-yl)methylenebis(phosphonate),

P¹-[7-hydroxy-5-methoxy-4-methylphthalan-1-on-6-(3-methylbut-2-ene-4-yl)]-P² (2′,3′-O-isopropylidene-O⁶-methylguanosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-2-aminoadenosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-2-fluoroadenosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-2-chloroadenosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-2-bromoadenosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-6-chloronebularine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-6-methylthioguanosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-8-azaadenosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-8-azainosine-5-yl)methylenebis(phosphonate),

P¹-[7-hydroxy-5-methoxy-4-methylphthalan-1-on-6-(3-methylbut-2-ene-4-yl)]-P²-(2′,3′-O-isopropylidene-8-azaguanosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-6-thioinosine-5-yl)methylenebis(phosphonate),

P¹-[7-hydroxy-5-meth4oxy-4-methylphthalan-1-on-6-(3-methylhex-2-ene-6-yl)]-P²-(2′,3′-O-isopropylidene-6-thioguanosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-O⁶-methylinosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-O⁶-methylguanosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-2-aminoadenosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-2-fluoroadenosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-2-chloroadenosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-2-bromoadenosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-6-chloronebularine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-6-methylthioguanosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-8-azaadenosine-5-yl)methylenebis(phosphonate),

P¹-[7-hydroxy-5-methoxy-4-methylphthalan-1-on-6-(3-methylhex-2-ene-6-yl)]-P²-(2′,3′-O-isopropylidene-8-azainosine-5-yl)methylenebis(phosphonate), and

P¹-[7-hydroxy-5-methoxy-4-methylphthalan-1-on-6-(3-methylhex-2-ene-6-yl)]-P²-(2′,3′-O-isopropylidene-8-azaguanosine-5-yl)methylenebis(phosphonate).

The L-nucleoside-containing isomers of the above compounds are prepared by using the corresponding 2,3-O-protected L-nucleosides.

Biological Methods

Cell culture assays were used to determine the anti-Flaviviridae activity of unmodified or modified compounds of the formula (I) disclosed herein.

RNA Isolation and Quantitative RT-PCR Analysis

An effective process to quantify the viral load in a host, termed real-time polymerase chain reaction (“RT-PCR”) is provided. The process involves using a quenched fluorescent probe molecule which can be hybridized to viral DNA or RNA. Therefore, upon exonucleolytic degradation, a detectable fluorescent signal can be monitored. Therefore, the RT-PCR amplified DNA or RNA is detected in real time by monitoring the presence of fluorescence signals.

As one illustration of this method, in the case of BVDV in MDBK cells, in a first step, viral RNA is isolated from 140 μL of the cell culture supernatant by means of a commercially available column (Viral RNA extraction kit, QiaGen, CA). The viral RNA is then eluted from the column to yield a total volume of 60 μL, and subsequently amplified with a quantitative RT-PCR protocol using a suitable primer for the BVDV NADL strain. A quenched fluorescent probe molecule is hybridized to the BVDV DNA, which then undergoes exonucleolytic degradation resulting in a detectable fluorescent signal. Therefore, the RT-PCR amplified DNA was detected in real time by monitoring the presence of fluorescence signals. The TaqMan probe molecule (5′ 6-fam-AAATCCTCCTAACAAGCGGGTTCCAGG-tamara 3′ [Sequence ID No. 7] and primers (sense: 5′-AGCCTTCAGTTTCTTGCTGATGT-3′ [Sequence ID No. 8]; and antisense: 5′-TGTTGCGAAAGCACCAACAG-3′ [Sequence ID No. 9]) were designed with the aid of the Primer Express software (PE-Applied Biosystems) to be complementary to the BVDV NADL NS5B region. A total of 10 μL of RNA was analyzed in a 50 μL RT-PCR mixture. Reagents and conditions used in quantitative PCR were purchased from PE-Applied Biosystems. The standard curve that was created using the undiluted inoculum virus ranged from 6000 plaque forming units (PFU) to 0.6 PFU per RT-PCR mixture. A linear range of over 4-logs was routinely obtained.

Cell/Viral Materials

One of the best characterized members of the Pestivirus genus is BVDV. BVDV and HCV share at least three common features, which are the following: (1) they both undergo IRES-mediated translation; (2) NS4A cofactor is required by their NS3 serine protease; and (3) they undergo similar polyprotein processing within the non-structural region, especially at the NS5A and NS5B junction site.

The BVDV replication system was used for the discovery of anti-Flaviviridae compounds. The compounds described herein are active against Pestiviruses, Hepaciviruses and/or Flaviviruses.

Maldin-Darby bovine kidney (MDBK) cells were grown and maintained in a modified eagle medium (DMEM/F12; GibcoBRL), supplemented with 10% heat inactivated horse serum at 37° C. in a humidified, 5% CO₂, incubator.

Bovine viral diarrhea virus (BVDV), strain NADL, causes a cytopathogenic effect (CPE) after infection of these cells.

### Example 13

### Cytopathic Effect (CPE) Assay

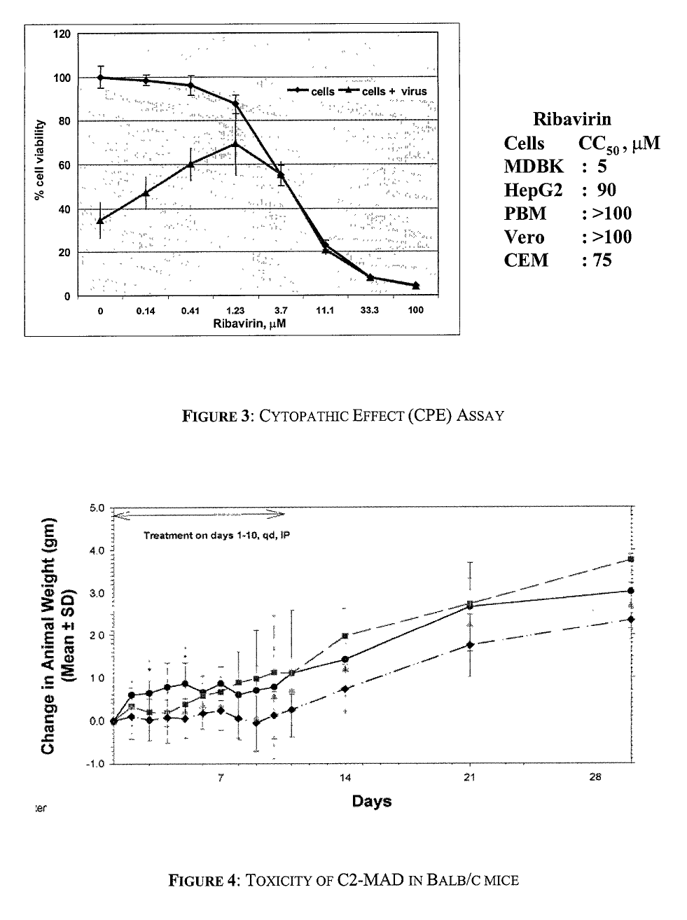

MDBK-cells, grown in DMEM/F12—10% horse serum (HS), were isolated using standard techniques using trypsin-EDTA. Cells were seeded in a 96-well plate at 5×10⁴ cells/well, with test compound (20 micromolar (μM) concentration) to give a total volume of 100 microliters (μL). After one hour, the media was removed and the cells were infected with cpBVDV (NADL) at a multiplicity of infection (MOI) of 0.02 or 0.002 in a total volume of 50 μL for 45 minutes. Thereafter, the virus was removed and the cells were washed twice with 100 μL of assay media. Finally, the infected cells were incubated in a total volume of 100 μL containing the test compound at 40 or 100 μM concentration. After 22 hours, the cell supernatant was collected by removing the cellular debris by low-speed centrifugation, and subsequently tested for the presence of virus in a quantitative manner. This assay proved problematic since MDBK cells are sensitive to the compounds of the present invention. The results of this assay using Ribavirin are illustrated in FIG. 3.

### Example 14

### Ttoxicity Testing of Candidate Anti-Flaviviridae Compounds

Cytotoxicity testing can be carried out according to standard methods. MDBK, PBM, HepG2, Huh7 and Vero cells were used. Briefly, cells are seeded in 96-well plates at various concentrations (dependent on cell type, duration of assay), typically at 5×10³ cells per well, in the presence of increasing concentrations of the test compound (0, 1, 3, 10, 33 and 100 μM). After a three day-incubation, cell viability and mitochondrial activity are measured by adding the MTS-dye (Promega), followed by a 3 hours incubation. Afterwards the plates containing the dye are read at 490 nm. Each assay was done in triplicate. Such methodologies are well described and available from the manufacturer (Promega).

In addition, the toxicity of C2-MAD was tested in Balb/c mice (six mice per group) at various concentrations (10, 30 and 60 mg/kg/day for ten days) as compared to water over a period of ten days. The results of this screening are illustrated in FIG. 4.

### Example 15

### The BVDV RT-PCR Quantification Standard Curve

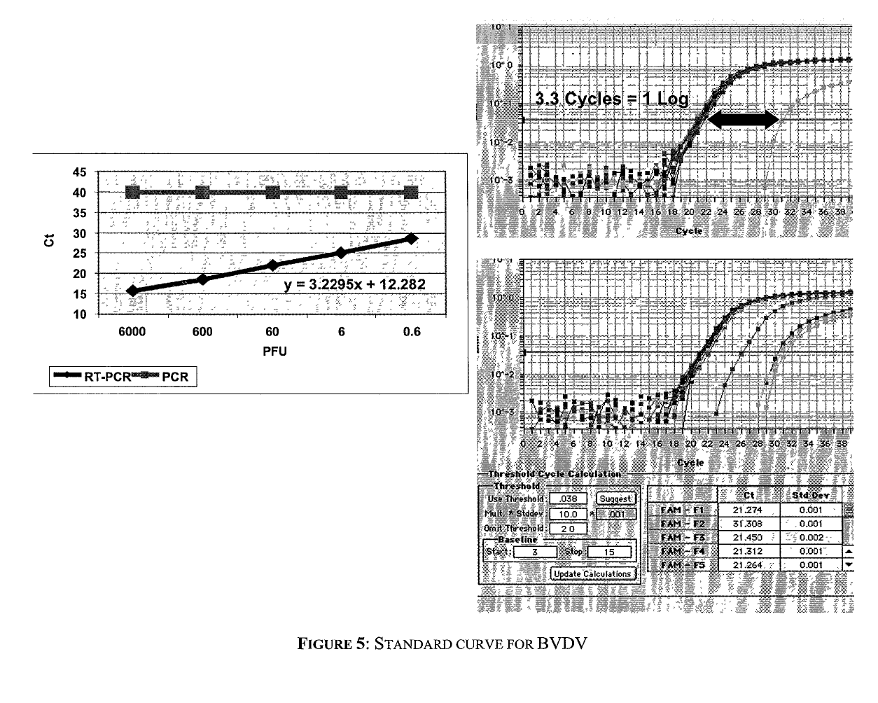

The standard BVDV virus stock contained 2×10⁶ PFU/ml, as determined by routine plaque assay (Mendez, E. et al. J. Virol. 1998, 72, 4737). Viral RNA was extracted from 140 μL of this inoculum material and eluted from a column using 60 μL of an elution buffer. This purified RNA material then was diluted stepwise from 10⁻¹ to 10⁻⁵. Using the real-time RT-PCR amplification technique, 10 μL of each dilution was tested. The results of this dilution series are plotted in FIG. 5, relating PFU to concentration of standard. From this experiment, it is clear that this technology allows for reliable quantification over 4-logs of virus (from 6000 to 0.6 PFU/input in amplification mix). The lower limit of detection in this experiment is 0.6 PFU or −0.22 log PFU. Therefore, the real-time RT-PCR quantification values of test samples below this detection limit were considered non-reliable.

### Example 16

### The BVDV Replication Cycle in MDBK Cells

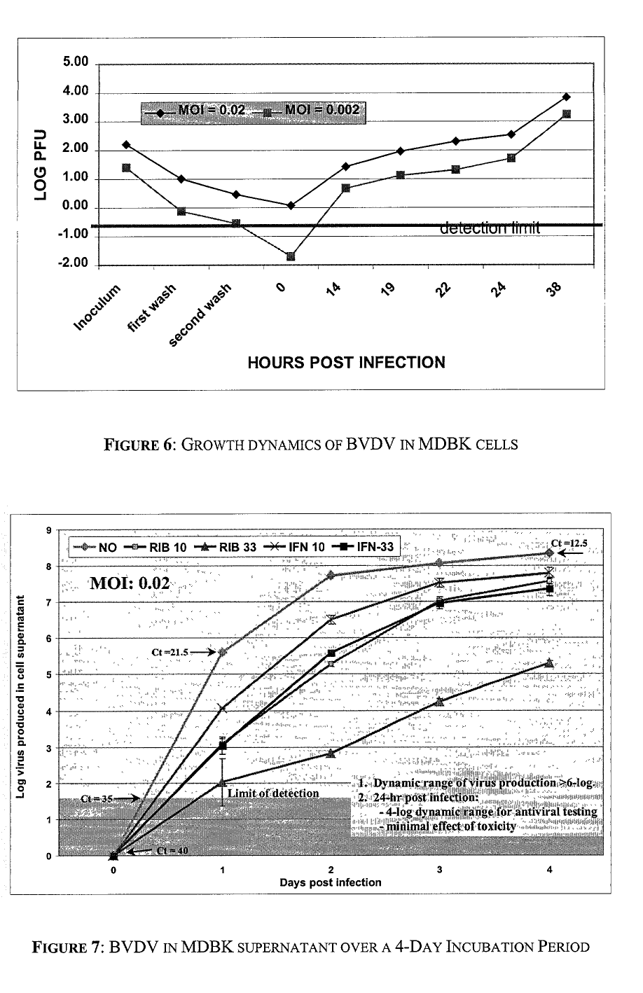

In order to measure the BVDV production in MDBK cells and to determine the optimal harvesting time over a certain period of time, cells were seeded at 5×10⁴ cells/well and infected either with MOI=0.02 or MOI=0.002. After infection, the inoculum was removed and the cells were washed twice with culture medium. At different time points, the cell supernatant was harvested; and, the amount of virus was measured and compared to the original inoculum and the cell wash, as shown in FIG. 6. At least 2 wash-steps were needed to remove the inoculum virus. The amount of virus produced 22 hours after infection approximately equals the amount of virus used to inoculate the cells. Based on these results, the time required for one replication cycle of BVDV in MDBK cells was 22 hours. Note that the detection level set in these experiments was based on the lower limit of detection as determined by the standard curve.

The BVDV production in MDBK cell over a four-day incubation period was also assessed relative to ribavirin (RIB) and interferon (IFN), as shown in FIG. 7.

### Example 17

### Evaluation of Candidate Antiviral Compounds Using RT-PCR

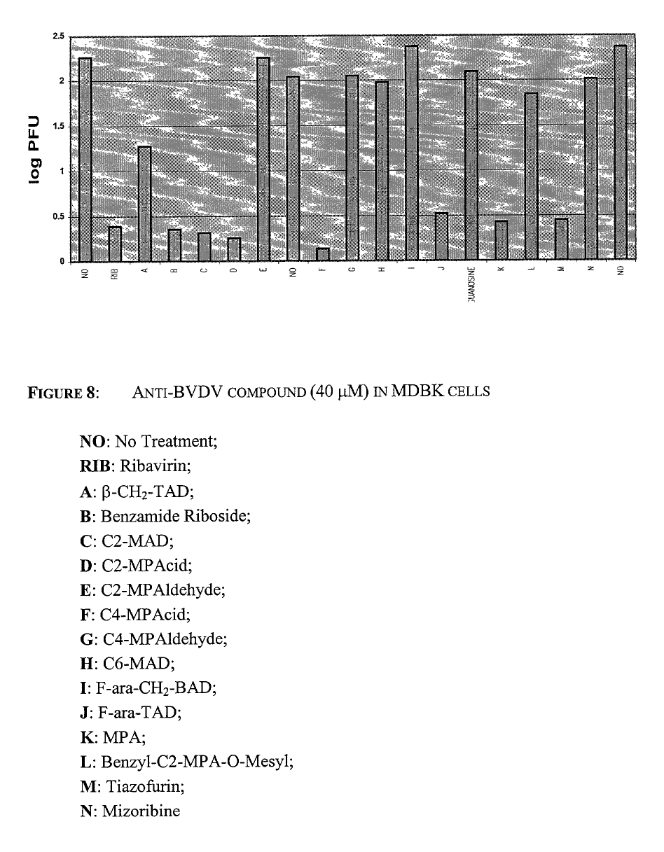

MDBK cells were seeded at 5×10⁴ cells/well, infected with BVDV with a MOI equal to 0.02 and grown for 22 hours in the presence of a test compound. Cells that were not treated with a test compound were considered a negative control, while ribavirin served as a positive control. Viral RNA was extracted and analyzed by real time RT-PCR. A typical experiment, shown in FIG. 4, demonstrates that the negative control and the compounds C2-MPAldehyde, C4-MPAlddehyde, C6-MAD, F-ara-CH₂-BAD, guanosine, benzyl-C2-MPA-O-mesyl and mizoribine produced comparable amounts of virus, effectively showing the test compounds as non-active. However, the cells treated with the positive control, ribavirin (RIB) or with mycophenolic acid or tiazofurin show an almost complete absence of viral RNA. Other active compounds are listed in Table 1 and/or shown in FIG. 8. RIB and these active compounds reduce viral production by approximately 2 log PFU, or 99%, in the 22 hour replication period. The exact potency of these compounds cannot be deduced from this experiment, since the detection limit in this experiment is set at −0.22 log PFU and only one cycle of viral replication occurs under the stated experimental conditions.

Potencies, or the effect concentration of compounds that inhibits virus production by 50% or 90% (EC₅₀ or EC₉₀ values, respectively), of anti-BVDV compounds were determined in a similar set of experiments, but over a broad range of test compound concentrations (0, 1, 3, 10, 33, 100 μM). The EC₉₀ value refers to the concentration necessary to obtain a 1-log reduction in viral production within a 22 hour period. Compounds that showed potent antiviral activity are listed in Table 1 and Table 2. Toxicity data were also obtained and are included, when available. Controls were Ribavirin and the polyoxymetalate HPA-023 in these experiments.

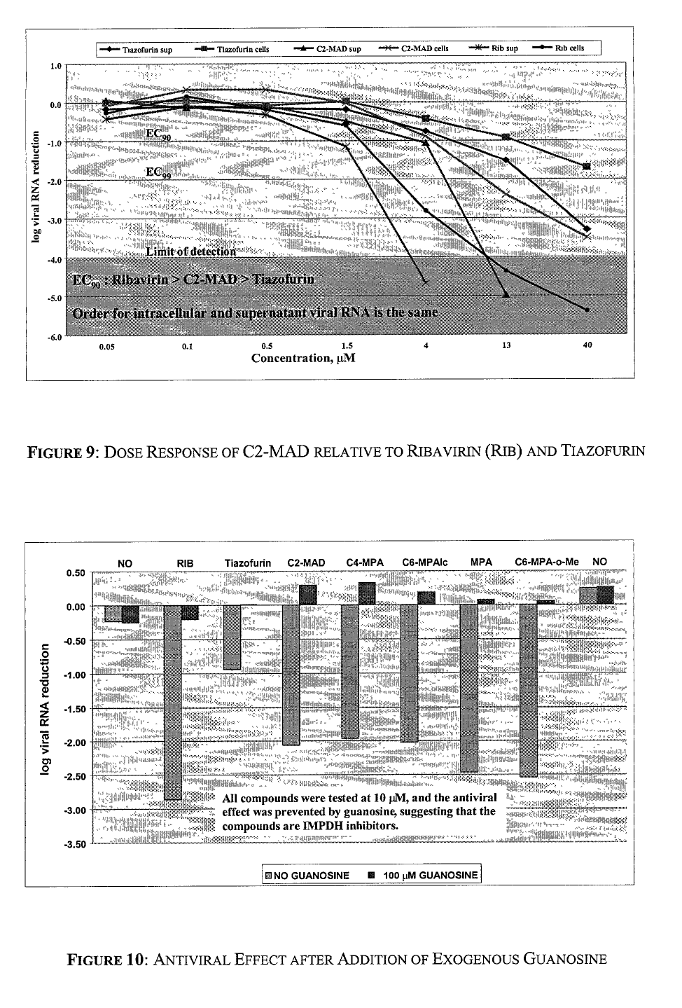

The dose-response for ribavirin (Rib) and Tiazofurin were compared to C2-MAD 24 hours post-infection, as shown in FIG. 9. The prevention of antiviral effect by exogenous addition of guanosine 24 hours post-infection was also measured to determine if the compounds were IMPDH inhibitors. As shown in FIG. 10, the IMPDH activity does not always correlate with anti-BVDV activity. For example, Mizoribine is a very potent human IMPDH inhibitor (Kᵢ=0.004 μM), but not active against BVDV (EC₉₀>100 μM), while C2-MAD is a potent IMPDH inhibitor (Kᵢ=0.3 μM), as well as a potent inhibitor of BVDV (EC₉₀=4 μM).

TABLE 1

| | ANTIVIRAL ACTIVITY | | CYTOTOXICITY^{b} | | | | |
|---|---|---|---|---|---|---|---|
| | BVDV real time RT-PCR^{a} | | MDBK | VERO | CEM | PBMC | HEPG2 |
| | max log reduction^{c} | EC₅₀/EC₉₀ | 3 days | 3 days | 7 days | 7 days | 3 days |
| COMPOUND | Viral Load | (μM) | CC₅₀ (μM) | CC₅₀ (μM) | CC₅₀ (μM) | CC₅₀ (μM) | CC₅₀ (μM) |
| Ribavirin | >2 (40 μM) | 0.7/5 | 4 | >100 | 75 | >100 | 80 |
| HPA-023 | >2 (40 μM) | /8 | 45 | 2.35 | >100 | 100 | 20 |
| Mycophenolic Acid (MPA) | >2 (40 μM) | /1 | 2 | | 0.05 | 75.4 | |
| Tiazofurin | >2 (40 μM) | /30 | 25 | | 73.4 | 79.5 | >100 |
| Benzamide Riboside | >2 (40 μM) | /3 | 4 | | | | 8 |
| C2-MPAlcohol | 1.96 (40 μM) | /40 | 7 | | 91 | >100 | |
| C4-MPAlcohol | 2.08 (40 μM) | /6 | 8 | | 24.2 | 77.2 | |
| F-ara-CH₂-TAD | 1.69 (40 μM) | /30 | 80 | | 56.7 | 83.7 | |
| β-CH₂-TAD | 0.94 (40 μM) | / | 70 | | 47.8 | >100 | |
| C2-MAD | 1.9 (40 μM) | /6 | 50 | | 17.7 | 45.9 | |
| MPA-O-Me | 3.32 (100 μM) | | | | | | |
| C6-MPAlcohol | 3.22 (100 μM) | | | | | | |
| Benzyl-C2-MPA-O-Mesyl | 0.53 (100 μM) | | | | | | |
| C2-Myco-Phenetene | 0.97 (100 μM) | | | | | | |
| C2-MPAlcohol-O-Mesyl) | 3.41 (100 μM) | | | | | | |
| Mycophenetane | 2.74 (100 μM) | | | | | | |
| C4-MPAcid | 3.01 (100 μM) | | | | | | |
| C2-MPAlc-7O-benzyl | 1.13 (100 μM) | | | | | | |

^{a}determined after 22 hours.
^{b}cytotoxicity determined using either a cell-growth curve, or a colorometric assay using MTT or MTS as described by the manufacturer (Promega).
^{c}max log reduction tested at indicated concentration (40 μM or 100 μM).

TABLE 2

| | | MDBK | MDBK | Antiviral | Selectivity Index |
|---|---|---|---|---|---|
| | IMPDH | (3 Days) | (24 hrs) | (24 hrs) | (24 hrs/24 hrs) |
| ID | Kᵢ (μM) | CC₅₀ (μM) | CC₅₀ (μM) | EC₉₀ (μM) | CC₅₀/EC₉₀ |
| D-Ribavirin | 0.2 | 5 | >100 | 2 | 5 |
| L-Ribavirin | | >100 | >100 | >100 | 1 |
| Mizoribine | 0.004 | >100 | >100 | >100 | 1 |
| Tiazofurin | 0.11 | 25 | >100 | 7 | 14 |
| Benzamide Riboside | 0.66 | 3 | 13 | 3 | 4 |
| C6-MPAlc | 0.5 | 5 | 60 | 2 | 30 |
| MPA | 0.035 | 2 | >100 | <1 | >100 |
| C6-MPA-O-Me | 0.5 | 0.4 | >100 | <1 | >100 |
| C4-MPAlc | | 8 | >100 | 6 | 17 |
| C4-MPAld | | >100 | >100 | >100 | 1 |
| C4-MPAc | | 39 | >100 | 33 | 3 |
| C2-MPAlc | | 7 | >100 | 40 | 4 |
| C2-MPAld | | >100 | >100 | >100 | 1 |
| C2-MPAc | | >100 | >100 | >100 | 1 |
| F-ara-TAD | 2.6 | 80 | >100 | 30 | 3 |
| CH₂-TAD | 0.11 | 70 | >100 | >100 | 1 |
| F-ara-CH₂-TAD | | >100 | >100 | | |
| F-ara-CH₂-BAD | 175 | >100 | >100 | >100 | 1 |
| C6-MAD | 0.3 | >100 | >100 | >100 | 1 |
| C2-MAD | 0.3 | 9 | >100 | 4 | 25 |

HepG2 cells: CC₅₀ > 50 μM for all compounds, except Benzamide riboside (CC₅₀ = 8 μM)
PBM cells: CC₅₀ > 50 μM for all compounds, except Benzamide riboside, C6-MPAlc and C6-MPA-O-Me(CC₅₀ < 17 μM)

The invention has been described with reference to various specific and preferred embodiments and techniques. However, it should be understood that many variations and modifications will be obvious to those skilled in the art from the foregoing detailed description of the invention and may be made while remaining within the spirit and scope of the invention.

We claim:

1. A compound of the formula (I): 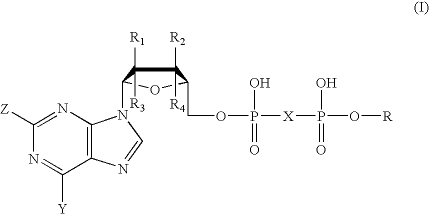
or its pharmaceutically acceptable salt thereof; wherein
R is 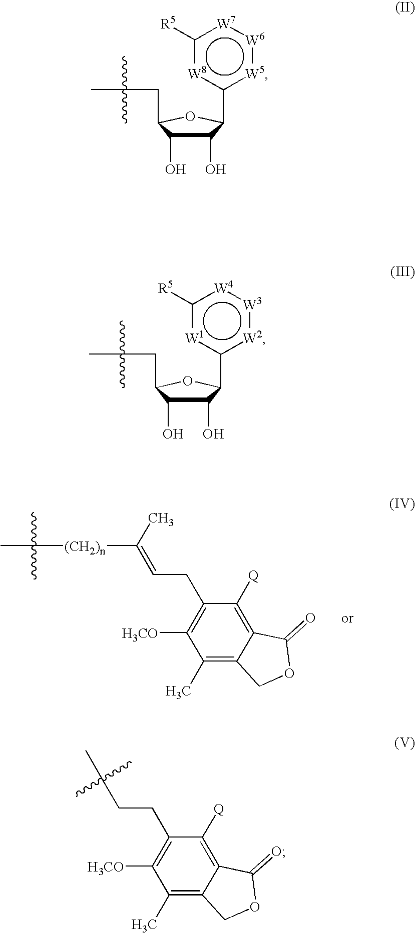
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD).

2. The compound of claim 1, wherein the compound of formula (I) is selected from the group consisting of the following: 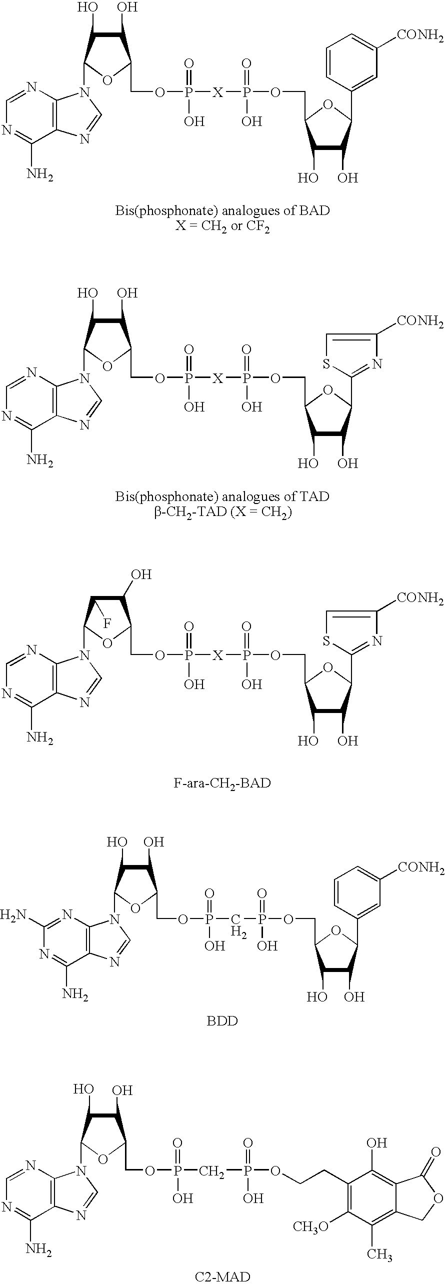 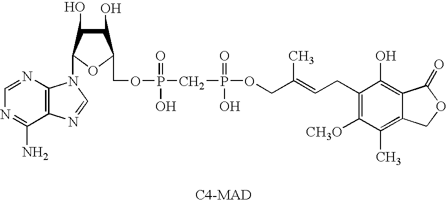
or its pharmaceutically acceptable salt thereof, wherein X is oxygen, sulfur, methylene, monofluoromethylene or difluoromethylene.

3. A compound of the formula: 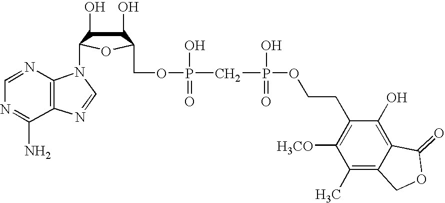
or its pharmaceutically acceptable salt thereof.

4. A compound selected from the group consisting of the following: 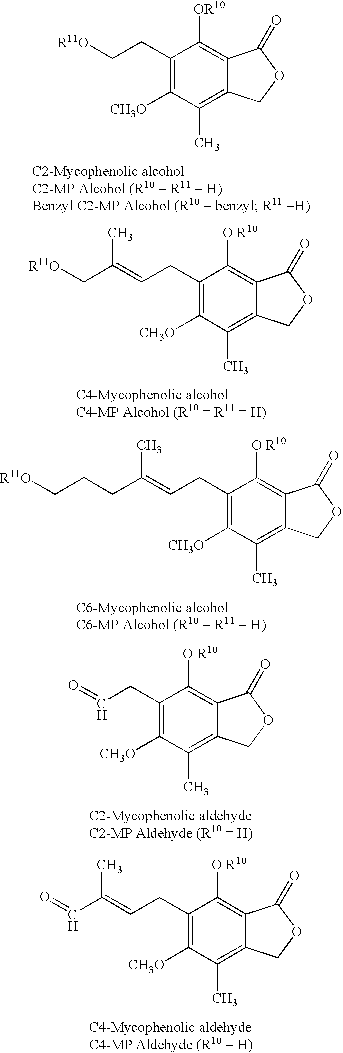 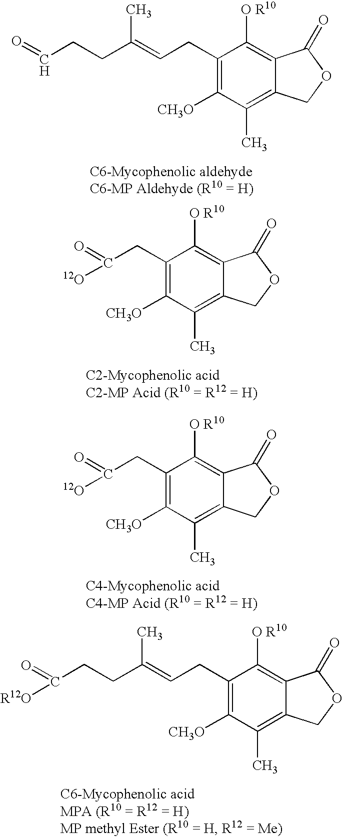
or its pharmaceutically acceptable salt thereof; wherein
each R¹⁰ and R¹¹ is independently hydrogen, alkyl, acyl, benzyl or methoxymethyl (MOM) group, and each R¹² is independently hydrogen, alkyl or aryl.

5. A compound selected from the group consisting of the following: 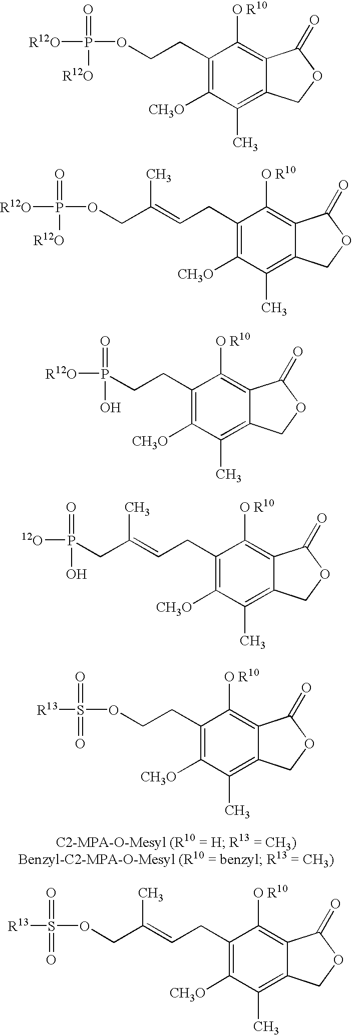
or its pharmaceutically acceptable salt thereof, wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl.

6. A compound selected from the group consisting of the following: 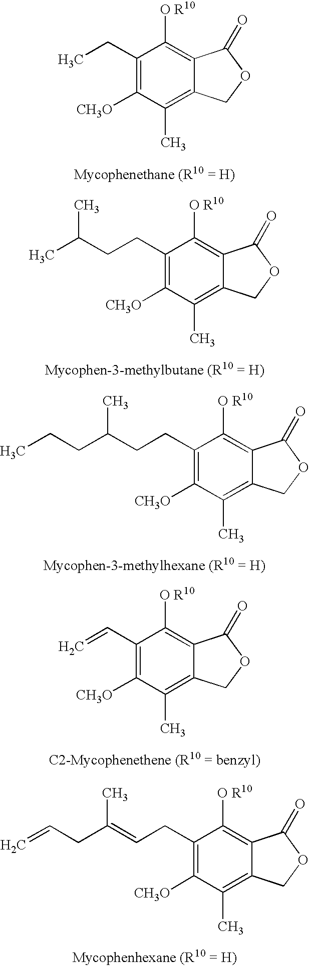
or its pharmaceutically acceptable salt thereof; wherein
each R¹⁰ is independently hydrogen, alkyl, acyl, benzyl or methoxymethyl (MOM) group.

7. A compound selected from the group consisting of the following: 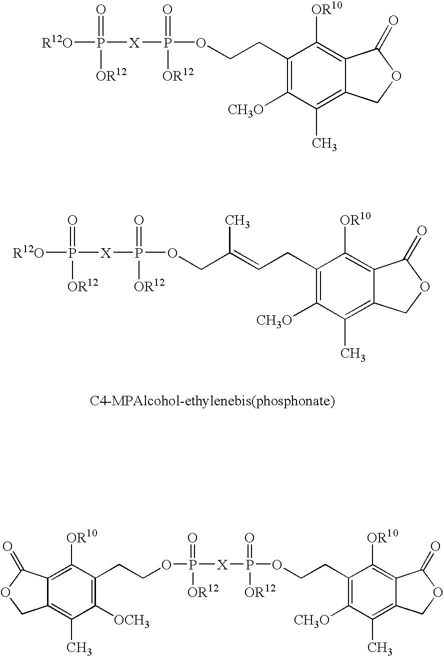
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl.

8. A pharmaceutical composition for the treatment or prophylaxis of a Flaviviridae infection in a host, comprising an effective amount of a compound of the formula (I): 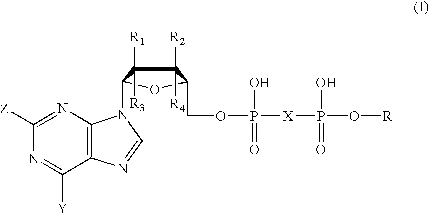
or its pharmaceutically acceptable salt thereof; wherein 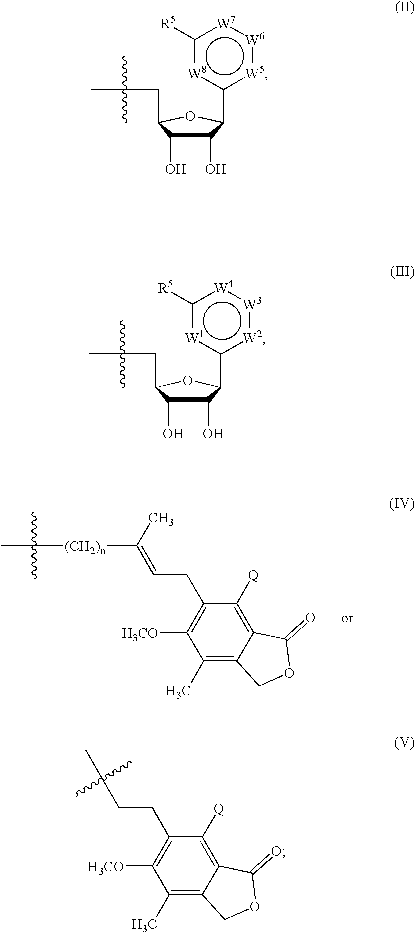
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
in a pharmaceutically acceptable carrier or diluent.

9. The pharmaceutical composition of claim 8, wherein the compound of formula (I) is selected from the group consisting of the following: 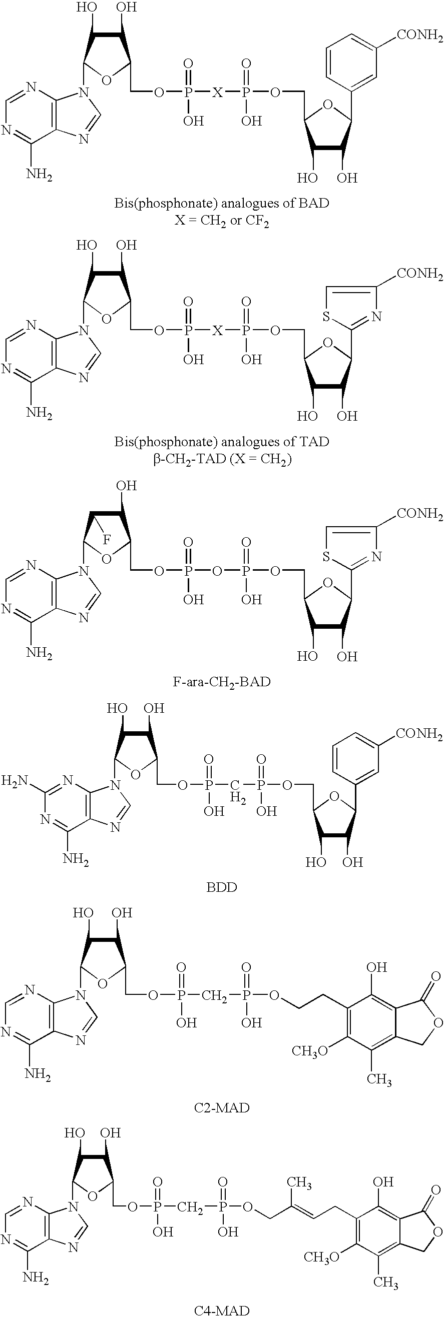
or its pharmaceutically acceptable salt thereof, wherein X is oxygen, sulfur, methylene, monofluoromethylene or difluoromethylene.

10. A pharmaceutical composition for the treatment or prophylaxis of a Flaviviridae infection in a host, comprising an effective amount of a compound of the formula: 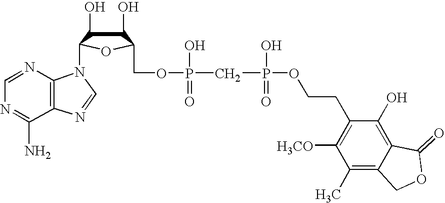
or its pharmaceutically acceptable salt thereof, in a pharmaceutically acceptable carrier or diluent.

11. A pharmaceutical composition for the treatment or prophylaxis of a Flaviviridae infection in a host, comprising an effective amount of a compound selected from the group consisting of the following: 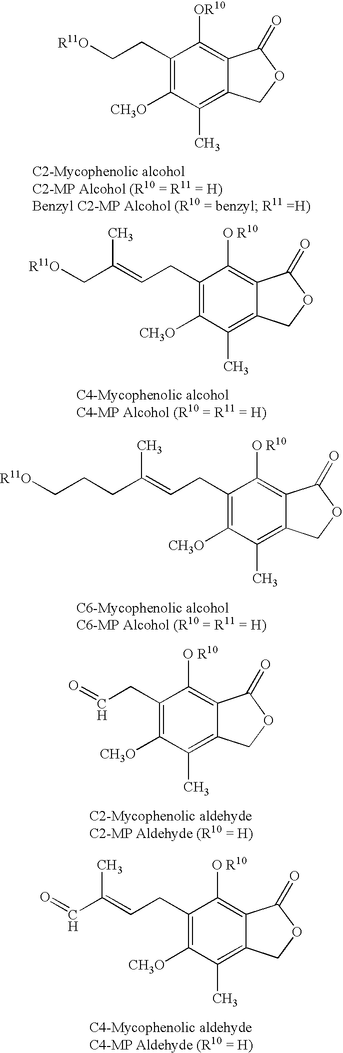 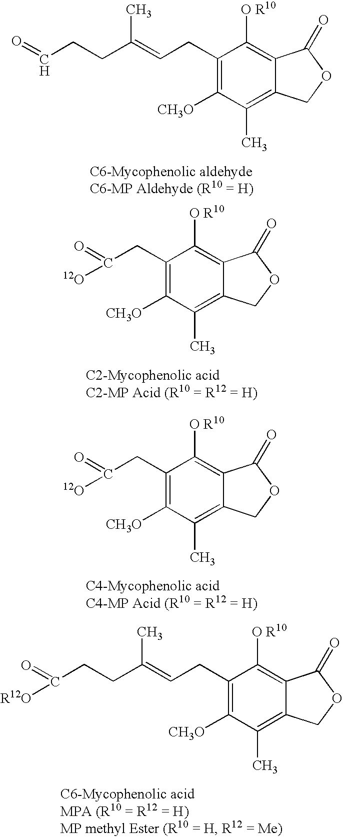
or its pharmaceutically acceptable salt thereof, wherein
each R¹⁰ and R¹¹ is independently hydrogen, alkyl, acyl, benzyl or methoxymethyl (MOM) group, and each R¹² is independently hydrogen, alkyl or aryl;
in a pharmaceutically acceptable carrier or diluent.

12. A pharmaceutical composition for the treatment or prophylaxis of a Flaviviridae infection in a host, comprising an effective amount of a compound selected from the group consisting of the following: 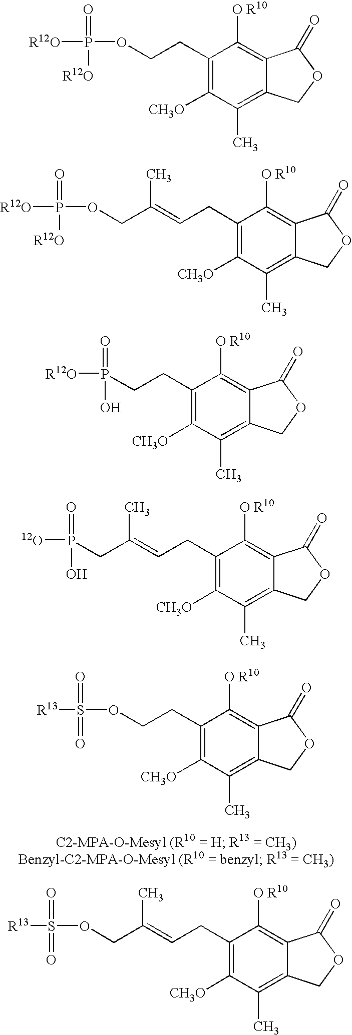
or its pharmaceutically acceptable salt thereof, wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
in a pharmaceutically acceptable carrier or diluent.

13. A pharmaceutical composition for the treatment or prophylaxis of a Flaviviridae infection in a host, comprising an effective amount of a compound selected from the group consisting of the following: 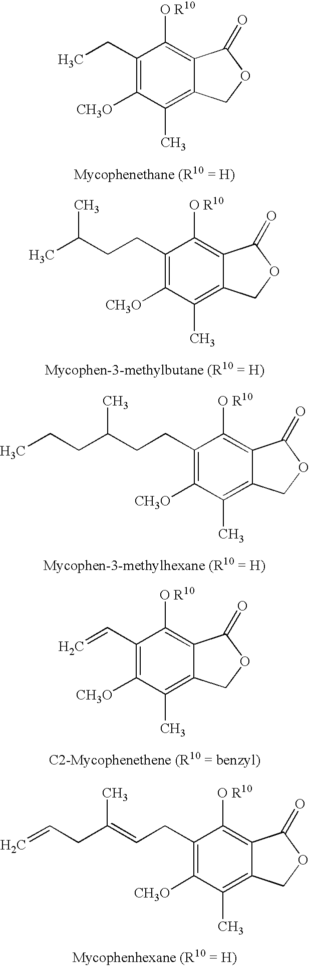
or its pharmaceutically acceptable salt thereof; wherein
each R¹⁰ is independently hydrogen, alkyl, acyl, benzyl or methoxymethyl (MOM) group;
in a pharmaceutically acceptable carrier or diluent.

14. A pharmaceutical composition for the treatment or prophylaxis of a Flaviviridae infection in a host, comprising an effective amount of a compound selected from the group consisting of the following: 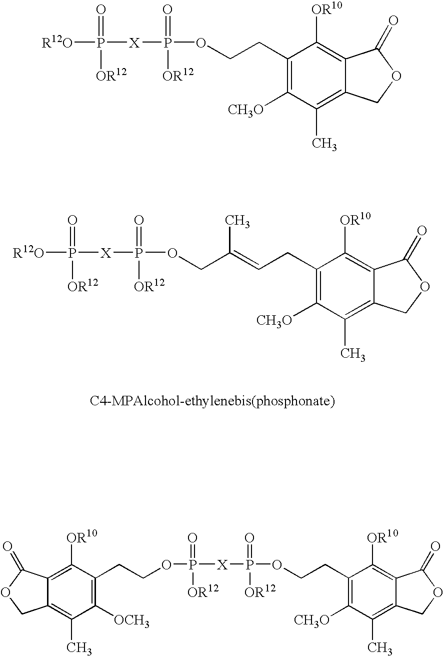
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
in a pharmaceutically acceptable carrier or diluent.

15. A pharmaceutical composition for the treatment or prophylaxis of a Flaviviridae infection in a host, comprising an effective amount of a compound of the formula (I): 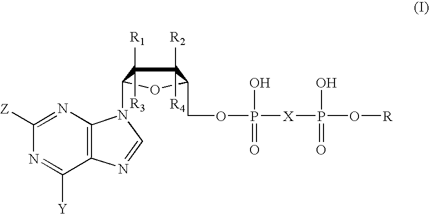
or its pharmaceutically acceptable salt thereof; wherein
R is 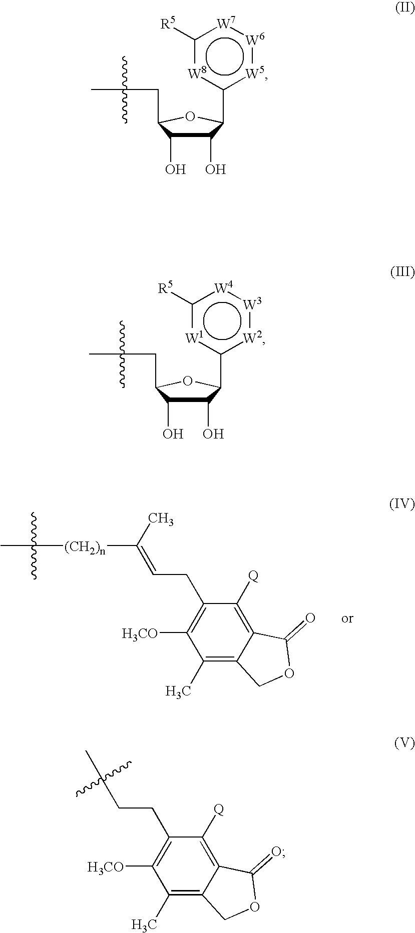
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
in combination with one or more other antivirally effective agent, optionally in a pharmaceutically acceptable carrier or diluent.

16. The pharmaceutical composition of claim 15, wherein the compound of formula (I) is selected from the group consisting of the following: 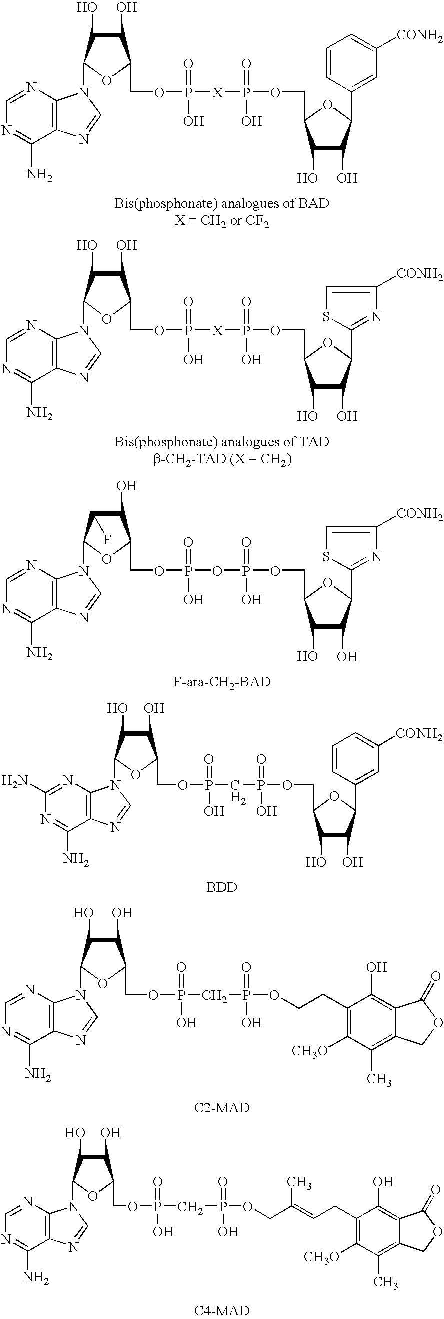
or its pharmaceutically acceptable salt thereof, wherein X is oxygen, sulfur, methylene, monofluoromethylene or difluoromethylene.

17. A pharmaceutical composition for the treatment or prophylaxis of a Flaviviridae infection in a host, comprising an effective amount of a compound of the formula: 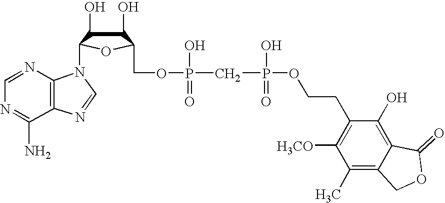
or its pharmaceutically acceptable salt thereof, in combination with one or more other antivirally effective agent, optionally in a pharmaceutically acceptable carrier or diluent.

18. A pharmaceutical composition for the treatment or prophylaxis of a Flaviviridae infection in a host, comprising an effective amount of a compound selected from the group consisting of the following: 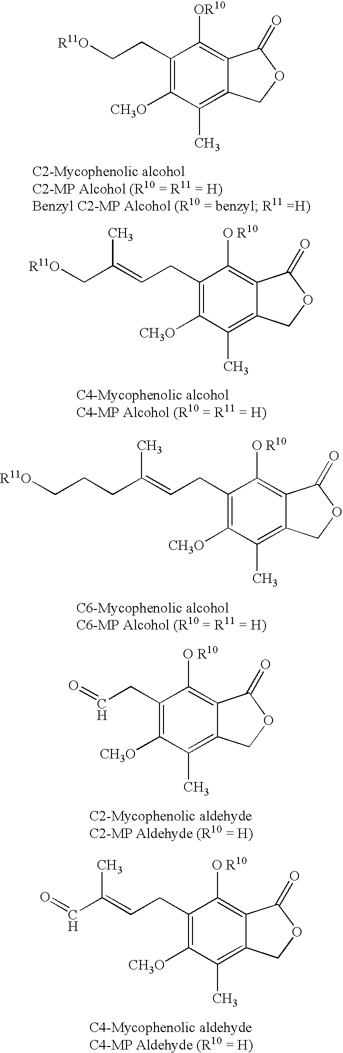 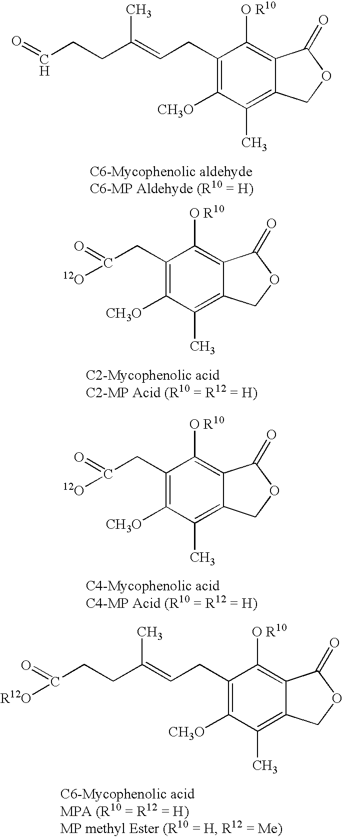
or its pharmaceutically acceptable salt thereof; wherein
each R¹⁰ and R¹¹ is independently hydrogen, alkyl, acyl, benzyl or methoxymethyl (MOM) group, and each R¹² is independently hydrogen, alkyl or aryl;
in combination with one or more other antivirally effective agent, optionally in a pharmaceutically acceptable carrier or diluent.

19. A pharmaceutical composition for the treatment or prophylaxis of a Flaviviridae infection in a host, comprising an effective amount of a compound selected from the group consisting of the following: 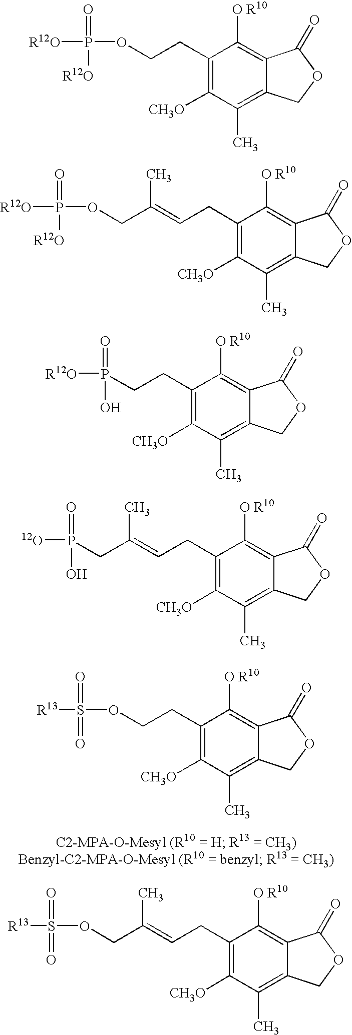
or its pharmaceutically acceptable salt thereof; wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
in combination with one or more other antivirally effective agent, optionally in a pharmaceutically acceptable carrier or diluent.

20. A pharmaceutical composition for the treatment or prophylaxis of a Flaviviridae infection in a host, comprising an effective amount of a compound selected from the group consisting of the following: 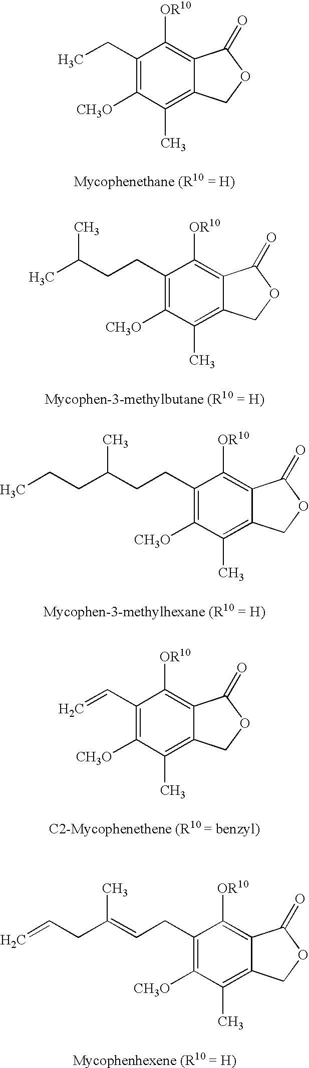
or its pharmaceutically acceptable salt thereof; wherein
each R¹⁰ is independently hydrogen, alkyl, acyl, benzyl or methoxymethyl (MOM) group;
in combination with one or more other antivirally effective agent, optionally in a pharmaceutically acceptable carrier or diluent.

21. A pharmaceutical composition for the treatment or prophylaxis of a Flaviviridae infection a host, comprising an effective amount of a compound selected from the group consisting of the following: 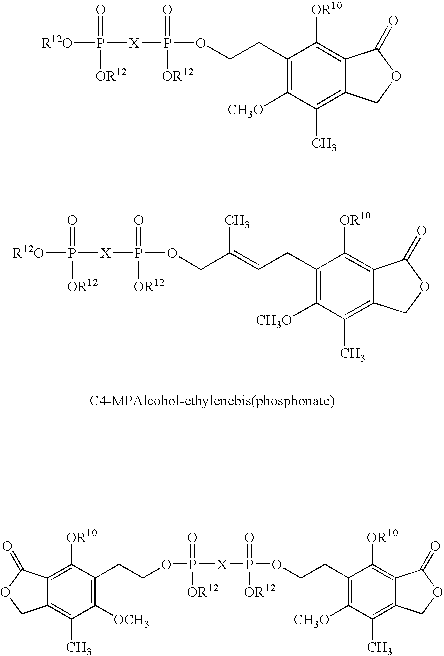
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
in combination with one or more other antivirally effective agent, optionally in a pharmaceutically acceptable carrier or diluent.

22. A method for the treatment or prophylaxis of a Flaviviridae infection in a host, comprising administering an effective amount of a compound of the formula (I): 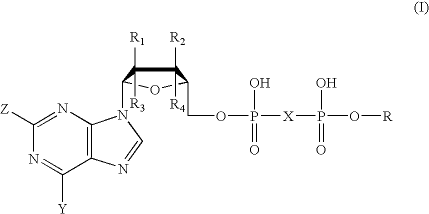
or its pharmaceutically acceptable salt thereof; wherein
R is 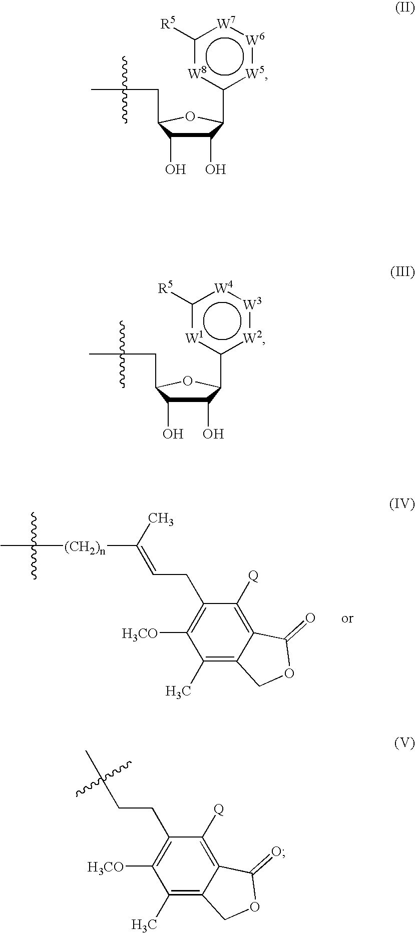
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
optionally in a pharmaceutically acceptable carrier or diluent.

23. The method of claim 22, wherein the compound of formula (I) is selected from the group consisting of the following: 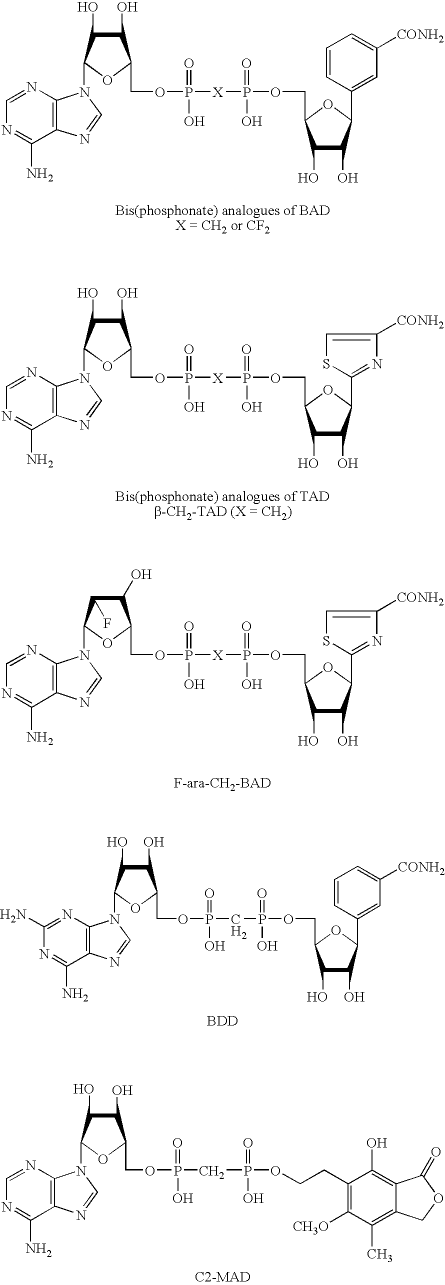 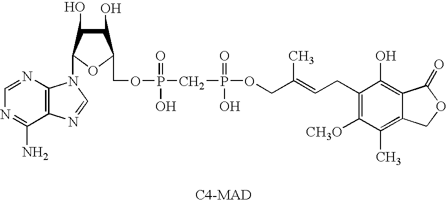
or its pharmaceutically acceptable salt thereof, wherein X is oxygen, sulfur, methylene, monofluoromethylene or difluoromethylene.

24. A method for the treatment or prophylaxis of a Flaviviridae infection in a host, comprising administering an effective amount of a compound of the formula: 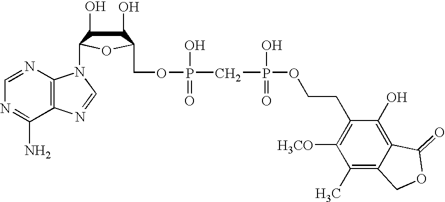
or its pharmaceutically acceptable salt thereof, optionally in a pharmaceutically acceptable carrier or diluent.

25. A method for the treatment or prophylaxis of a Flaviviridae infection in a host, comprising administering an effective amount of a compound selected from the group consisting of the following: 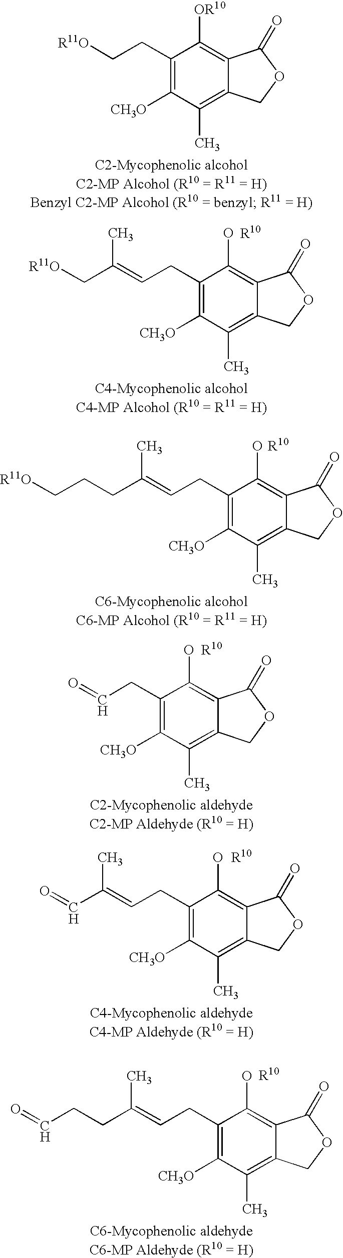 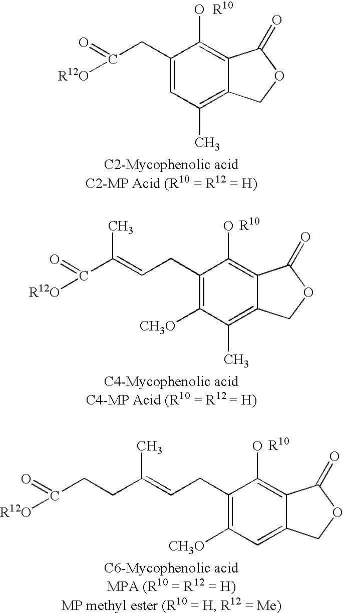
or its pharmaceutically acceptable salt thereof; wherein
each R¹⁰ and R¹¹ is independently hydrogen, alkyl, acyl, benzyl or methoxymethyl (MOM) group, and each R¹² is independently hydrogen, alkyl or aryl;
optionally in a pharmaceutically acceptable carrier or diluent.

26. A method for the treatment or prophylaxis of a Flaviviridae infection in a host, comprising administering an effective amount of a compound selected from the group consisting of the following: 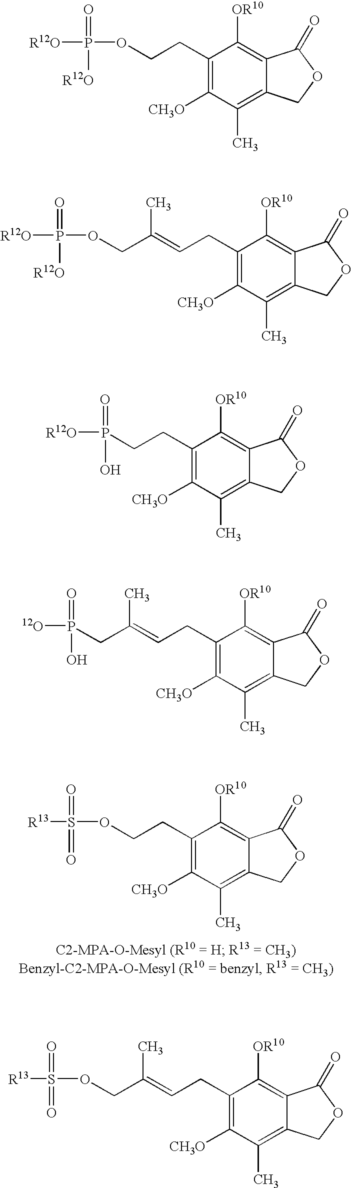
or its pharmaceutically acceptable salt thereof; wherein
each R¹⁰ is independently hydrogen, alkyl, acyl, benzyl or methoxymethyl (MOM) group;
each R² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
optionally in a pharmaceutically acceptable carrier or diluent.

27. A method for the treatment or prophylaxis of a Flaviviridae infection in a host, comprising administering an effective amount of a compound selected from the group consisting of the following: 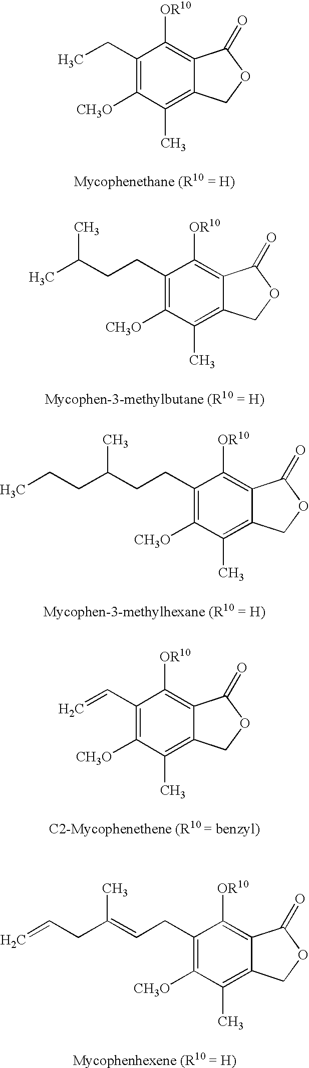
or its pharmaceutically acceptable salt thereof; wherein
each R¹⁰ is independently hydrogen, alkyl, acyl, benzyl or methoxymethyl (MOM) group;
optionally in a pharmaceutically acceptable carrier or diluent.

28. A method for the treatment or prophylaxis of a Flaviviridae infection in a host, comprising administering an effective amount of a compound selected from the group consisting of the following: 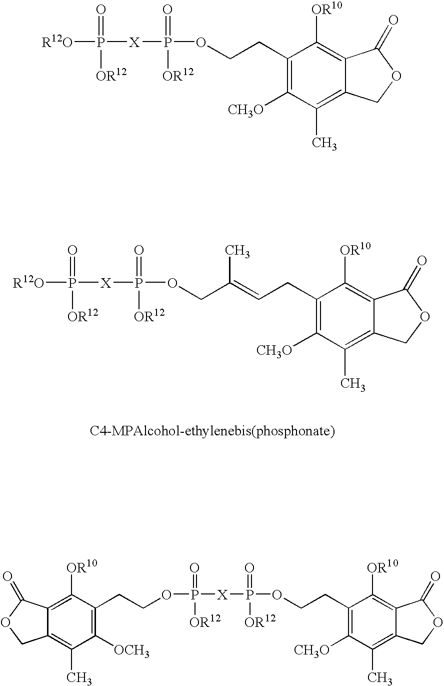
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
optionally in a pharmaceutically acceptable carrier or diluent.

29. A method for the treatment or prophylaxis of a Flaviviridae infection in a host, comprising administering an effective amount of a compound of the formula (I): 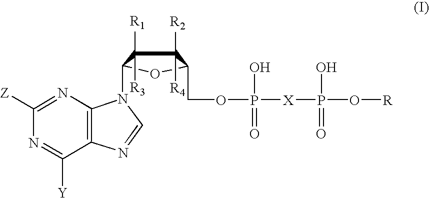
or its pharmaceutically acceptable salt thereof; wherein
R is 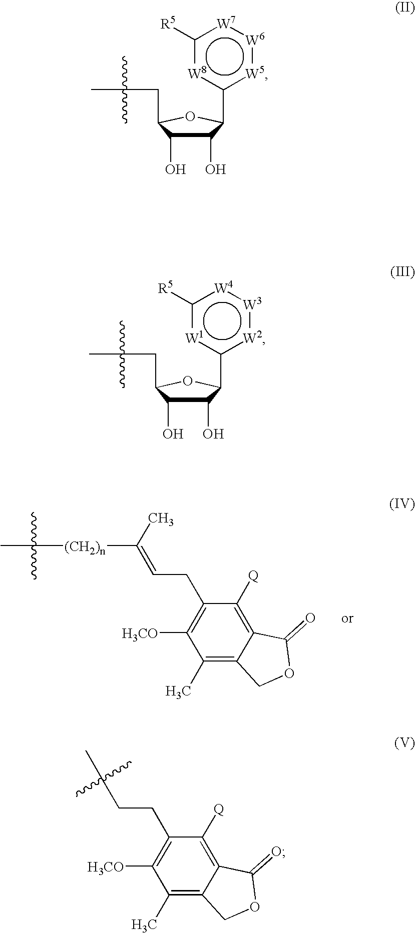
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
in combination or alternation with one or more other antivirally effective agent, optionally in a pharmaceutically acceptable carrier or diluent.

30. The method of claim 29, wherein the compound of formula (I) is selected from the group consisting of the following: 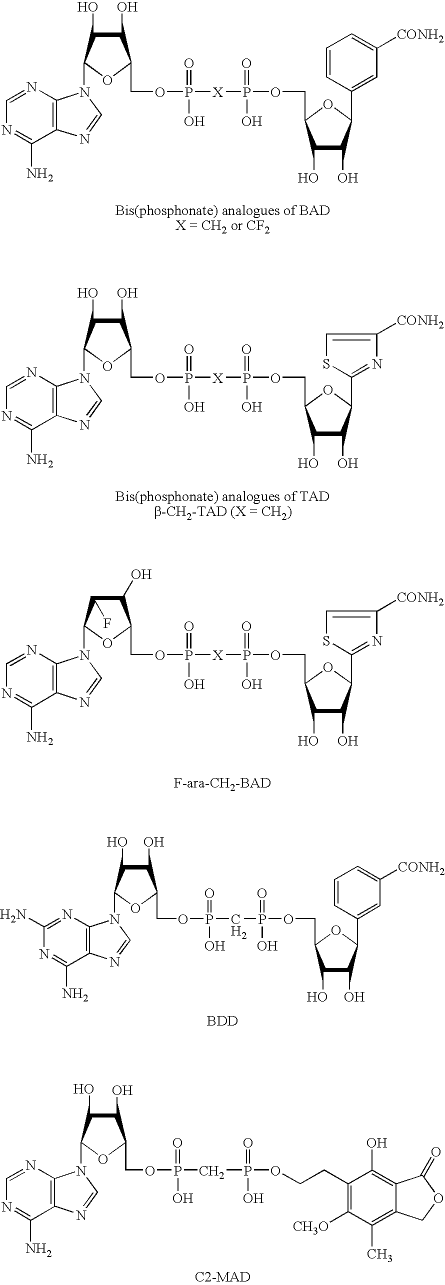 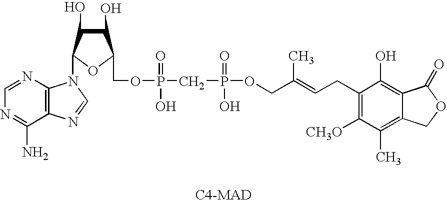
or its pharmaceutically acceptable salt thereof, wherein X is oxygen, sulfur, methylene, monofluoromethylene or difluoromethylene.

31. A method for the treatment or prophylaxis of a Flaviviridae infection in a host, comprising administering an effective amount of a compound of the formula: 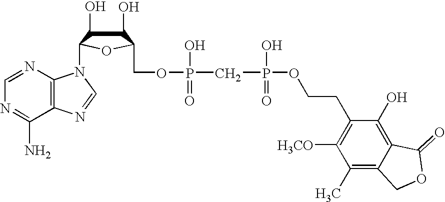
or its pharmaceutically acceptable salt thereof, in combination or alternation with one or more other antivirally effective agent, optionally in a pharmaceutically acceptable carrier or diluent.

32. A method for the treatment or prophylaxis of a Flaviviridae infection in a host, comprising administering an effective amount of a compound selected from the group consisting of the following: 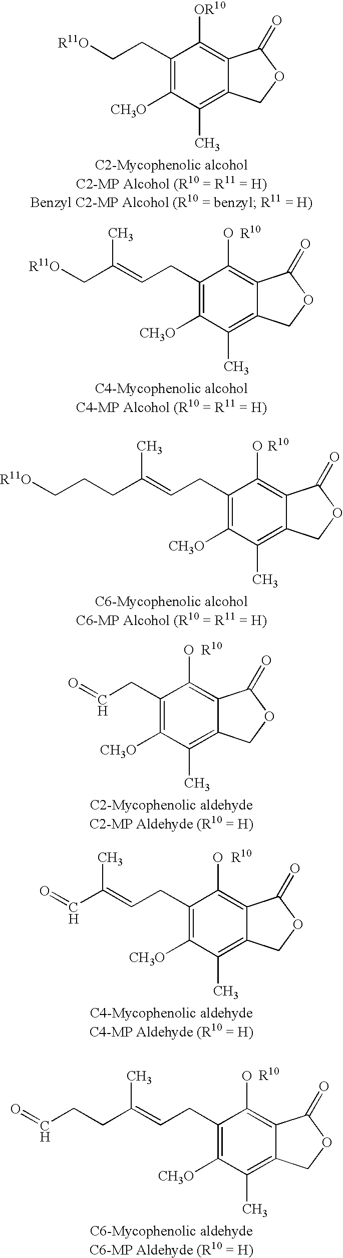 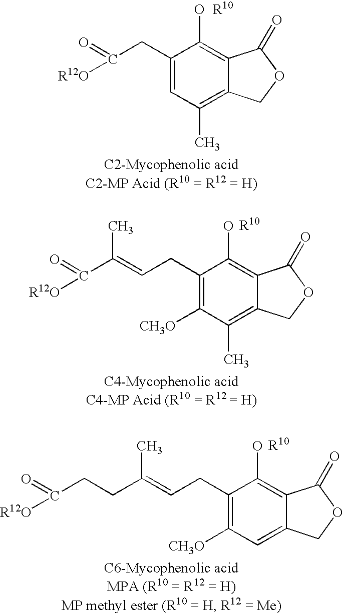
or its pharmaceutically acceptable salt thereof, wherein
each R¹⁰ and R¹¹ is independently hydrogen, alkyl, acyl, benzyl or methoxymethyl (MOM) group, and each R¹² is independently hydrogen, alkyl or aryl;
in combination or alternation with one or more other antivirally effective agent, optionally in a pharmaceutically acceptable carrier or diluent.

33. A method for the treatment or prophylaxis of a Flaviviridae infection in a host, comprising administering an effective amount of a compound selected from the group consisting of the following: 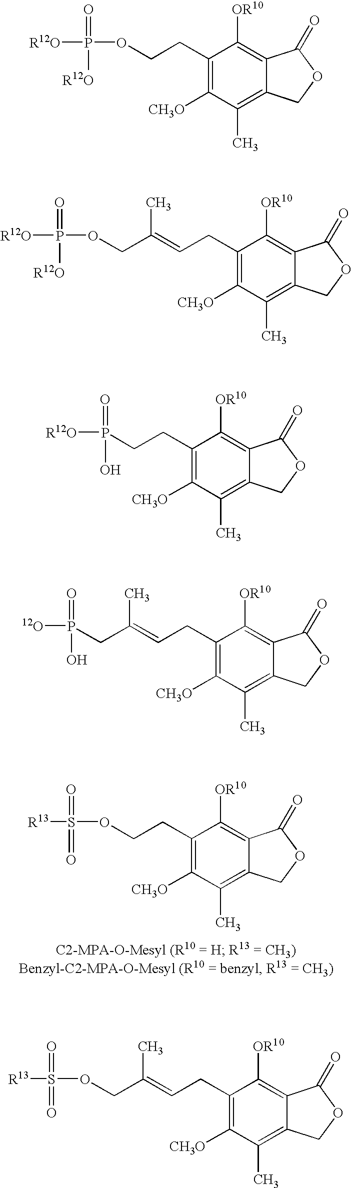
or its pharmaceutically acceptable salt thereof; wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
in combination or alternation with one or more other antivirally effective agent, optionally in a pharmaceutically acceptable carrier or diluent.

34. A method for the treatment or prophylaxis of a Flaviviridae infection in a host, comprising administering an effective amount of a compound selected from the group consisting of the following: 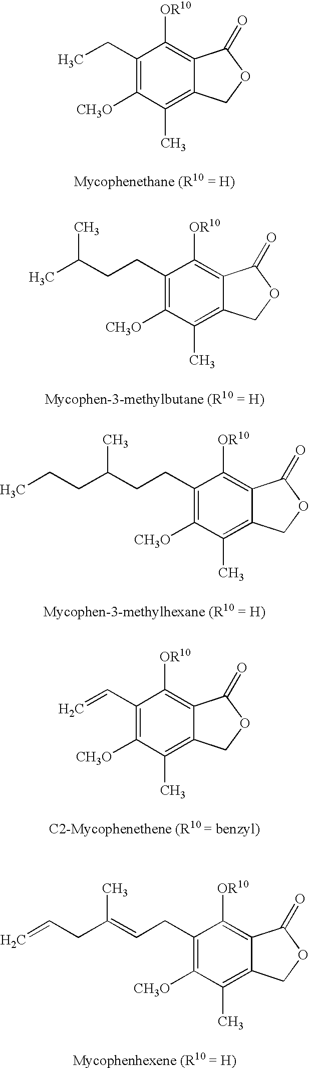
or its pharmaceutically acceptable salt thereof; wherein
each R¹⁰ is independently hydrogen, alkyl, acyl, benzyl or methoxymethyl (MOM) group;
in combination or alternation with one or more other antivirally effective agent, optionally in a pharmaceutically acceptable carrier or diluent.

35. A method for the treatment or prophylaxis of a Flaviviridae infection in a host, comprising administering an effective amount of a compound selected from the group consisting of the following: 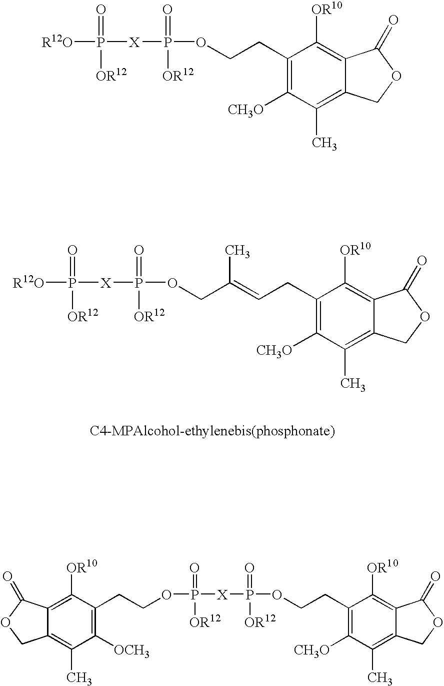
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
in combination or alternation with one or more other antivirally effective agent, optionally in a pharmaceutically acceptable carrier or diluent.

36. A method for the treatment or prophylaxis of abnormal cellular proliferation in a host, comprising administering an effective amount of a compound of the formula (I): 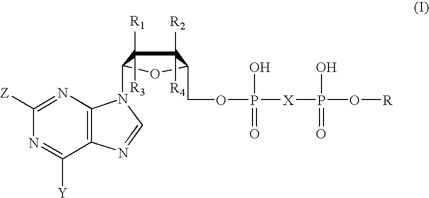
or its pharmaceutically acceptable salt thereof; wherein
R is 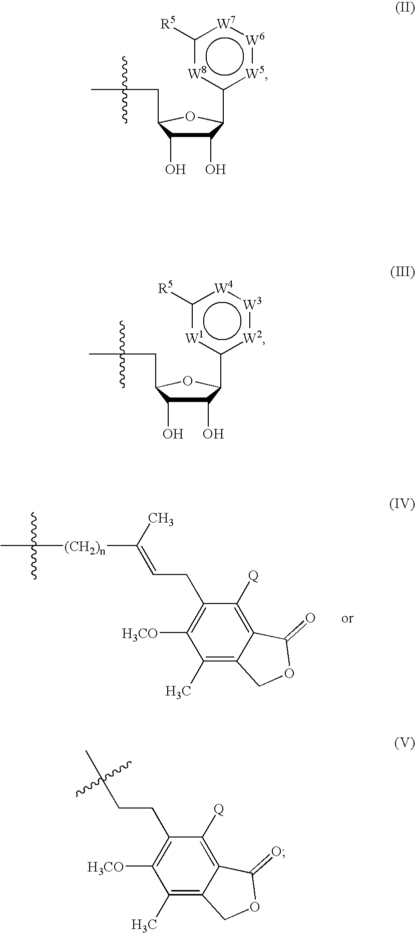
X is oxygen, sulfur, methylene, monofluoromethylene or difluoromethylene;
Y is hydrogen, halogen (F, Cl , Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
optionally in a pharmaceutically acceptable carrier or diluent.

37. The method of claim 22, wherein the compound of formula (I) is selected from the group consisting of the following: 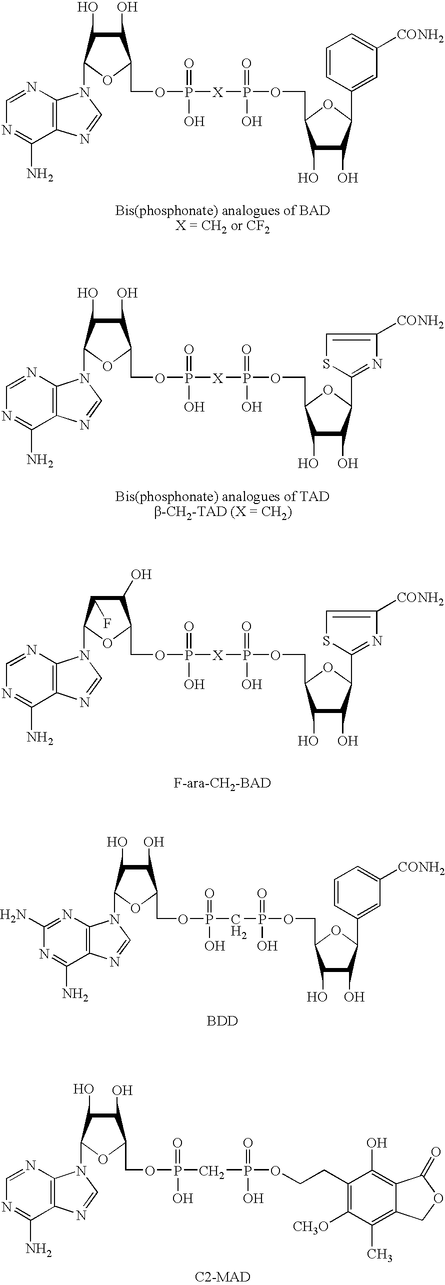 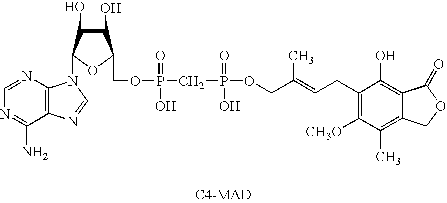
or its pharmaceutically acceptable salt thereof, wherein X is oxygen, sulfur, methylene, monofluoromethylene or difluoromethylene.

38. A method for the treatment or prophylaxis of abnormal cellular proliferation in a host, comprising administering an effective amount of a compound of the formula: 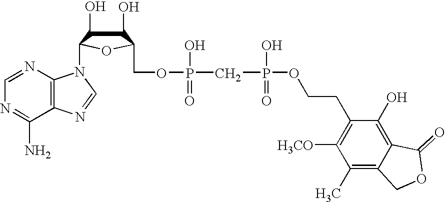
or its pharmaceutically acceptable salt thereof, optionally in a pharmaceutically acceptable carrier or diluent.

39. A method for the treatment or prophylaxis of abnormal cellular proliferation in a host, comprising administering an effective amount of a compound selected from the group consisting of the following: 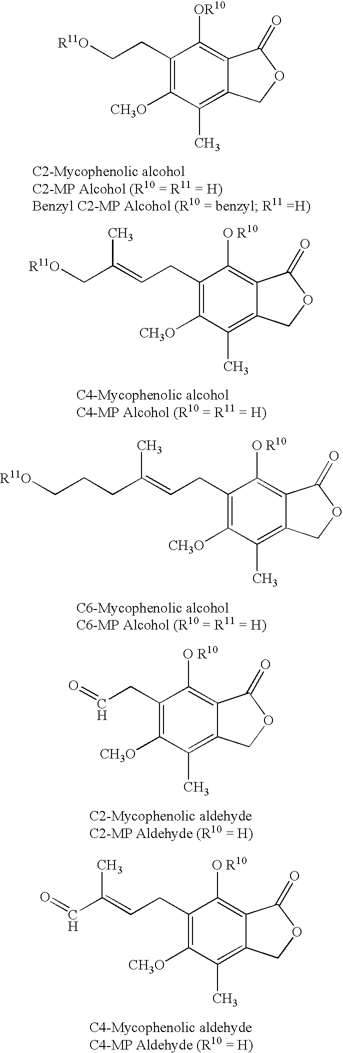 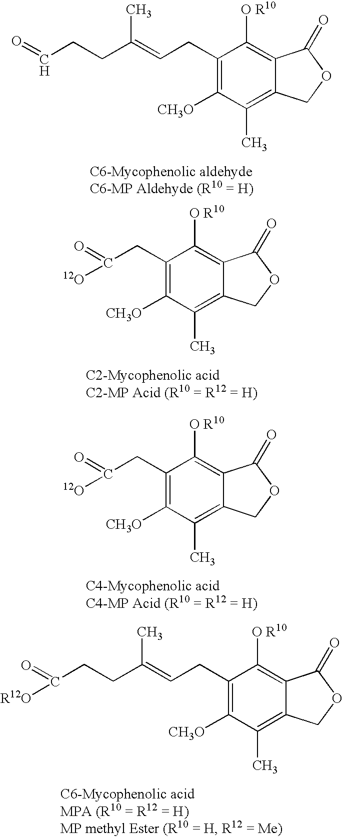
or its pharmaceutically acceptable salt thereof; wherein
each R¹⁰ and R¹¹ is independently hydrogen, alkyl, acyl, benzyl or methoxymethyl (MOM) group, and each R¹² is independently hydrogen, alkyl or aryl;
optionally in a pharmaceutically acceptable carrier or diluent.

40. A method for the treatment or prophylaxis of abnormal cellular proliferation in a host, comprising administering an effective amount of a compound selected from the group consisting of the following: 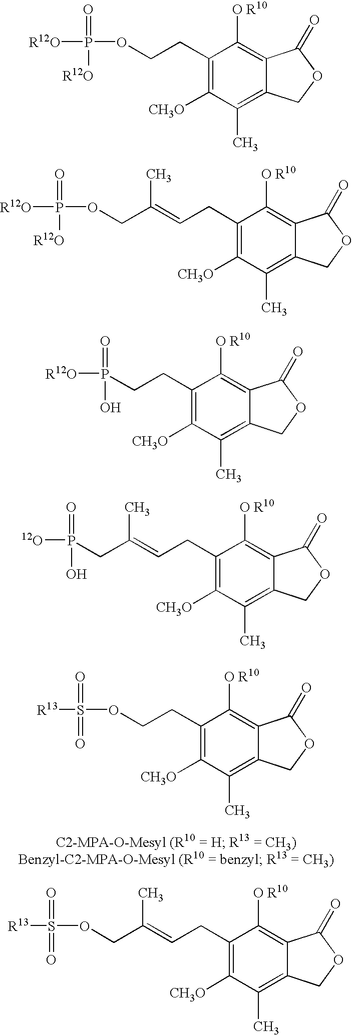
or its pharmaceutically acceptable salt thereof; wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
optionally in a pharmaceutically acceptable carrier or diluent.

41. A method for the treatment or prophylaxis of abnormal cellular proliferation in a host, comprising administering an effective amount of a compound selected from the group consisting of the following: 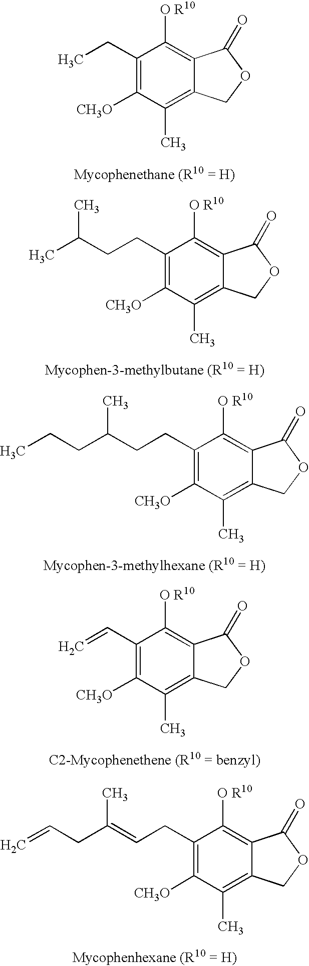
or its pharmaceutically acceptable salt thereof; wherein
each R¹⁰ is independently hydrogen, alkyl, acyl, benzyl or methoxymethyl (MOM) group;
optionally in a pharmaceutically acceptable carrier or diluent.

42. A method for the treatment or prophylaxis of abnormal cellular proliferation in a host, comprising administering an effective amount of a compound selected from the group consisting of the following: 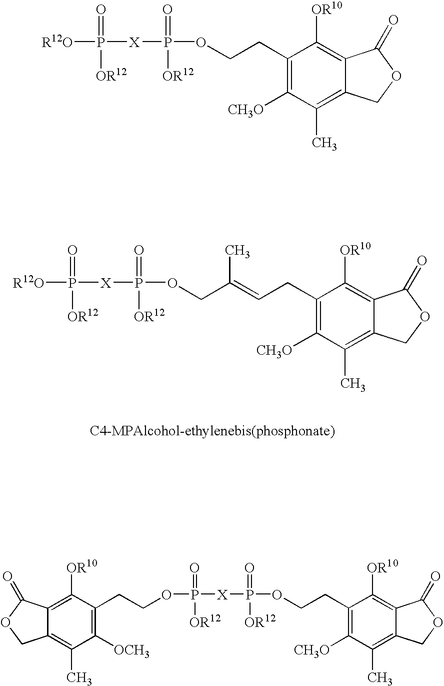
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
optionally in a pharmaceutically acceptable carrier or diluent.

43. A method for the treatment or prophylaxis of abnormal cellular proliferation in a host, comprising administering an effective amount of a compound of the formula (I): 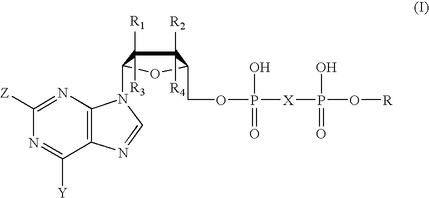
or its pharmaceutically acceptable salt thereof; wherein
R is 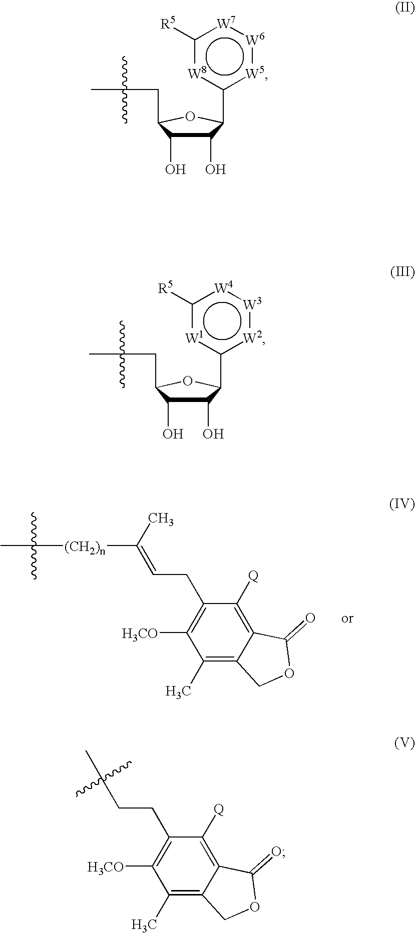
X is oxygen, sulfur, methylene, monofluoromethylene or difluoromethylene;
Y is hydrogen, halogen (F, Cl, Br, I), NH₂, NHR⁶, NR⁶R⁷, NHOH, NHOR⁶, NHNH₂, NR⁶NH₂, NHNHR⁶, SH, SR⁶, OH or OR⁶;
Z is hydrogen, halogen (F, Cl, Br, I), NH₂, NHR⁸, NR⁸R⁹, NHOH, NHOR⁸, NHNH₂, NR⁸NH₂, NHNHR⁸, SH, SR⁸, OH, OR⁸;
W¹-W⁴ are same or different, and independently methyne (—CH═), azomethyne (—N═) or sulfur;
W⁵-W⁸ are same or different, and independently methyne (—CH═) or azomethyne (—N═);
R¹, R², R³ and R⁴ are independently hydrogen, hydroxyl or fluorine;
R⁵ is halogen (F, Cl, Br, I), CN, CONH₂, CO₂Me, CO₂Et or CO₂H; and
R⁶, R⁷, R⁸ and R⁹ are independently a lower alkane or alkene of 1, 2, 3, 4, 5 or 6 carbons or aryl or aralkyl;
wherein the compound is specifically not tiazole-4-carboxamide adenine dinucleotide (TAD) or benzamide adenine dinucleotide (BAD);
in combination or alternation with one or more other effective antiproliferative agent, optionally in a pharmaceutically acceptable carrier or diluent.

44. The method of claim 29, wherein the compound of formula (I) is selected from the group consisting of the following: 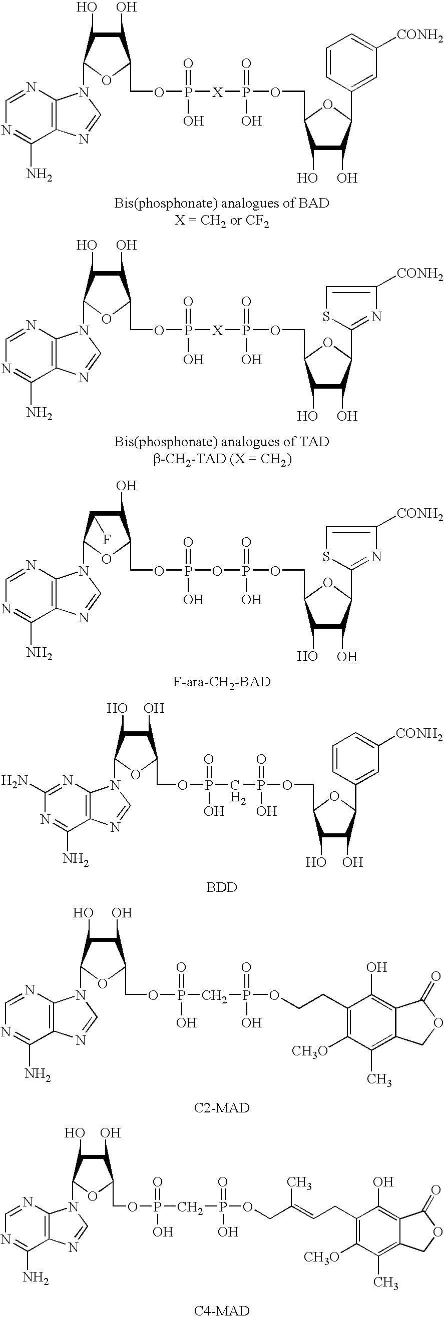
or its pharmaceutically acceptable salt thereof, wherein X is oxygen, sulfur, methylene, monofluoromethylene or difluoromethylene.

45. A method for the treatment or prophylaxis of abnormal cellular proliferation in a host, comprising administering an effective amount of a compound of the formula; 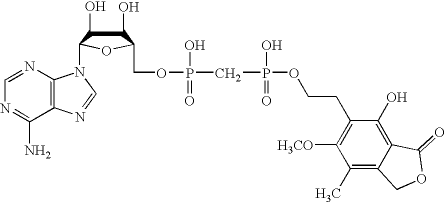
or its pharmaceutically acceptable salt thereof, in combination or alternation with one or more other effective antiproliferative agent, optionally in a pharmaceutically acceptable carrier or diluent.

46. A method for the treatment or prophylaxis of abnormal cellular proliferation in a host, comprising administering an effective amount of a compound selected from the group consisting of the following: 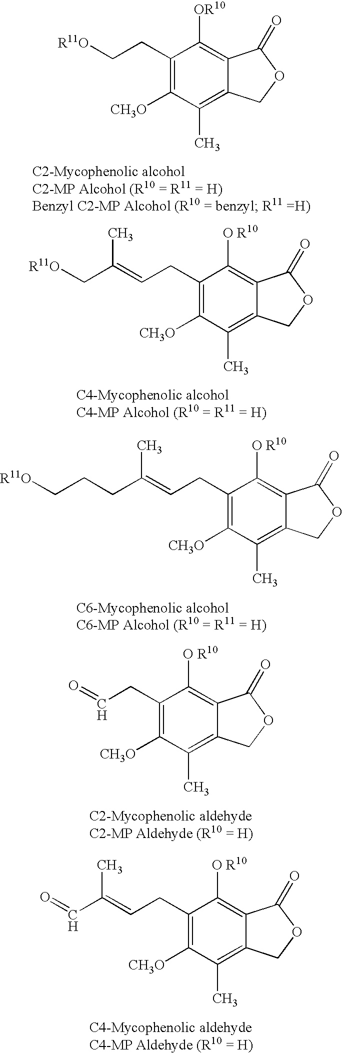 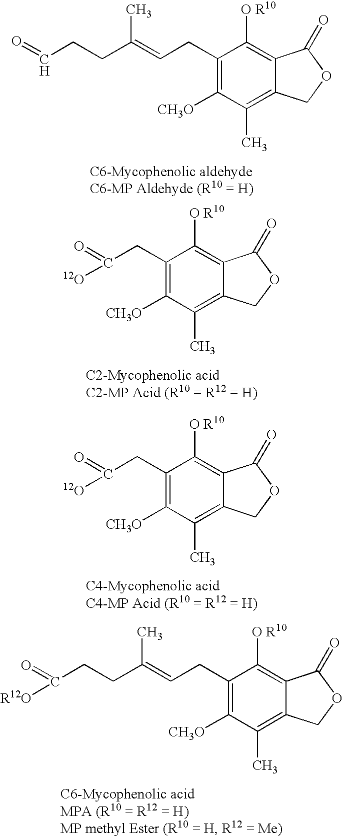
or its pharmaceutically acceptable salt thereof; wherein
each R¹⁰ and R¹¹ is independently hydrogen, alkyl, acyl, benzyl or methoxymethyl (MOM) group, and each R¹² is independently hydrogen, alkyl or aryl;
in combination or alternation with one or more other effective antiproliferative agent, optionally in a pharmaceutically acceptable carrier or diluent.

47. A method for the treatment or prophylaxis of abnormal cellular proliferation in a host, comprising administering an effective amount of a compound selected from the group consisting of the following: 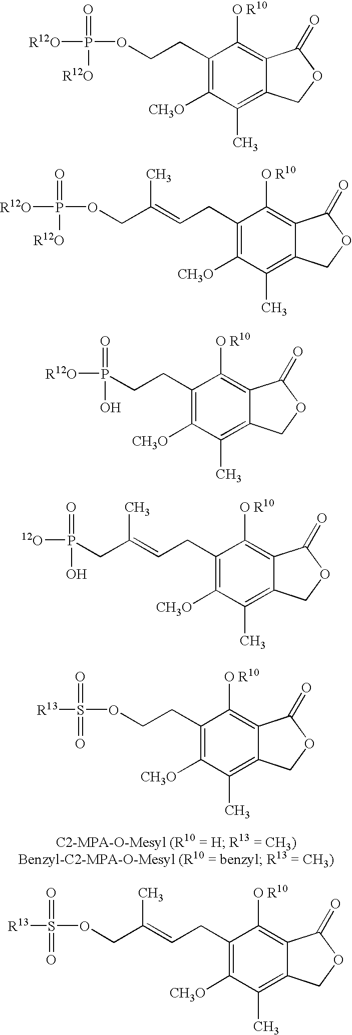
or its pharmaceutically acceptable salt thereof; wherein
each R¹⁰ is independently hydrogen, alkyl, acyl, benzyl or methoxymethyl (MOM) group;
each R¹² is independently hydrogen, alkyl or aryl; and
R¹³ is lower alkyl (i.e. a C₁, C₂, C₃, C₄, C₅ or C₆ alkyl), lower alkenyl (i.e. a C₂, C₃, C₄, C₅ or C₆ alkenyl), lower alkynyl (i.e. a C₂, C₃, C₄, C₅ or C₆ alkynyl) or a C₃-C₈ cycloalkyl;
in combination or alternation with one or more other effective antiproliferative agent, optionally in a pharmaceutically acceptable carrier or diluent.

48. A method for the treatment or prophylaxis of abnormal cellular proliferation in a host, comprising administering an effective amount of a compound selected from the group consisting of the following: 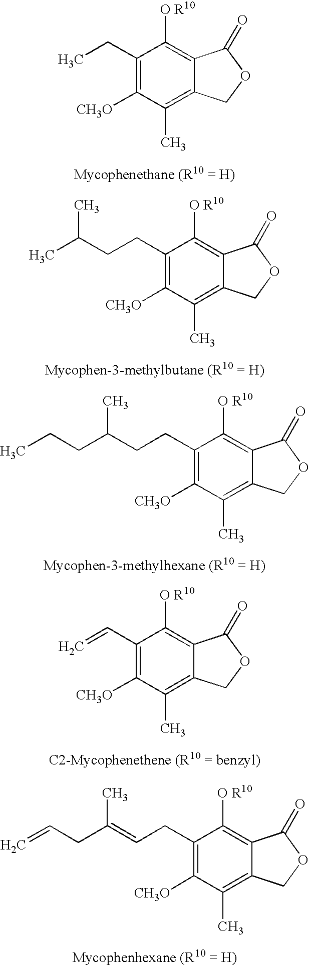
or its pharmaceutically acceptable salt thereof; wherein
each R¹⁰ is independently hydrogen, alkyl, acyl, benzyl or methoxymethyl (MOM) group;
in combination or alternation with one or more other effective antiproliferative agent, optionally in a pharmaceutically acceptable carrier or diluent.

49. A method for the treatment or prophylaxis of abnormal cellular proliferation in a host, comprising administering an effective amount of a compound selected from the group consisting of the following: 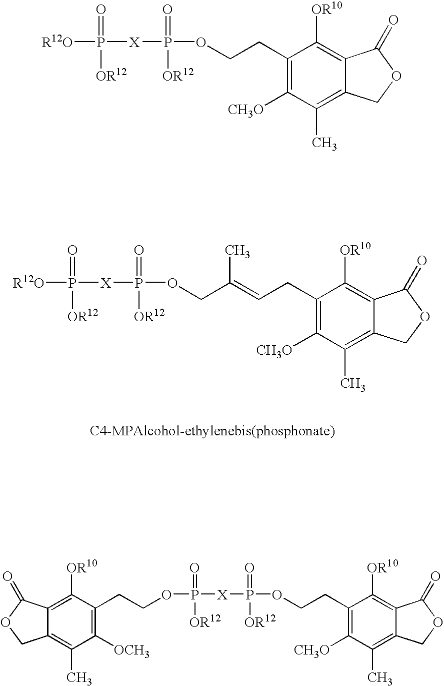
or its pharmaceutically acceptable salt thereof, wherein
X is oxygen, sulfur, methylene, monofluoromethylene or difluoromethylene; and
each R¹⁰ is independently hydrogen, alkyl, acyl, benzyl or methoxymethyl (MOM) group; and
each R¹² is independently hydrogen, alkyl or aryl;
in combination or alternation with one or more other effective antiproliferative agent, optionally in a pharmaceutically acceptable carrier or diluent.

50. The method of any one of claims 22-49, wherein the host is a human.
